(19) United States
(12) Patent Application Publication
(10) Pub. No.: US 2010/0196355 A1
(43) Pub. Date: Aug. 5, 2010

(54) Immunophilin Ligands and Methods for Modulating Immunophilin and Calcium Channel Activity

(75) Inventors: Edmund Idris Graziani, Chestnut Ridge, NY (US); Benfang Helen Ruan, Acton, MA (US); Kevin Pong, Robbinsville, NJ (US); Mark Robert Bowlby, Richboro, PA (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/524,093

(22) PCT Filed: Jan. 29, 2007
(86) PCT No.: PCT/US07/02656

§ 371 Date: Feb. 12, 2010

(51) Int. Cl. A61K 39/395; C07K 14/47; C07K 16/40; C07D 498/22; A61K 31/5365; A61P 25/28; A61P 25/06; A61P 25/18; A61P 25/22; A61P 25/24; A61P 25/14; A61P 25/16; A61P 25/08; A61P 13/06; A61P 9/06; A61P 9/12; A61P 9/10; C12N 11/00; C12N 5/00

(52) U.S. Cl. 424/130.1; 530/350; 530/389.1; 544/63; 514/229.5; 435/183; 435/375

(57) ABSTRACT

Immunophilin ligands and their uses as modulators of calcium channel activity are disclosed. Screening, therapeutic and prophylactic methods for conditions associated with calcium channel dysfunction, e.g., neurodegenerative and cardiovascular disorders, are also disclosed.

### BACKGROUND

Entry of calcium into mammalian cells through voltage-gated calcium channels mediates a wide variety of cellular and physiological responses, including excitation-contraction coupling, hormone secretion and gene expression (Miller et al. (1987) Science 235:46-52; Augustine et al. (1987) Annu. Rev. Neurosci. 10:633-93). Calcium channels directly affect membrane potential and contribute to diverse electrical properties in neurons. Calcium entry further influences neuronal function by regulating calcium-dependent ion channels and modulating the activity of calcium-dependent enzymes, such as protein kinase C and calmodulin-dependent protein kinase II. An increase in calcium concentration at the presynaptic nerve terminal typically triggers neurotransmitter release and increases in calcium channel activity. Such calcium increases have been implicated in a number of human disorders, including, but are not limited to, neurological and cardiac disorders (e.g., congenital migraine, cerebellar ataxia, angina, epilepsy, hypertension, ischemia, and some arrhythmias).

In view of the widespread role of voltage-gated calcium channels in physiological and pathological functions, the need still exists for identifying novel modulators of calcium channel activity and understanding their mechanism of action.

### SUMMARY

Methods and compositions for modulating immunophilin and calcium channel activity are disclosed. In one embodiment, immunophilin ligands modified at the mTOR binding region of rapamycin have been shown to decrease the activity of FKBP52 and voltage gated L-type calcium channels, in particular, the β1 subunits of the L-type calcium channels. Such decreased activity has been shown to be associated with a concomitant increase in neurite outgrowth and neuronal survival. Without being bound by theory, it is believed that the decrease in FKBP52 and channel activity occurs, at least in part, via the formation of a complex that includes an immunophilin ligand, one or both of an immunophilin (e.g., FKBP52) and/or the β1 subunit of the L-type calcium channel. Accordingly, the present invention provides methods for modulating, e.g., inhibiting, decreasing and/or reducing, the activity of the immunophilin and/or the β subunit of the L-type calcium channel using immunophilin ligands, e.g., immunophilin ligands modified at the mTOR binding region. In other aspects, methods for treating or preventing conditions associated calcium channel dysfunction, e.g., neurodegenerative and cardiovascular disorders, using immunophilin ligands are also disclosed. Methods and reagents of identifying compounds that modulate an activity of the immunophilin and/or the calcium channel subunit are additionally encompassed by the invention.

In one aspect, the invention provides a purified complex that includes an immunophilin ligand (e.g., a rapamycin or a meridamycin analogue (e.g., a known or an unknown analogue)), and one or both of (i) an immunophilin or a functional variant thereof, and/or (ii) a calcium channel subunit or a functional variant thereof. Accordingly, exemplary complexes of the invention may include an immunophilin ligand and an immunophilin or functional fragment thereof; an immunophilin ligand and a calcium channel subunit or a functional variant thereof; and an immunophilin ligand, an immunophilin or a functional variant thereof, and a calcium channel subunit or a functional variant thereof. It shall be understood that the complexes of the invention may include additional polypeptides or fragments thereof.

In one embodiment, the rapamycin analogue is modified at the mTOR binding region of rapamycin, e.g., has a heteroatom substituent at positions 1 and 4 of the rapamycin backbone (see FIG. 1A). In other embodiments, the rapamycin analogue has a cyclic structure at positions 1, 2, 3 and/or 4 of the rapamycin backbone. In other embodiments, the rapamycin analogue has a chemical formula as described herein (e.g., formulae I, Ia and/or Ib). In other embodiments, the rapamycin analogue has the structure of the compounds referred to herein as “rapamycin I” and “rapamycin II” (FIG. 1A) (also referred to herein as “Compound I” and “Compound II,” respectively). In other embodiments, the immunophilin ligand binds to an immunophilin, e.g., FKBP-52, with a selectivity, relative to other immunophilins (e.g., FKBP12), that is at least 100, 200, 300, 400, 500, 600, 700, 800 or higher than that of rapamycin.

In embodiments, the immunophilin is an FK506 binding protein, e.g., FKBP52 (e.g., a mammalian FKBP52), or a functional variant thereof. In other embodiments, the calcium channel subunit is a subunit of the voltage gated L-type calcium channel, e.g., a β1 subunit (e.g., a mammalian β1 subunit), or a functional variant thereof. A functional variant of a polypeptide described herein includes a fragment, mutated form, fusion protein, labeled form (e.g., radiolabeled) that retains one or more activities of the unmodified form, e.g., retains the ability to bind to an immunophilin ligand and/or form a complex as described herein. The terms “immunophilin” and “calcium channel,” or the like, include “functional variants thereof,” although the phrase “functional variants thereof” may or may not be repeated throughout for ease of reading.

In another aspect, the invention provides a method, or an assay, for identifying a test compound (e.g., a rapamycin or a meridamycin analogue as described herein) that interacts with (e.g., binds to) and/or modulates (e.g., decreases or increases) an activity of (i) an immunophilin, e.g., an immunophilin as described herein (e.g., FKBP52), (or a functional variant thereof), and/or (ii) a calcium channel subunit (e.g., a calcium channel subunit as described herein (e.g., β1 subunit)), (or a functional variant thereof). The method, or the assay, includes: contacting the immunophilin, and/or the calcium channel subunit, with a test compound under conditions that allow an interaction and/or modulation of activity to occur; detecting a change in the interaction and/or activity of the immunophilin and/or the calcium channel subunit in the presence of the test compound relative to a reference, e.g., a reference sample (e.g., a control sample not exposed to the test compound, or a control sample exposed to rapamycin). A change (e.g., an increase or a decrease) in the level of interaction and/or activity of the immunophilin and/or the calcium channel subunit, in the presence of the test compound, relative to the reference, indicates that said test compound interacts with and/or affects (e.g., increases or decreases) the activity of the immunophilin and/or a calcium channel subunit.

In embodiments, the interaction between the test compound and one or both of the immunophilin and/or the calcium channel subunit is detected by the formation of a complex (e.g., a complex between one or more of the following: the test compound and the immunophilin; the test compound and the calcium channel subunit; or, the test compound, the immunophilin and the calcium channel subunit). A change in the formation and/or stability of the complex in the presence of the test compound, relative to the reference indicates that said test compound interacts with one or both of the immunophilin and/or a calcium channel subunit.

In yet another aspect, the invention provides a method, or an assay, for identifying a neurotrophic and/or neuroprotective compound. The method, or the assay, includes: contacting (i) an immunophilin (e.g., an immunophilin as described herein (e.g., FKBP52)) (or a functional variant thereof), and/or a (ii) calcium channel subunit (e.g., a calcium channel subunit as described herein (e.g., β1 subunit)) (or a functional variant thereof), with a test compound under conditions that allow the interaction and/or modulation of activity to occur; detecting a change in the interaction and/or activity of the immunophilin and/or the calcium channel subunit in the presence of the test compound relative to a reference, e.g., a reference sample (e.g., a control sample not exposed to the test compound, or a control sample exposed to rapamycin). An increase in the level of interaction, and/or a decrease in the activity of the immunophilin and/or the calcium channel subunit, in the presence of the test compound, relative to the reference, is indicative of a potential neurotrophic and/or neuroprotective compound. In embodiments, the increase in the interaction between the test compound and the immunophilin and/or the calcium channel subunit is detected by an increase in the formation and/or stability of a complex between two or more of the aforesaid components. In other embodiments, the decrease in activity is determined by detecting a decrease in calcium channel activity, e.g., as described in more detail herein. A decrease in immunophilin activity can be detected by, e.g., measuring glucocorticoid receptor activation.

Additional embodiments of the aforesaid screening methods and assays may include one or more of the following features:

In embodiments, the immunophilin and/or the calcium channel subunit are present in a sample. The sample can be a cell lysate or a reconstituted system (e.g., cell membrane or soluble components). Alternatively, the sample can include cells in culture, e.g., purified cultured or recombinant cells, or in vivo in an animal subject. A change in the interaction and/or activity between the test compound or neurotrophic compound and the immunophilin and/or the calcium channel subunit can be determined by detecting one or more of: a change in the binding or physical formation of the complex itself, e.g., by biochemical detection, affinity based detection (e.g., Western blot, affinity columns), immunoprecipitation, fluorescence resonance energy transfer (FRET)-based assays, spectrophotometric means (e.g., circular dichroism, absorbance, and other measurements of solution properties); a change, e.g., an increase or a decrease, in signal transduction, e.g., calcium-dependent phosphorylation and/or transcriptional activity (e.g., a transcriptional profile as described herein); a change, e.g., increase or decrease, in calcium channel activity (e.g., electrophysiological activity, calcium kinetics), and/or a change, e.g., increase or decrease, in neuronal survival, differentiation and/or neurite outgrowth. In one embodiment, the test compound or the neurotrophic compound is identified and re-tested in the same or a different assay. For example, a test compound or a neurotrophic compound is identified in an in vitro or cell-free system, and re-tested in an animal model or a cell-based assay. Any order or combination of assays can be used. For example, a high throughput assay can be used in combination with an animal model or tissue culture.

In other embodiments, the method, or assay, includes providing a step based on proximity-dependent signal generation, e.g., a two-hybrid assay that includes a first fusion protein (e.g., a fusion protein comprising an immunophilin portion), and a second fusion protein (e.g., a fusion protein comprising a β subunit portion), contacting the two-hybrid assay with a test compound, under conditions wherein said two hybrid assay detects a change in the formation and/or stability of the complex, e.g., the formation of the complex initiates transcription activation of a reporter gene.

In other embodiments, the method, or assay, further includes the step of contacting the immunophilin and/or the calcium channel subunit with a known immunophilin ligand (e.g., a rapamycin analogue modified at the mTOR binding region of rapamycin as described herein); detecting the interaction and/or activity of the known immunophilin ligand with the immunophilin and/or the calcium channel subunit in the absence or presence of a test compound. A change in binding (e.g., complex formation) and/or activity of the immunophilin and/or the calcium channel subunit, in the presence or absence of the test compound, is indicative that the test compound interacts with and/or binds to the immunophilin and/or the calcium channel subunit.

In other embodiments, the method, or assay, further includes the step(s) of comparing binding of the test compound to the complex compared to the binding of the known immunophilin ligand to the complex. The method, or assay, can additionally, optionally, include detecting the interaction (e.g., binding) of the test compound to a complex of the immunophilin and/or the calcium channel subunit, relative to the individual components.

In some embodiments, the method further includes the step of evaluating a change, e.g., increase or decrease, in neuronal activity, e.g., one or more of neuronal survival, differentiation and/or neurite outgrowth. An increase in one or more of neuronal survival, differentiation and/or neurite outgrowth is indicative of a neurotrophic and/or neuroprotective compound. The evaluation step can be performed in cells in culture or in an animal model as described herein.

Candidate test or neurotrophic compounds increase the formation of the complex described herein and/or inhibit calcium channel or immunophilin activity. In one embodiment, the test compound binds with higher affinity to the complex relative to its binding to the individual components of the complex. The test or neurotrophic compound can be a natural product or a chemically synthesized compound. For example, the test compound can be a polyketide obtained from a naturally-occurring or modified (e.g., recombinantly modified) prokaryotic (e.g., Actinomycete such as Streptomyces, e.g. S. hygroscopicus) or eukaryotic (e.g., a fungal or mammalian) cell. In embodiments, the test compound is a rapamycin or a meridamycin, or an analogue thereof (e.g., a rapamycin or meridamycin compound described herein, or an analogue thereof).

Compounds disclosed herein and/or identified by the methods or assays described herein are also within the scope of the invention. Compositions, e.g., pharmaceutical compositions, that include the compounds of the invention and a pharmaceutically-acceptable carrier are disclosed. In one embodiment, the compositions include the compounds of the invention in combination with one or more agents, e.g., therapeutic agents. In one embodiment, the second agent is a calcium channel antagonist, e.g., an antagonist of an L-type calcium channel. Examples of antagonists of L-type calcium channels include dihydropyridines, phenylalkylamines and benzothiazepines diphenylbutylpiperidine class of antischizophrenic neuroleptic drugs. In certain embodiments, the amount of the immunophilin ligand and/or calcium channel antagonist administered present in the composition is lower than the amount of the drug present in compositions administered individually.

In another aspect, the invention provides a host cell comprising one or more nucleic acids encoding one or more of the polypeptide constituents of the complex disclosed herein. In one embodiment, the host cell contains a first nucleic acid that includes a nucleotide sequence encoding an immunophilin, e.g., an FKBP52 (e.g., a mammalian FKBP52) (or a functional variant thereof); and/or a second nucleic acid that includes a nucleotide sequence encoding a subunit of the voltage gated L-type calcium channel, e.g., a β1 subunit (e.g., a mammalian (β1 subunit), (or a functional variant thereof). In some embodiments, recombinant immunophilin and the calcium channel subunit and/or control regulatory sequences thereof are exogenously added.

In yet another aspect, the invention provides an antibody, or antigen-binding fragment thereof, that binds to the complexes disclosed herein. In certain embodiments, the antibody or fragment thereof increases the formation of a complex disclosed herein.

In other embodiments, the antibody or fragment thereof decreases or inhibits the formation of a complex disclosed herein. In one embodiment, the antibody or fragment thereof selectively binds to the complex, but does not significantly bind to the individual components of the complex. The complex can include the immunophilin ligand or test compound and the immunophilin and/or the calcium channel, as described herein.

In another aspect, the invention provides a method of making an antibody or antigen binding fragment thereof. The method includes using the complex described herein as an antigen (e.g., an immunogen in an animal model or phage display selection), and selecting antibodies or binding fragments thereof on the basis of binding to the complex. The method may, optionally, include the step of confirming binding of the antibody or fragment thereof to the complex and comparing binding of the antibody to the individual components of the complex, or a complex that contains the three components of the complex. Antibodies or fragments thereof that selectively bind to the complex over the individual components or a complex thereof are preferred.

In another aspect, the invention provides a method of modulating (e.g., decreasing) the activity of an immunophilin (or a functional variant thereof), and/or a calcium channel subunit (or a functional variant thereof). The method includes: contacting one or both of (i) an immunophilin, e.g., an FKBP52, as described herein; and/or (ii) a subunit of a calcium channel, e.g., a β1 subunit, as described herein, with an immunophilin ligand (e.g., a rapamycin or meridamycin analogue as described herein), under conditions that allow an interaction (e.g., binding) to occur. In embodiments, the activity modulated (e.g., increased) is the formation and/or stability of a complex that includes the immunophilin ligand, and one or both of the immunophilin, and/or the calcium channel subunit. In one embodiment, the contacting step can be effected in vitro, e.g., in a cell lysate or in a reconstituted system. Alternatively, the subject method can be performed on cells in culture, e.g., in vitro or ex vivo. For example, cells (e.g., purified or recombinant cells) can be cultured in vitro and the contacting step can be effected by adding the immunophilin ligand, e.g., the rapamycin or meridamycin analogue, to the culture medium. Typically, the cell is a mammalian cell, e.g., a human cell. In some embodiments, the cell is a neuronal or a cardiovascular cell.

In another aspect, the invention provides a method of modulating, e.g., inhibiting, calcium channel activity (e.g., voltage-gated calcium channel activity) and/or immunophilin activity, in a cell. The method includes: contacting a cell that expresses (i) an immunophilin, e.g., an FKBP52 (e.g., a mammalian FKBP52) (or a functional variant thereof); and/or (ii) a subunit of the voltage gated L-type calcium channel, e.g., a β1 subunit (e.g., a mammalian (β1 subunit), (or a functional variant thereof), with an immunophilin ligand, e.g., a rapamycin or meridamycin analogue as described herein, under conditions that allow an interaction between (e.g., formation of a complex that includes) the ligand, and one or both of the immunophilin and/or the subunit to occur, thereby inhibiting the calcium channel and/or immunophilin activity. Typically, the cell is a mammalian cell, e.g., a human cell. In some embodiments, the cell is a neuronal or a cardiovascular cell. The method can be performed in cells in cultured medium as described herein.

In yet another aspect, the invention provides a method of increasing neuronal function, e.g., neurite outgrowth and/or survival. The method includes: contacting a neuronal cell with an immunophilin ligand in an amount sufficient to promote neuronal function. In embodiments, the immunophilin ligand is present at a concentration that elicits one or more of the following: (i) downregulates expression and/or activity at least one component of the calcium signaling pathways (e.g., calcium- influx channels, N-methyl D-aspartate subtype of glutamate (NMDA) receptors, plasminogen activator (PLAU), SHT3R channels); (ii) decreases immunophilin (e.g., FKBP52) activity and/or expression; (iii) reduces or inhibits the activity and/or expression of a calcium channel (e.g., an L-type calcium channel); (iv) activates steroid receptor signaling (e.g., glucocorticoid receptor signaling); (v) induces formation of a complex that includes the immunophilin ligand, the immunophilin (e.g., FKBP52) and/or a subunit of the voltage gated L-type calcium channel, e.g., a β1 subunit; and/or (vi) protects neurons from calcium-induced cell death.

In yet another aspect, the invention features a method of treating or preventing, in a subject, a disorder associated with calcium channel dysfunction(e.g., a disorder associated with L-type calcium channel function). In embodiments, the disorder is not associated with a ryanodine receptor channelopathy. The method includes administering to a subject an immunophilin ligand in an amount sufficient to treat or prevent the disorder. In embodiments, the immunophilin ligand is present at a concentration that elicits one or more of the following: (i) downregulates expression or activity at least one component of the calcium signaling pathways (e.g., calcium- influx channels, NMDA receptors, plasminogen activator (PLAU), SHT3R channels); (ii) decreases immunophilin (e.g., FKBP52) activity and/or expression; (iii) reduces or inhibits the activity and/or expression of a calcium channel (e.g., an L-type calcium channel); (iv) activates steroid receptor signaling (e.g., glucocorticoid receptor signaling); (v) induces formation of a complex that includes the immunophilin ligand, the immunophilin (e.g., FKBP52) and/or a subunit of the voltage gated L-type calcium channel, e.g., a β1 subunit; and/or (vi) protects neurons from calcium-induced cell death.

Additional embodiments of the aforesaid methods of modulating activity and treating or preventing disorders may include one or more of the following features.

In one embodiment, the immunophilin ligand is a rapamycin analogue which is modified at the mTOR binding region, e.g., has a heteroatom substituent at positions 1 and 4 of the rapamycin backbone (see FIG. 1A). In other embodiments, the rapamycin analogue has a cyclic structure at positions 1, 2, 3 and/or 4 of the rapamycin backbone. In other embodiments, the rapamycin analogue has a chemical formula as described herein (e.g., formulae I, Ia and/or Ib). In other embodiments, the rapamycin analogue has the structure of the compounds referred to herein as “rapamycin I” and “rapamycin II” (FIG. 1A). In other embodiments, the immunophilin ligands binds to an immunophilin, e.g., FKBP-52, with a selectivity, relative to another immunophilin (e.g., FKBP-12), that is at least 100, 200, 300, 400, 500, 600, 700, 800 or higher than that of rapamycin.

In other embodiments, the method can be performed on cells (e.g., neuronal cells) present in a subject, e.g., as part of an in vivo (e.g., therapeutic or prophylactic) protocol, or in an animal subject (e.g., an in vivo animal model). For in vivo methods, the immunophilin ligand, e.g., the rapamycin or meridamycin analogue, alone or in combination with another agent, can be administered to a subject, e.g., a mammal, suffering from a disorder, e.g., a neurodegenerative or a cardiovascular disorder, in an amount sufficient to form and/or stabilize the complex.

In some embodiments, a therapeutic amount or dosage can be determined, e.g., prior to administration to the subject, by testing in vitro the amount of immunophilin ligand required to elicit one or more of the following: (i) induce complex formation; (ii) downregulate expression or activity at least one component of the calcium signaling pathways; (iii) reduce or inhibit the activity of a calcium channel (e.g., an L-type calcium channel); and/or (iv) activate steroid receptor signaling (e.g., glucocorticoid receptor signaling). The in vivo method can, optionally, include the step(s) of identifying (e.g., evaluating, diagnosing, screening, and/or selecting) a subject at risk of having, or having, one or more symptoms associated with a disorder associated with calcium channel dysfunction (e.g., a disorder associated with L-type calcium channel function). In embodiments, the disorder is not associated with a ryanodine receptor channelopathy.

The subject can be a mammal, e.g., a human, suffering from, for example, a neurodegenerative or a cardiovascular disorder. In embodiments, the subject is a mammal having one or more symptoms associated with a disorder associated with calcium channel dysfunction (e.g., a disorder associated with L-type calcium channel function). In embodiments, the disorder is not associated with a ryanodine receptor channelopathy. For example, the subject is a mammal (e.g., a human patient) suffering from a disorder chosen from one or more of: stroke, Parkinson's disease, epilepsy, angina, cardiac arrhythmia and ischemia. In other embodiments, the subject is a mammal suffering from one or more of: migraine, neuropathic pain, acute pain, mood disorders, schizophrenia, depression, anxiety, cerebellar ataxia, tardive dyskinesia, hypertension and/or urinary incontinence.

The immunophilin ligand, e.g., the rapamycin or meridamycin analogue, can be administered to the subject alone, or in combination with one or more agents, e.g., therapeutic agents. In one embodiment, the second agent is a calcium channel antagonist, e.g., an antagonist of an L-type calcium channel. Examples of antagonists of L-type calcium channels include dihydropyridines, phenylalkylamines and benzothiazepines diphenylbutylpiperidine class of antischizophrenic neuroleptic drugs. In certain embodiments, the amount of the immunophilin ligand and/or calcium channel antagonist administered in combination is lower than the amount of the drug administered individually. The agents can be administered simultaneously or sequentially.

In yet another aspect, the invention provides a method of stimulating one or more of neurite outgrowth, survival, and/or differentiation of a neuronal cell (e.g., a dopaminergic, cholinergic, cortical, and spinal cord neuronal cell). The method includes contacting the cell with an antagonist of an immunophilin (e.g., FKBP52) and/or a calcium channel β subunit, e.g., a β1 subunit of the voltage gated L-type calcium channel. The antagonist can also be an inhibitor of activity and/or expression of the immunophilin (e.g., FKBP52) or calcium channel β subunit. In one embodiment, the inhibitor is an intracellular antagonist of a calcium channel, e.g., an antagonist of a calcium channel β subunit. In another embodiment, the antagonist is an immunophilin ligand, e.g., a rapamycin or meridamycin analogue as described herein. Typically, the immunophilin ligand is administered in an amount sufficient to form and/or stabilize a complex that includes the ligand, an immunophilin (or a functional variant thereof), and/or a calcium channel subunit (or a functional variant thereof). In other embodiment, the antagonist is an inhibitor of transcription of the immunophilin (e.g., FKBP52) and/or calcium channel β subunit, e.g., RNAi. The contacting step can be effected in vitro, e.g., in culture, or in vivo, e.g., by administration to a subject, as described herein.

As used herein, the articles “a” and “an” refer to one or to more than one (e.g., to at least one) of the grammatical object of the article.

The term “or” is used herein to mean, and is used interchangeably with, the term “and/or”, unless context clearly indicates otherwise.

The terms “proteins” and “polypeptides” are used interchangeably herein.

Other features, objects, and advantages of the invention will be apparent from the description and drawings, and from the claims.

### DESCRIPTION OF DRAWINGS

FIG. 1A provides a diagram of chemical synthesis and structures of rapamycin analogues I and II (referred to interchangeably in the Figure (and throughout) as “1” and “2,” or “Compound 1” and “Compound 2,” respectively). The rapamycin structure using the numbering system referenced herein is also provided.

FIG. 1B provides a bar graph depicting promotion of neuronal survival in cortical neurons in response to rapamycin analogue I (referred to in the Figure as “Compound 1”).

FIG. 1C provides a graph depicting neurite outgrowth in cortical neurons in response to rapamycin analogue I (referred to in the Figure as “Compound 1”).

FIG. 1D provides a graph depicting neurite outgrowth in F-11 cells in response to rapamycin analogue I (referred to in the Figure as “Compound 1”).

FIG. 2 provides a diagram showing preparation of affinity matrices of several rapamycin analogues I, II, FK506 and rapamycin.

FIG. 3 provides an SDS-PAGE gel photograph of the mobility of the proteins isolated by affinity precipitation from lysates of F11 cells (fusion between mouse embryonic neuroblastoma and rat dorsal root ganglion (DRG) neurons).

FIG. 4 provides Fourier transform ion cyclotron resonance mass spectrometric (FT-ICR-MS) analysis of tryptic digested bands from the SDS-PAGE gel. “Rap. An. I” represents rapamycin analogue I.

FIGS. 5A-5D depict the characterization of immunophilin binding of rapamycin analogues I and II.

FIG. 5A provides an SDS-PAGE gel analysis of proteins that bound to the various affinity matrixes. The bands found in the marker lane are (1) 220 kDa, (2) 78 kDa, (3) 45.7 kDa, in the rapamycin analogue I pull-down fraction are (4) Myosin, (5) FKBP52, (6) CACNB 1, FKBP25 and FKBP 12, in the blank bead control is (7) actin, and in the rapamycin analogue II pull-down fraction are (5) FKBP52, and (6) CACNB 1. “Compound 1” represents rapamycin analogue I and “Compound II” represents rapamycin analogue II.

FIG. 5B provides a Western blot analysis using anti-FKBP52 and anti-Ca²⁺ channel β₁-subunit antibodies to detect the presence of the corresponding antigens on affinity beads coating with rapamycin analogue I and FK506.

FIG. 5C are bar graphs depicting the results of size exclusion chromatography to measure the fraction of [¹⁴C]-1 that binds to the purified recombinant immunophilins and cyclophilins.

FIG. 5D is a blot depicting the results of affinity chromatography to test the binding of FKBP25 and PPID proteins to Compound 2. Lanes were labeled as follows: “C” represent a protein standard; “+” represents a protein incubated with Compound 2-containing beads; “−” represents a protein incubated with blank beads.

FIGS. 6A-6D depict the characterization of the binding of Compounds I and II to the L-type calcium channel beta subunits.

FIG. 6A depicts Western analysis of fractions for the presence of CACNB 1 using the corresponding antibody.

FIG. 6B are bar graphs depicting the results from size exclusion chromatography to measure the fraction of [¹⁴C]-1 that binds to the purified recombinant CACBN1 and CACBN4.

FIG. 6C depicts the results of fluorescent analysis to measure the fluorescent quenching induced upon binding of Compound 2 (1 μM) to CACNB1 (0-8 μM).

FIG. 6D depicts the results of affinity chromatography to test the binding of CACNB1 to Compound 2. Lanes were labeled as follows: “C” represent a protein standard; “+” represents a protein incubated with Compound 2-containing beads; “−” represents a protein incubated with blank beads.

FIG. 7 provides an immunoblot of the co-immunoprecipitate of the lysate of F11 cells exposed to various concentrations of the rapamycin analogue I (0 μM, 5 μM or 50 μM) precipitated using an anti-FKBP52 antibody. The immunoprecipitated fractions were immunoblotted with an anti-Ca²⁺ channel β₁-subunit antibody. The lower panels provide diagrams summarizing the protein interactions. “RA I” represents rapamycin analogue I.

FIG. 8 provides a bar graph depicting the effect of various concentrations of rapamycin analogue I (50 μM, 5 μM, or 0 μM) on neurite outgrowth of F11 cells using neurofilament ELISA.

FIGS. 9A-9F depict the biological effect of Compounds 1 and 2 on calcium currents.

FIG. 9A is a bar graph of the mean Ca²⁺ current density from whole-cell recording in F-11 cells treated with 5 μM of Compound 1, FK-506 or vehicle in the bath for 2 hrs. Recordings were performed from 7 cells in each condition.

FIG. 9B depicts representative Ca²⁺ currents with internally applied Compound 1 (10 μM in pipette) at time 0 sec (bottom trace), 800 sec (middle trace) and in the presence of the L-type Ca²⁺ channel blocker BAY-K 5552 (top trace) externally.

FIG. 9C depicts a graph of the time course of the experiment illustrated in FIG. 9B. Whole cell, and subsequent diffusion of Compound 1 into the cell, begins at time 0. Once current stabilizes after 400 sec, 10 μM BayK-5552 is applied in the bath. (n=3)

FIG. 9D depicts similar conditions as in FIG. 9C, except that after 300 sec 100 nM ωCTX MVIIA is applied via the bath. (n=2)

FIG. 9E depicts the Ca²⁺ current trace from hippocampal neuron immediately upon break-in to whole-cell (control) and after 10 minutes of recording with 10 μM Compound 2 internally and ωCTX GVIA externally.

FIG. 9F depicts the mean responses (+/−SEM) normalized to the initial current from hippocampal neurons. Compound 2 (10 μM) applied internally via the recording pipette, beginning at time 0, where indicated ( and ▾). External solution contains 1 μM TTX+100 nM ωCTX GVIA+10 μM BAY-K 5552 (▾, n=4) or 1 μM TTX+100 nM ωCTX GVIA (, n=5). Control without compound (▪) contained 100 nM ωCTX GVIA externally (n=3).

FIG. 10A provides a graph demonstrating the effect of siRNA-driven reduction of FKBP52 and CACNB1 on neurite outgrowth.

FIG. 10B provides a graph demonstrating the effect of siRNA-driven reduction of FKBP52 and CACNB1 on neuronal survival.

FIG. 10C shows Western blots confirming that siRNA treatment reduced lamin A/C, CACNB1 or FKBP52 protein expression in cortical neurons after 24 hours.

FIGS. 11A-11B provide the amino acid sequence and nucleotide sequence of human Ca²⁺ channel β₁ subunit isoform 1 (SEQ ID NOs: 1-2, respectively).

FIGS. 11C-11D provide the amino acid and nucleotide sequence of human Ca²⁺ channel β₁ subunit isoform 2 (SEQ ID NOs: 3-4).

FIGS. 11E-11F provide the amino acid and nucleotide sequence of human Ca²⁺ channel β₁ subunit isoform 3 (SEQ ID NOs: 5-6).

FIGS. 11G-11H provide the amino acid and nucleotide sequence of a mouse (Mus musculus) Ca²⁺ channel β₁ subunit isoform A (SEQ ID NOs: 7-8).

FIG. 11I-11J provide the amino acid sequence of a mouse (Mus musculus) Ca²⁺ channel β₁ subunit isoform B (SEQ ID NOs: 9-10).

FIGS. 12A-12B provide the amino acid and nucleotide sequence of human FKBP52 (SEQ ID NOs:11-12).

FIGS. 12C-12D provide the amino acid sequence of mouse (Mus musculus) FKBP52 (SEQ ID NOs:13-14).

### DETAILED DESCRIPTION

The present invention is based, at least in part, on the discovery that immunophilin ligands, e.g., a rapamycin analogues modified at the mTOR binding region, interact with, e.g., bind to, the immunophilin FKBP52 and/or the voltage gated L-type calcium channel β1 subunit. Inhibition of FKBP52 and/or CACNB1 by these compounds stimulates neurite outgrowth and/or neuronal survival. Thus, interaction (and complex formation) between these components is believed to inhibit the activity of the β1 subunit and stimulate neurite outgrowth, implicating voltage gated L-type calcium channels in some of the neurotrophic and/or neuroprotective activities exhibited by immunophilin ligands, such as the rapamycin or meridamycin analogues described herein.

Applicants have additionally shown in the appended Examples that at least one of the immunophilin ligands disclosed herein (rapamycin analogue II) showed a significant increase in binding selectivity for FKBP52, relative to FKBP12 binding, of at least 600 fold higher compared to rapamycin. Without being bound by theory, it is believed that inhibition of FKBP52 activity mediates neurite outgrowth, presumably by activating steroid, e.g., glucocorticoid receptors. Furthermore, treatment of cortical neurons with the immunophilin ligands disclosed herein caused an overall downregulation of calcium signaling pathways and partial inhibition of L-type calcium channels. A significant effect on neurite outgrowth of neuronal cells was also detected by selectively reducing the expression of the β1 subunit and FKBP52 in culture.

The data disclosed herein demonstrate that modification of rapamycin at the mTOR binding region can provide significantly non-immunosuppressive compounds with unusual selectivity for FKBP52 and potent neurotrophic activities. FKBP52 appears to mediate immunophilin ligand-mediated neurite outgrowth, presumably by the activation of steroid receptors (including glucocorticoid receptors), as demonstrated by neurite outgrowth observed in FKBP52 siRNA treated cortical neurons. Further, the ability of these rapamycin analogues to partially inhibit L-type Ca²⁺ channels and reduce transcription of various Ca²⁺ signaling proteins indicates that these analogues can protect neurons from Ca²⁺ induced neuronal cell death, which is consistent with their effect on neuronal survival.

Calcium channels are present in various tissues, including neuronal and cardiovascular tissues, and have important roles in a number of vital processes in animals, including neurotransmitter release, muscle contraction, pacemaker activity, and secretion of hormones and other substances. Entry of calcium into neuronal cells through voltage-gated calcium channels mediates a wide variety of cellular and physiological responses, including, but not limited to, modulating the activity of calcium-dependent enzymes such as protein kinase C and calmodulin-dependent protein kinase II; controlling membrane potential and contributing to electrical properties such as excitability and repetitive firing patterns; and increasing neurotransmitter release. These processes, are involved in human disorders, such as neurological and cardiovascular disorders. Therefore, methods of inhibiting the function of voltage-dependent calcium channels by forming immunophilin-calcium channel complexes are useful for treating, preventing and/or alleviating symptoms of calcium channel disorders, as described in more detail herein.

In order that the present invention may be more readily understood, certain terms are described in more detail herein and throughout the detailed description.

Calcium channels are membrane-spanning, multi-subunit proteins that allow controlled entry of Ca²⁺ ions into cells from the extracellular fluid. The most common type of calcium channel is voltage dependent. “Excitable” cells in animals, such as neurons of the central nervous system (CNS), peripheral nerve cells, and muscle cells (including those of skeletal muscles, cardiac muscles, and venous and arterial smooth muscles) have voltage-dependent calcium channels. Voltage-gated calcium channels allow for influx of Ca²⁺ ions into a cell, and typically require a depolarization to a certain level of the potential difference between the inside of the cell bearing the channel and the extracellular environment bathing the cell. Voltage-gated calcium channels have been classified by their electrophysiological and pharmacological properties into L-, N-, P/Q-, R- and T-types (reviewed in Catterall, 2000; Huguenard 1996; Dolphin, A. C. (2003) Pharmacological Reviews 55:607-627). The L-, N- and P/Q-type channels activate at positive potentials (high voltage-gated). T-type (or low voltage-gated) channels describe a broad class of molecules that transiently activate at negative potentials and are highly sensitive to changes in resting potential.

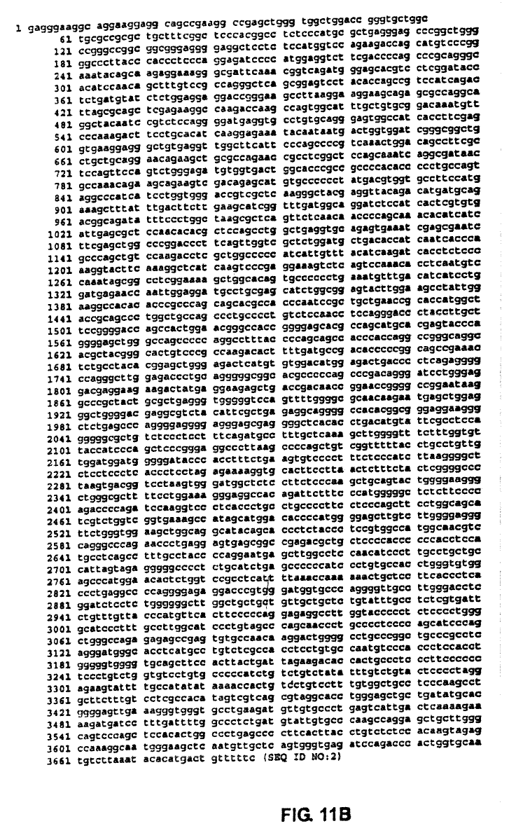

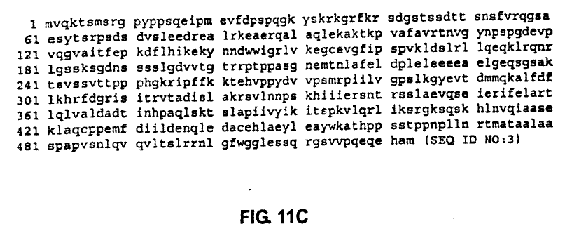

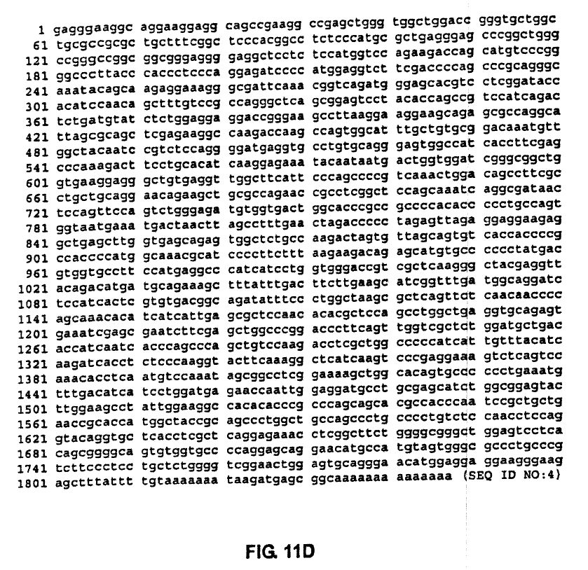

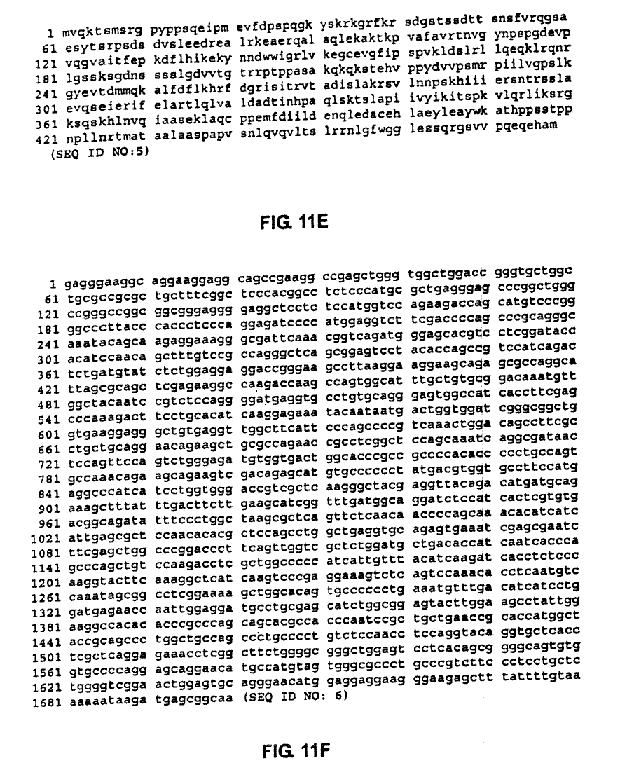

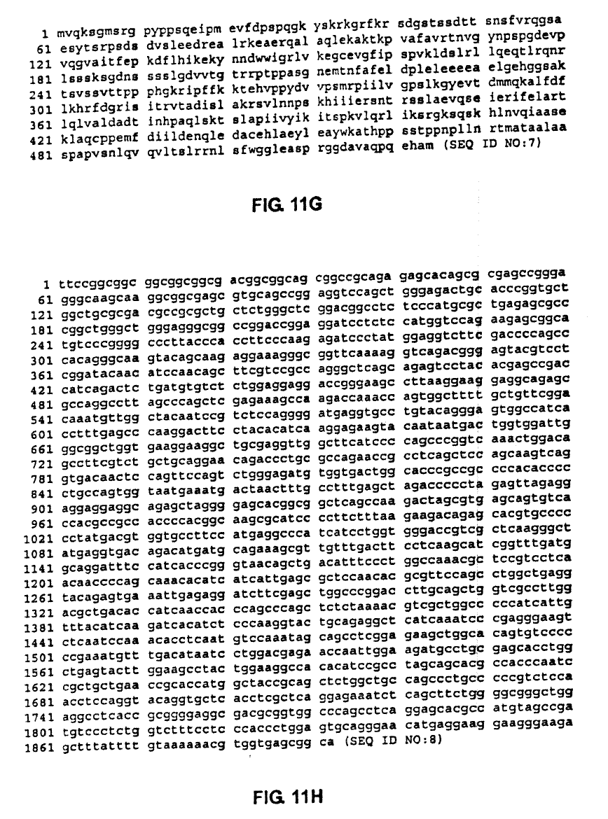

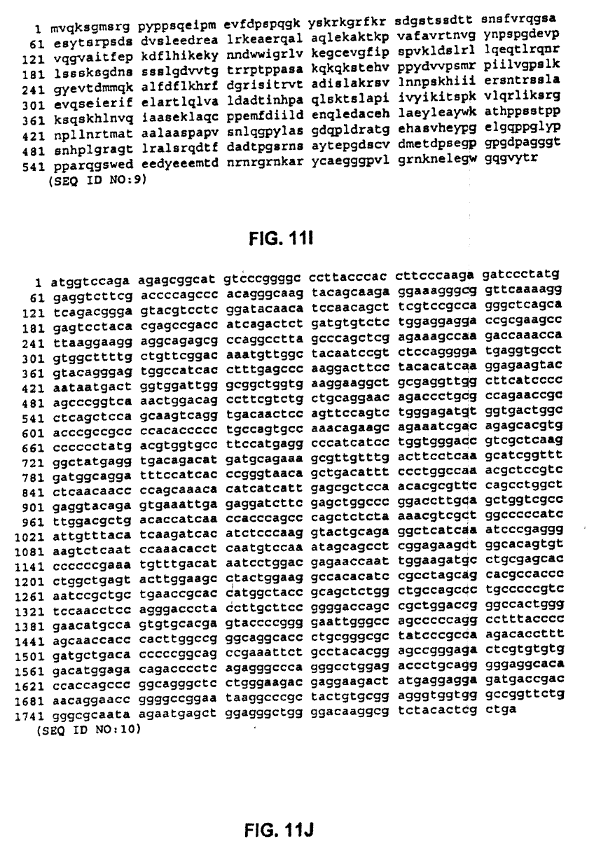

High voltage-gated calcium channels are composed of four distinct polypeptides: α₁, α₂δ, β and γ (reviewed by Stea et al., 1994; Catterall, 2000). The β subunit (also referred to herein as “CACB1”) is a soluble intracellular protein encoded by at least four known separate genes, each of which is processed into multiple splice variants. In embodiments, the β subunit has one or more of the following features: (i) an amino acid sequence of a naturally occurring mammalian (e.g., human or rodent) subunit or a fragment thereof, e.g., the amino acid sequence as shown in FIGS. 11A-11J (SEQ ID NOs:1-10) or a fragment thereof; (ii) an amino acid sequence substantially homologous to the amino acid sequence shown in FIGS. 11A-11J (SEQ ID NOs:1-10) or a fragment thereof; (iii) an amino acid sequence that is encoded by a naturally occurring mammalian (e.g., human or rodent) (β1 subunit nucleotide sequence or a fragment thereof, e.g., an amino acid sequence encoded by the nucleotide sequence as shown in FIGS. 11A-11J (SEQ ID NOs:1-10) or a fragment thereof; (iv) an amino acid sequence encoded by a nucleotide sequence which is substantially homologous to the nucleotide sequence shown in FIGS. 11A-11J (SEQ ID NOs:1-10) or a fragment thereof; (v) an amino acid sequence encoded by a nucleotide sequence degenerate to a naturally occurring β1 subunit nucleotide sequence or a fragment thereof, e.g., the nucleotide sequence shown in FIGS. 11A-11J (SEQ ID NOs:1-10) or a fragment thereof; or (vi) a nucleotide sequence that hybridizes to one of the foregoing nucleotide sequences under stringent conditions, e.g., highly stringent conditions. In some embodiments, the β subunit or functional variant (e.g., fragment) thereof exhibits one or more activities of the naturally-occurring sequence, including but not limited to, (i) forms a complex as described herein; (ii) interacts with, e.g., binds to, the α-subunit; (iii) facilitates the localization or trafficking of the voltage-gated calcium channel, e.g., the α₁ subunit, to the cellular plasma membrane; (iv) modulates gating of the channel (e.g., alters activation and inactivation kinetics, causes a leftward shift in the I-V curve and, at a single channel level, induces an increase in the channel opening probability); or (v) controls transcriptional activity of one or more of the genes described herein (e.g., calcium- influx channels, NMDA receptors, plasminogen activator (PLAU), SHT3R channels).

In other embodiments, the β subunit has a sequence substantially identical to that disclosed in Powers et al. (1992) J. Biol. Chem. 267(32):22967-22972; Collin et al. (1993) Circ. Res. 72(6):1337-1344; Hogan, K. et al. (1999) Neurosci. Lett. 277 (2), 111-114; Foell et al. (2004) Physiol. Genomics 17 (2), 183-200 (human 131 and (32 subunits); Toba et al. (2005) Eur. J. Neurosci. 22 (1), 79-92 (murine beta 1 subunit isoform); Serikov et al. (2002) Biochem. Biophys. Res. Commun. 293 (5), 1405-1411; Pragnell et al. (1991) FEBS Lett. 291 (2), 253-258; Cahill et al. (2000)J. Neurosci. 20 (5), 1685-1693 (2000) (bovine beta 1, 2 and 3 subunits); Rosenfeld et al. (1993) Ann. Neurol. 33 (1), 113-120; Taviaux et al. (1997) Hum. Genet. 100 (2), 151-154 (human genes for beta 2 and beta 4 subunits); Colecraft et al. (2002) J. Physiol. (Lond.) 541 (Pt 2), 435-452 (human beta 2a, 2c, 2d and 2e subunits); Opatowsky et al. (2003) J. Biol. Chem. 278 (52), 52323-52332 (rat beta 2 subunit); Yamada et al. (2001), J. Biol. Chem. 276 (50), 47163-47170 (2001) (rat beta 2 subunit); Strausberg et al. (2002) PNAS U.S.A. 99 (26), 16899-16903 (human beta 3 subunit, murine beta 4 subunit); Murakami et al. (1996) Eur. J. Biochem. 236 (1), 138-143 (1996) (murine calcium channel beta 3 subunit); Yamada et al. (1995) Genomics 27 (2), 312-319 (human calcium channel alpha 1 subunit (CACNL1A2) and beta subunit (CACNLB3) genes); Chen et al. (2004) Nature 429 (6992), 675-680 (human beta 4 subunit); Helton et al. (2002) J. Neurosci. 22 (5), 1573-1582 (2002) (beta 4 subunit); Badou et al. (2005) Science 307 (5706), 117-121 (2005) (calcium channel beta4 subunit); the contents of all of which are hereby incorporated by reference. Other β subunit sequences are disclosed in Genbank Accession Numbers: NP_{—} 666235, Q9Y698, Q02641, Q9MZL3 and P54288_{—}2.

Immunophilins are soluble cytosolic proteins that form complexes with immunophilin ligands, which in turn serve as ligands for other cellular targets involved in signal transduction. Classes of immunophilins include cyclophilins and FK506-binding proteins (e.g., FKBPs), such as FKBP-12 and FBBP-52. Cyclosporin A is a macrolide immunophilin ligand that binds to cyclophilins. Other macrolide immunophilin ligands, such as meridamycin, FK506, FK520, and rapamycin, are understood to bind to FKBPs. Binding of FK506, FK520 and rapamycin to FKBP typically occurs through structurally similar segments of the polyketide molecules, referred to as “FKBP-binding domain.”

Gene sequences corresponding to more than two-dozen FKBPs have been found in the human genome (Dornan et al., Curr. Top. Med. Chem. 3, 1392-1409 (2003)). They are expressed 10-50 fold higher in central nervous system (CNS) and peripheral nervous system (PNS) tissue than in immune tissue (Lyons et al., J. Neurosci. 15, 2985-2994 (1995)), and their expression is increased following the onset of neurological disease (Kihira et al., Neuropathology 22, 269-274 (2002)). Interestingly, FKBP12, FKBP12.6 and FKBP52 were reported as channel-gating-FKBP proteins, modulating ryanodine receptor (RYR) (Huang et al., Proc. Natl. Acad. Sci. USA. 103, 3456-3461 (2006)), inositol 1,4,5-trisphosphate receptor (IP₃R) (Cameron et al., Proc. Natl. Acad. Sci. USA. 92, 1784-1788 (1995)) and transient receptor potential channels (TRPC) (Sinkins et al., J. Biol. Chem. 279, 34521-34529 (2004)). FKBP52 and FKBP51 associate with three types of steroid receptor complexes that mediate the down-stream responses to estrogen, androgen and glucocorticoid hormones (Steiner et al., Proc. Natl. Acad. Sci. USA. 94, 2019-2024 (1997)). The nuclear FKBP25 regulates gene expression through associating with histone deacetylase, casein kinase II, nucleolin and transcription factor YY1 (Yao and Yang, Curr. Cancer Drug Targets 5, 595-610 (2005)). FKBP38 is constitutively inactive and located at the mitochondria and endoplasmic reticulum. Interestingly, high levels of Ca²⁺ and calmodulin (CaM) are required for FKBP38 to bind Bcl-2 (Edlich et al., EMBO J. 24, 2688-2699 (2005)). Immunophilin ligands cause various down-stream biological activities by disruption of the natural FKBP-containing complexes (Gold Drug Metab. Rev. 31, 649-663 (1999); Edlich et al., J. Biol. Chem. 281, 14961-14970 (2006)) and by formation of novel complexes, such as FKBP12-FK506-calcineurin or FKBP12-rapamycin-mammalian target of rapamycin (mTOR) (Kissinger et al., Nature 378, 641-644 (1995); Choi et al., Science 273, 239-42 (1996)).

FKBP52 is a member of the FK506-binding class of immunophilins. Binding of FK506 to the glucocoricoid receptor (GR)-associated FKBP52 caused increased nuclear translocation of GR in response to dexamethasone and potentiation of GR-mediated gene expression (Sanchez and Ning (1996) Methods: A Companion to Meth. Enzymol. 9:188-200). Immunophilins such as FKBP52 and CyP40 and non- immunophilin proteins such as PP5, p60, and Mas70p, have one or more tetratricopeptide repeat (TPR) domains (Ratajczak et al. (1993) J. Biol. Chem. 268:13187-13192) that bind to the TPR-binding domain of hsp90. The number of TPR domains in a protein appears to correlate with its hsp90-binding affinity. Regions bordering the TPR domain also participate in binding, e.g., residues 232-271 of FKBP52 (Ratajczak and Carrello (1996) supra).

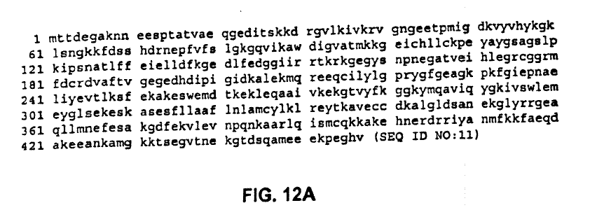

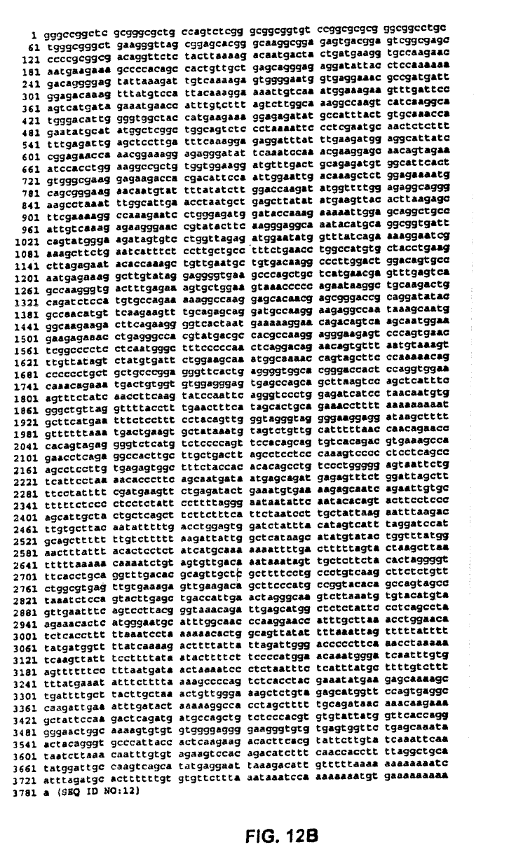

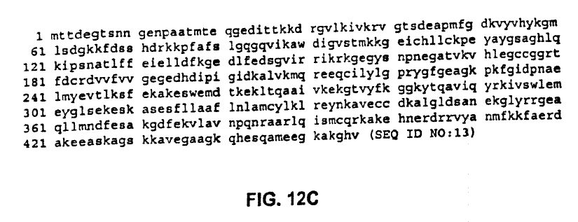

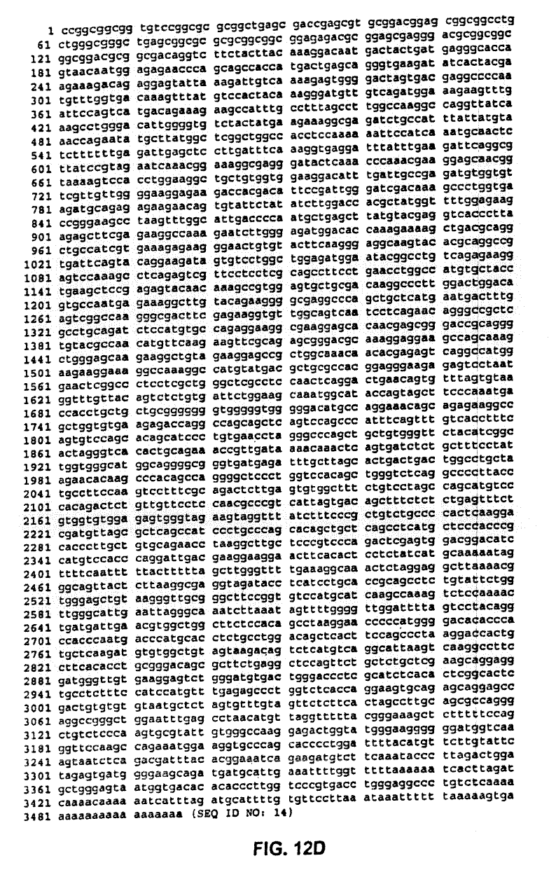

In some embodiments, the immunophilin has one or more of the following features: (i) an amino acid sequence of a naturally occurring mammalian (e.g., human or rodent) FKBP52 or a fragment thereof, e.g., the amino acid sequence as shown in FIGS. 12A-12D (SEQ ID NOs:11-14) or a fragment thereof; (ii) an amino acid sequence substantially homologous to the amino acid sequence shown in FIGS. 12A-12D (SEQ ID NOs:11-14) or a fragment thereof; (iii) an amino acid sequence that is encoded by a naturally occurring mammalian (e.g., human or rodent) FKBP52 nucleotide sequence or a fragment thereof, e.g., an amino acid sequence that is encoded by the nucleotide sequence as shown in FIGS. 12A-12D (SEQ ID NOs:11-14) or a fragment thereof; (iv) an amino acid sequence encoded by a nucleotide sequence which is substantially homologous to the nucleotide sequence shown in FIGS. 12A-12D (SEQ ID NOs:11-14) or a fragment thereof; (v) an amino acid sequence encoded by a nucleotide sequence degenerate to a naturally occurring FKBP52 nucleotide sequence or a fragment thereof, e.g., the nucleotide sequence shown in FIGS. 12A-12D (SEQ ID NOs:11-14) or a fragment thereof; or (vi) a nucleotide sequence that hybridizes to one of the foregoing nucleotide sequences under stringent conditions, e.g., highly stringent conditions. In some embodiments, the FKBP52 or functional variant (e.g., fragment) thereof exhibits one or more activities of the naturally-occurring sequence, including but not limited to, forms a complex as described herein; binds to FK506; increases nuclear translocation of a glucocorticoid receptor in response to dexamethasone; potentiates glucocorticoid receptor -mediated gene expression; and/or binds to a heat shock protein, e.g., hsp90.

Exemplary amino acid and nucleotide sequences for FKBP52 are disclosed in Sanchez et al. (1990) Biochemistry 29 (21), 5145-5152; and Peattie et al. (1992) Proc. Natl. Acad. Sci. U.S.A. 89 (22), 10974-10978, the contents of both of which are hereby incorporated by reference.

In one embodiment, β subunit or immunophilin polypeptides of this invention include, but are not limited to, fragments of native polypeptides from any animal species (including humans, rodents), and variants (e.g., functional variants) thereof (human and non-human) polypeptides and their fragments, provided that they have a biological activity in common with a respective native polypeptide. “Fragments” comprise, in one embodiment, regions within the sequence of a mature native polypeptide. Any form of the β subunit or immunophilin, e.g., FKBP52, of less than full length can be used in the methods and compositions of the present invention, provided that it is still functional, e.g., retains at least one activity of the naturally-occurring sequence (e.g., retains the ability to form a complex as described herein). β subunits of less than full length can be produced by expressing a corresponding fragment of the polynucleotide encoding the full-length β subunit protein in a host cell. These corresponding polynucleotide fragments are also part of the present invention. Modified polynucleotides as described above may be made by standard molecular biology techniques, including construction of appropriate desired deletion mutants, site-directed mutagenesis methods or by the polymerase chain reaction using appropriate oligonucleotide primers.

A “variant” of a polypeptide, or fragment thereof, such as, for example, a variant of a β1 subunit or FKBP52 includes chimeric proteins, labeled proteins (e.g., radiolabeled proteins), fusion proteins, mutant proteins, proteins having similar (e.g., substantially similar) sequences (e.g., proteins having amino acid substitutions (e.g., conserved amino acid substitutions), deletions, insertions), protein fragments, mimetics, so long as the variant has at least a portion of an amino acid sequence of a native protein, or at least a portion of an amino acid sequence of substantial sequence identity to the native protein. A “functional variant” includes a variant that retains at least one function of the native protein, e.g., retains the ability to interact an immunophilin ligand with and/or form a complex as described herein.

A “chimeric protein” or “fusion protein” is a fusion of a first amino acid sequence encoding a polypeptide with a second amino acid sequence, wherein the first and second amino acid sequences do not occur naturally as part of a single polypeptide chain.

As used herein, the term “substantially similar” (or “substantially” or “sufficiently” “homologous” or “identical”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. Sequences similar or homologous (e.g., at least about 85% sequence identity) to the sequences disclosed herein are also part of this application. In some embodiments, the sequence identity can be about 90%, 91%, 92%, 93%, 94%, 95%, 96%, 97%, 98%, 99% or higher. Alternatively, substantial identity exists when the nucleic acid segments hybridizes under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). Typically, the length of a reference sequence aligned for comparison purposes is at least 30%, preferably at least 40%, more preferably at least 50%, even more preferably at lo least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch ((1970) J. Mol. Biol. 48:444-453) algorithm which has been incorporated into the commercially available GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the commercially available GAP program in the GCG software package, using a NWSgapdna.CMP matrix and a gap weight of 40, 50, 60, 30 70, or 80 and a length weight of 1, 2, 3, 4, 5, or 6. Parameters typically used to determine percent homology are a Blossum 62 scoring matrix with a gap penalty of 12, a gap extend penalty of 4, and a frameshift gap penalty of 5. The percent identity between two amino acid or nucleotide sequences can also be determined using the s algorithm of E. Meyers and W. Miller ((1989) CABIOS 4:11-17) which has been incorporated into the ALIGN program (version 2.0), using a PAM120 weight residue table, a gap length penalty of 12 and a gap penalty of 4.

As used herein, the term “hybridizes under stringent conditions” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and non-aqueous methods are described in that reference and either can be used. An example of stringent hybridization conditions are hybridization in 6× sodium chloride/sodium citrate (SSC) at about 45° C., followed by one or more washes in 0.2×SSC, 0.1% SDS at 50° C. Another is example of stringent hybridization conditions are hybridization in 6×SSC at about 45° C., followed by one or more washes in 0.2×SSC, 0.1% SDS at 55° C. A further example of stringent hybridization conditions are hybridization in 6×SSC at about 45° C., followed by one or more washes in 0.2×SSC, 0.1% SDS at 60° C. Typically, stringent hybridization conditions are hybridization in 6×SSC at about 45° C., followed by one or 20 more washes in 0.2×SSC, 0.1% SDS at 65° C. More typically, the highly stringent conditions used are 0.5M sodium phosphate, 7% SDS at 65° C., followed by one or more washes at 0. 2×SSC, 1% SDS at 65° C.

It is understood that the variants of the polypeptide disclosed herein may have additional conservative or non-essential amino acid substitutions, which do not have a substantial effect on antigen binding or other immunoglobulin functions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, praline, phenylalanine, methionine, tryptophan), beta-branched side chains (e.g., threonine, valine, isoleucine) and aromatic side chains (e.g., s tyrosine, phenylalanine, tryptophan, histidine).

A “non-essential” amino acid residue is a residue that can be altered from the wild-type sequence of a hybrid antibody, without abolishing or more preferably, without substantially altering a biological activity, whereas an “essential” amino acid residue results in such a change.

### Immunophilin Ligands

Immunophilin ligands bind to immunophilins to activate other cellular targets, primarily in the immune and nervous system. Several immunophilins are immunosuppressive, e.g., cyclosporin A, FK506 and rapamycin, whereas other less immunosuppressive immunophilins show neurotrophic activities. For example, meridamycin is substantially non-immunosuppressive and shows significant neuroprotective activity in vitro (US 2005/0272133 by He, M. et al. published on Dec. 8, 2005, and US 2005/0197356 by Graziani, E. et al. published on Sep. 8, 2005). Preferably, immunophilin ligands identified by, or used in, the methods of the invention are substantially non-immunosuppressive, but retain a desirable activity, e.g., a neurotrophic activity. Preferred immunophilin ligands increase the formation of a complex as described herein and/or reduce FKBP and/or calcium channel activity.

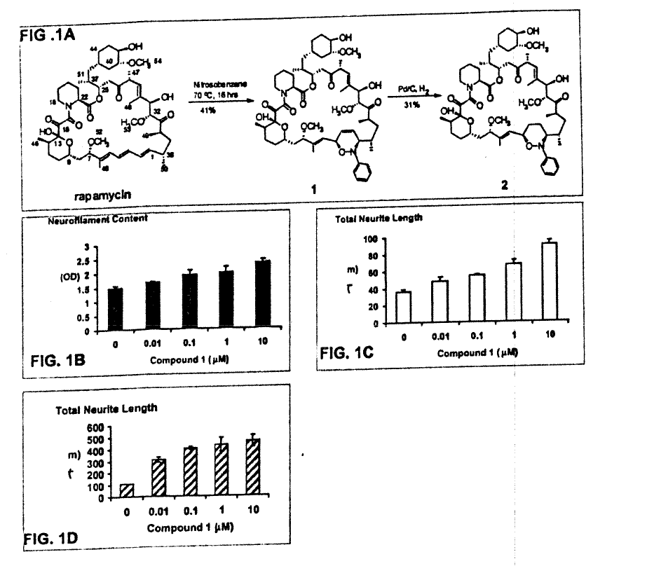

In some embodiments, the immunophilin ligands are modified at the mTOR binding domain. The mTOR binding domain of rapamycin is believed to localize at the macrocycle core at about positions 1-7 and 27-36 of FIG. 1A. For example, the immunophilin ligands can have a heteroatom substituent at positions 1 and 4 of the rapamycin backbone (FIG. 1A). In other embodiments, the rapamycin analogues have a cyclic structure at positions 1, 2, 3 and/or 4 (FIG. 1A). Such rapamycin analogues are disclosed in commonly assigned co-pending published application U.S. 2006/0135549 entitled “Rapamycin Analogues and the Uses Thereof in the Treatment of Neurological, Proliferative, and Inflammatory Disorders,” published on Jun. 22, 2006 from U.S. Ser. No. 11/300,839, the entire content of which is hereby incorporated by reference.

In one embodiment, the rapamycin analogues have the formula I:

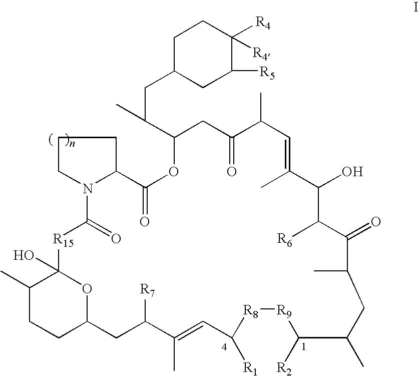

R₁ and R₂ in the above-noted formula are different, independent groups and are selected from among OR₃ and N(R_{3′})(R_{3″}) or R₁ and R₂ are different, are connected through a single bond, and are selected from O and NR₃. R₃, R_{3′}, and R_{3″} are independently selected from among H, C₁ to C₆ alkyl, C₁ to C₆ substituted alkyl, C₃ to C₈ cycloalkyl, substituted C₃ to C₈ cycloalkyl, aryl, substituted aryl, heteroaryl, and substituted heteroaryl. R₄ and R_{4′} are (a) independently selected from among H, OH, O(C₁ to C₆ alkyl), O(substituted C₁ to C₆ alkyl), O(acyl), O(aryl), O(substituted aryl), and halogen; or (b) taken together to form a double bond to O. R₅, R₆, and R₇ are independently selected from among H, OH, and OCH₃. R₈ and R₉ are connected through a (i) single bond and are CH₂ or (ii) double bond and are CH. R₁₅ is selected from among C═O, CHOH, and CH₂ and n is 1 or 2; or pharmaceutically acceptable, salts, prodrugs, or metabolites thereof.

In further embodiments, R₁ and R₂ are connected through a single bond and are selected from O and NR₃. In still a further embodiment, R₁ is O and R₂ is NR₃.

In one embodiment, R_{3′} or R_{3″} is an aryl or substituted aryl group, or a substituted benzene ring. In another embodiment, substituted benzene groups at R_{3′} or R_{3″} include rings of the following structure:

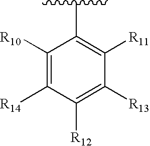

R₁₀, R₁₁, R₁₂, R₁₃, and R₁₄ are independently selected from among H, C₁ to C₆ alkyl, substituted C₁ to C₆ alkyl, aryl, substituted aryl, heteroaryl, substituted heteroaryl, halogen, acyl, OH, O(alkyl), O(substituted alkyl), O(aryl), O(substituted aryl), O(acyl), NH₂, NH(alkyl), NH(substituted alkyl), NH(aryl), NH(substituted aryl), and NH(acyl).

In further embodiments, R₃, R_{3′} or R_{3″} are phenyl optionally substituted by 1 or 2 substituents selected from C₁ to C₆ alkyl and halogen. In still further embodiments, R₃, R_{3′} or R_{3″} are phenyl optionally substituted with 1 or 2 methyl or chloro substituents, e.g. phenyl and 3-methyl, 4-chlorophenyl.

In one embodiment, R₄ or R_{4′} are OH or O(acyl), e.g., where the acyl is

—C(O)— optionally substituted alkyl, in particular where alkyl can be straight or branched and optionally substituted e.g. by heterocyclic such as aromatic heterocyclic such as pyridyl. An example is:

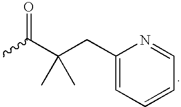

In other embodiments, rapamycin analogues of formula I include those where R₅, R₆ and R₇ are OCH₃, those where the nitrogen containing ring at positions 17-22 of the rapamycin backbone is a piperidine ring, or where R₁₅ is a carbonyl.

In one embodiment, the rapamycin analogues have the formula Ia:

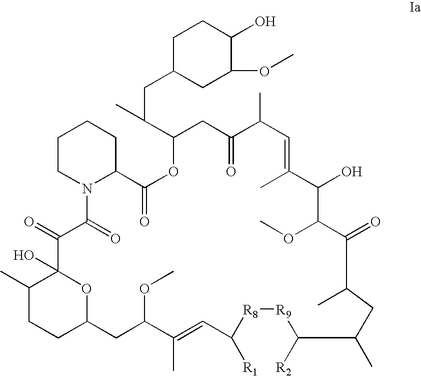

where R₁, R₂, R₃, and R₉ are defined as noted above.

In another embodiment, the rapamycin analogues have the following formula Ib:

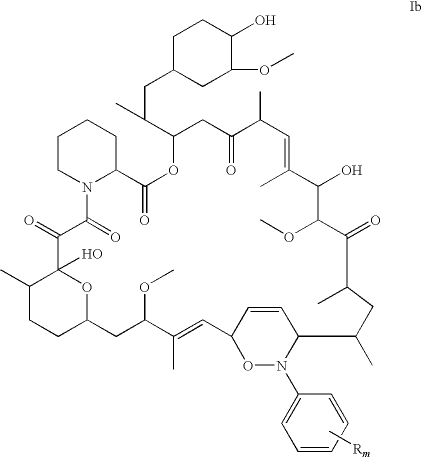

In formula Ib, R is independently selected from among H, C₁ to C₆ alkyl, substituted C₁ to C₆ alkyl, aryl, substituted aryl, heteroaryl, substituted heteroaryl, halogen, acyl, OH, O(alkyl), O(substituted alkyl), O(aryl), O(substituted aryl), O(acyl), NH₂, NH(alkyl), NH(substituted alkyl), NH(aryl), NH(substituted aryl), and NH(acyl) and m is 1 to 5.

Specific rapamycin analogues are illustrated herein and include 9,27-dihydroxy-3-{2-[4-hydroxy-3-methoxycyclohexyl ]-1-methylethyl}-10,21-dimethoxy-6,8,12,14,20,26-hexamethyl-37-phenyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H, 31H)-pentone; 9,27-dihydroxy-3-{2-[4-hydroxy-3-methoxycyclohexyl]-1-methylethyl}-10,21-dimethoxy-6,8,12,14,20,26-hexamethyl-37-phenyl-4,9,10,12,13,14,15,16,17,18,21,22,23,24,25,26,27,32,33,34,34a-henicosahydro-3H-23,27-epoxy-18,15-(epoxyimino)pyrido[2,1-c][1,4]oxazacyclohentriacontine-1,5,11,28,29(6H, 31H)-pentone; 37-(4-chloro-3-methylphenyl)-9,27-dihydroxy-3-{-2-[4-hydroxy-3-methoxycyclohexyl]-1-methylethyl}-10,21-dimethoxy-6,8,12,14,20,26-hexamethyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H, 31H)-pentone; 37-(2,6-dichlorophenyl)-9,27-dihydroxy-3-{2-[4-hydroxy-3-methoxycyclohexyl]-1-methylethyl}-10,21-dimethoxy-6,8,12,14,20,26-hexamethyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H, 31H)-pentone; 9,27-dihydroxy-3-{-2-[4-hydroxy-3-methoxycyclohexyl]-1-methylethyl}-10,21-dimethoxy-6,8,12,14,20,26-hexamethyl-37-phenyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H, 31H)-pentone ester with -2,2-dimethyl-3-(pyridin-2-yl)-propionic acid; 37-(2,6-dichlorophenyl)-9,27-dihydroxy-3-{-2-[4-hydroxy-3-methoxycyclohexyl]-1-methylethyl}-10,21-dimethoxy-6,8,12,14,20,26-hexamethyl-4,9,10,12,13,14,15,18,21,22,23,24,25,26,27,32,33,34,34a-nonadecahydro-3H-23,27-epoxy-18,15-(epoxyimino)pyrido[2,1-c][1,4]oxazacyclohentriacontine-1,5,11,28,29(6H, 31H)-pentone; or pharmaceutically acceptable, salts, prodrugs, or metabolites thereof. The invention is not limited to these illustrative compounds.

In another embodiment, the specific compounds include the following:

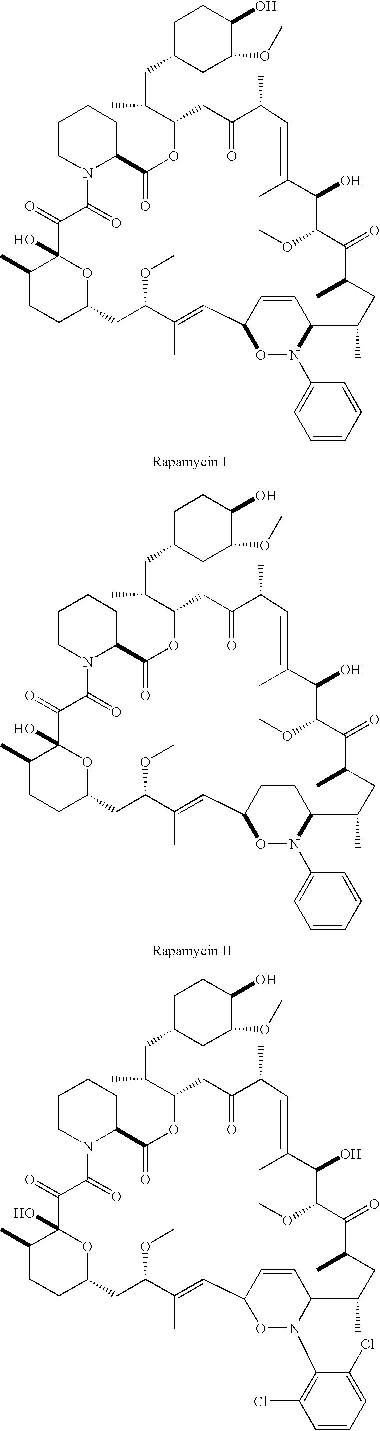

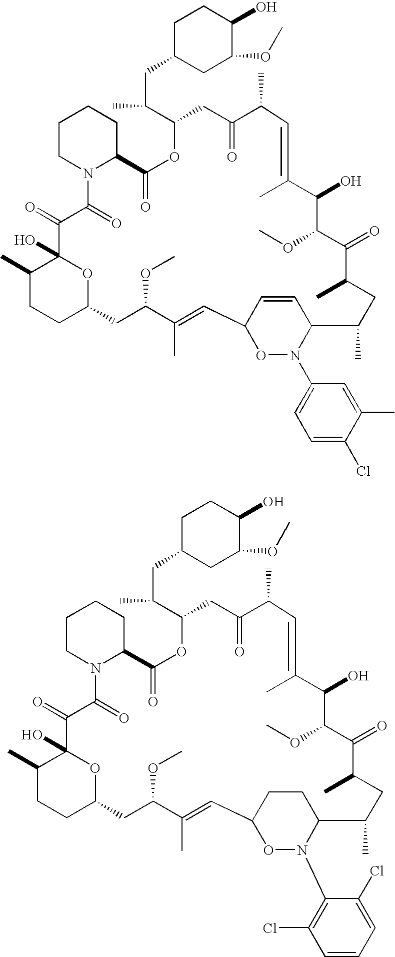

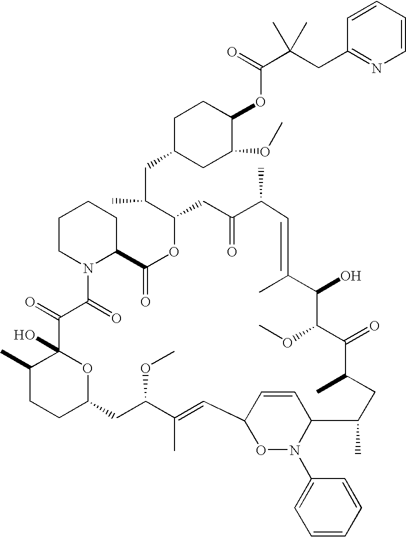

Rapamycin analogues I and II, referred to throughout the application, are represented by the first and second chemical structures, respectively, shown from the top left.

Rapamycin analogues also include compounds where R₁ and R₂ are connected through a single bond; R₁ is O; R₂ is NR₃; R₃ is phenyl; R₄ is OH; R₅-R₇ are OCH₃; and R₈ and R₉ are HC═CH; a compound where R₁ is OR₃; R₂ is N(R_{3′})(R_{3″}); R₃ is H; R_{3′} is H; R_{3″} is phenyl; R₄ is OH; R₅-R₇ are OCH₃; and R₈ and R₉ are H₂C—CH₂; a compound where R₁ and R₂ are connected through a single bond; R₁ is O; R₂ is NR₃; R₃ is phenyl; R₄ is OH; R₅-R₇ are OCH₃; and R₈ and R₉ are H₂C—CH₂; a compound where R₁ and R₂ are connected through a single bond; R₁ is O; R₂ is NR₃; R₄ is OH; R₅-R₇ are OCH₃; R₈ and R₉ are HC═CH; and R₃ is

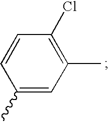

a compound where R₁ and R₂ are connected through a single bond; R₁ is O; R₂ is NR₃; R₄ is OH; R₅-R₇ are OCH₃; R₈ and R₉ are HC═CH; and R₃ is

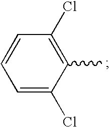

a compound where R₁ and R₂ are connected through a single bond; R₁ is O; R₂ is NR₃; R₃ is phenyl; R₅-R₇ are OCH₃; R₈ and R₉ are HC═CH; and R₄ is

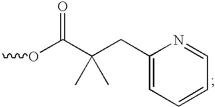

and a compound where R₁ and R₂ are connected through a single bond; R₁ is O; R₂ is NR₃; R₄ is OH; R₅-R₇ are OCH₃; R₈ and R₉ are H₂C—CH₂; and R₃ is

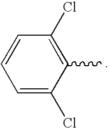

The compounds can contain one or more asymmetric carbon atoms and some of the compounds can contain one or more asymmetric (chiral) centers and can thus give rise to optical isomers and diastereomers. While shown without respect to stereochemistry, when the compounds can contain one or more chiral centers, preferably at least one of the chiral centers is of S-stereochemistry. Thus, the compound includes such optical isomers and diastereomers; as well as the racemic and resolved, enantiomerically pure stereoisomers; as well as other mixtures of the R and S stereoisomers, and pharmaceutically acceptable salts, hydrates, metabolites, and prodrugs thereof.

The term “alkyl” is used herein to refer to both straight- and branched-chain saturated aliphatic hydrocarbon groups having 1 to 10 carbon atoms, and desirably about 1 to 8 carbon atoms. The term “alkenyl” is used herein to refer to both straight- and branched-chain alkyl groups having one or more carbon-carbon double bonds and containing about 2 to 10 carbon atoms. In one embodiment, the term alkenyl refers to an alkyl group having 1 or 2 carbon-carbon double bonds and having 2 to about 6 carbon atoms. The term “alkynyl” group is used herein to refer to both straight- and branched-chain alkyl groups having one or more carbon-carbon triple bond and having 2 to 8 carbon atoms. In another embodiment, the term alkynyl refers to an alkyl group having 1 or 2 carbon-carbon triple bonds and having 2 to 6 carbon atoms.

The term “cycloalkyl” is used herein to refer to an alkyl group as previously described that is cyclic in structure and has about 4 to 10 carbon atoms, or about 5 to 8 carbon atoms.

The terms “substituted alkyl”, “substituted alkenyl”, and “substituted alkynyl” refer to alkyl, alkenyl, and alkynyl groups, respectively, having one or more substituents including, without limitation, halogen, CN, OH, NO₂, amino, aryl, heterocyclic, alkoxy, aryloxy, alkylcarbonyl, alkylcarboxy, and arylthio, which groups can be optionally substituted e.g. by 1 to 4 substituents including halogen, CN, OH, NO₂, amino, alkyl, cycloalkyl, alkenyl, alkynyl, alkoxy, aryloxy, alkyloxy, alkylcarbonyl, alkylcarboxy, aminoalkyl, and arylthio. These substituents can be attached to any carbon of an alkyl, alkenyl, or alkynyl group provided that the attachment constitutes a stable chemical moiety.

The term “aryl” as used herein refers to an aromatic system, e.g., of 6-20 carbon atoms, which can include a single ring or multiple aromatic rings fused or linked together (e.g. two or three) where at least one part of the fused or linked rings forms the conjugated aromatic system. The aryl groups can include, but are not limited to, phenyl, naphthyl, biphenyl, anthryl, tetrahydronaphthyl, phenanthryl, indene, benzonaphthyl, fluorenyl, and carbazolyl.

The term “substituted aryl” refers to an aryl group which is substituted with one or more substituents including halogen, CN, OH, NO₂, amino, alkyl, cycloalkyl, alkenyl, alkynyl, alkoxy, aryloxy, alkyloxy, alkylcarbonyl, alkylcarboxy, aminoalkyl, and arylthio, which groups can be optionally substituted. In one embodiment, a substituted aryl group is substituted with 1 to 4 substituents including halogen, CN, OH, NO₂, amino, alkyl, cycloalkyl, alkenyl, alkynyl, alkoxy, aryloxy, alkyloxy, alkylcarbonyl, alkylcarboxy, aminoalkyl, and arylthio.

The term “heterocyclic” as used herein refers to a stable 4- to 7-membered monocyclic or multicyclic heterocyclic ring which is saturated, partially unsaturated, or wholly unsaturated, including aromatic such as pyridyl. The heterocyclic ring has carbon atoms and one or more heteroatoms including nitrogen, oxygen, and sulfur atoms. In one embodiment, the heterocyclic ring has 1 to 4 heteroatoms in the backbone of the ring. When the heterocyclic ring contains nitrogen or sulfur atoms in the backbone of the ring, the nitrogen or sulfur atoms can be oxidized. The term “heterocyclic” also refers to multicyclic rings, e.g., of 9 to 20 ring members in which a heterocyclic ring is fused to an aryl ring. The heterocyclic ring can be attached to the aryl ring through a heteroatom or carbon atom, provided the resultant heterocyclic ring structure is chemically stable. A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Oxygen-containing rings include, but are not limited to, furyl, tetrahydrofuranyl, pyranyl, pyronyl, and dioxinyl rings. Nitrogen-containing rings include, without limitation, pyrrolyl, pyrazolyl, imidazolyl, triazolyl, pyridyl, piperidinyl, 2-oxopiperidinyl, pyridazinyl, pyrimidinyl, pyrazinyl, piperazinyl, azepinyl, triazinyl, pyrrolidinyl, and azepinyl rings. Sulfur-containing rings include, without limitation, thienyl and dithiolyl rings. Mixed heteroatom containing rings include, but are not limited to, oxathiolyl, oxazolyl, thiazolyl, oxadiazolyl, oxatriazolyl, dioxazolyl, oxathiazolyl, oxathiolyl, oxazinyl, oxathiazinyl, morpholinyl, thiamorpholinyl, thiamorpholinyl sulfoxide, oxepinyl, thiepinyl, and diazepinyl rings. Fused heteroatom-containing rings include, but are not limited to, benzofuranyl, thionapthene, indolyl, benazazolyl, purindinyl, pyranopyrrolyl, isoindazolyl, indoxazinyl, benzoxazolyl, anthranilyl, benzopyranyl, quinolinyl, isoquinolinyl, benzodiazonyl, naphthylridinyl, benzothienyl, pyridopyridinyl, benzoxazinyl, xanthenyl, acridinyl, and purinyl rings.

The term “substituted heterocyclic” as used herein refers to a heterocyclic group having one or more substituents including halogen, CN, OH, NO₂, amino, alkyl, cycloalkyl, alkenyl, alkynyl, alkoxy, aryloxy, alkyloxy, alkylcarbonyl, alkylcarboxy, aminoalkyl, and arylthio, which groups can be optionally substituted. In one embodiment, a substituted heterocyclic group is substituted with 1 to 4 substituents.

The term “acyl” refers to a —C(O)— group, which is substituted at the carbon atom. The acyl group can be substituted or a terminal acyl group such as an HC(O)— group. The substituents can include any substituents noted above for alkyl groups, viz. one or more substituents including, without limitation, halogen, CN, OH, NO₂, amino, aryl, heterocyclic, alkoxy, aryloxy, alkylcarbonyl, alkylcarboxy, and arylthio, which groups can be optionally substituted. Examples include —C(O)-alkoxy (e.g. —OMe or —OEt) or —C(O)-alkyl where alkyl can be straight or branched and optionally substituted e.g., by heterocyclic (such as pyridyl).

The term “alkoxy” as used herein refers to the O(alkyl) group, where the point of attachment is through the oxygen-atom and the alkyl group is optionally substituted.

The term “aryloxy” as used herein refers to the O(aryl) group, where the point of attachment is through the oxygen-atom and the aryl group is optionally substituted.

The term “alkyloxy” as used herein refers to the alkylOH group, where the point of attachment is through the alkyl group.

The term “arylthio” as used herein refers to the S(aryl) group, where the point of attachment is through the sulfur-atom and the aryl group can be optionally substituted.

The term “alkylcarbonyl” as used herein refers to the C(O)(alkyl) group, where the point of attachment is through the carbon-atom of the carbonyl moiety and the alkyl group is optionally substituted.

The term “alkylcarboxy” as used herein refers to the C(O)O(alkyl) group, where the point of attachment is through the carbon-atom of the carboxy moiety and the alkyl group is optionally substituted.

The term “aminoalkyl” as used herein refers to both secondary and tertiary amines where the point of attachment is through the nitrogen-atom and the alkyl groups are optionally substituted. The alkyl groups can be the same or different.

The term “halogen” as used herein refers to Cl, Br, F, or I groups.

The rapamycin analogues can be prepared from a rapamycin starting material. Preferably, the rapamycin starting material includes, without limitation, rapamycin, norrapamycin, deoxorapamycin, desmethylrapamycins, or desmethoxyrapamycin, or pharmaceutically acceptable salts, prodrugs, or metabolites thereof. However, one of skill in the art would readily be able to select a suitable rapamycin starting material that can be utilized to prepare the novel rapamycin analogues of the present invention.

The term “desmethylrapamycin” refers to the class of rapamycin compounds which lack one or more methyl groups. Examples of desmethylrapamycins that can be used according to the present invention include 29-desmethylrapamycin (U.S. Pat. No. 6,358,969), 7-O-desmethyl-rapamycin (U.S. Pat. No. 6,399,626), 17-desmethylrapamycin (U.S. Pat. No. 6,670,168), and 32-O-desmethylrapamycin, among others.

The term “desmethoxyrapamycin” refers to the class of rapamycin compounds which lack one or more methoxy groups and includes, without limitation, 32-desmethoxyrapamycin.

The rapamycin analogues can be prepared by combining a rapamycin starting material and a dienophile. The term “dienophile” refers to a molecule that reacts with a 1,3-diene to give a [4+2] cycloaddition product. Preferably, the dienophile utilized in the present invention is an optionally substituted nitrosobenzene. A variety of nitrosobenzenes can be utilized in the present invention and include nitrosobenzene, 2,6-dichloronitrosobenzene, and 1-chloro-2-methyl-4-nitrosobenzene, among others. One of skill in the art would readily be able to select the amount of nitrosobenzene that would be effective in preparing the rapamycin analogues of the present invention. Preferably, an excess of the nitrosobenzene is utilized, and more preferably in a 5:1 ratio of nitrosobenzene to rapamycin starting material. However, even a 1:1, 2:1, or 3:1 ratio of nitrosobenzene to rapamycin can be utilized as determined by one of skill in the art.

The nitrosobenzene and rapamycin starting material is combined in a solvent. The solvent preferably dissolves the nitrosobenzene and/or rapamycin on contact, or dissolves the nitrosobenzene and rapamycin as the reaction proceeds. Solvents that can be utilized in the present invention include, without limitation, dimethylformamide, dioxane such as p-dioxane, chloroform, alcohols such as methanol and ethanol, ethyl acetate, water, acetonitrile, tetrahydrofuran, dichloromethane, and toluene, or combinations thereof. However, one of skill in the art would readily be able to select a suitable solvent based upon the solubility of the rapamycin starting material and nitrosobenzene, as well as the reactivity of the solvent with the same. The amount of solvent utilized depends upon the scale of the reaction and specifically the amount of rapamycin starting material and nitrosobenzene present in the reaction mixture. One of skill in the art would readily be able to determine the amount of solvent required.

Typically, the solution containing the nitrosobenzene, rapamycin starting material, and solvent is maintained at elevated temperatures, and preferably a temperature that does not promote decomposition of the rapamycin and nitrosobenzene. In one embodiment, the solution is maintained a temperature of about 30 to about 70° C., and preferably about 50° C. The components are heated for a period of time sufficient to permit reaction between the rapamycin and nitrosobenzene. One of skill in the art using known techniques would readily be able to monitor the progress of the reaction during heating and thereby determine the amount of time required to perform the reaction. In one preferred embodiment, the rapamycin and nitrosobenzene are combined with p-dioxane and maintained at a temperature of about 50° C.

Isolation and purification of the rapamycin analogue is well within one of skill in the art and include chromatography including, without limitation, and recrystallization, high performance liquid chromatography (HPLC) such as reverse phase HPLC, and normal phase HPLC, and size-exclusion chromatography.

Once the rapamycin analogue is obtained, it can be reduced to form a more saturated rapamycin analogue. One of skill in the art would readily be able to select a suitable reducing agent for use in the present invention. Preferably, reduction of the rapamycin analogue can be effected using a hydrogenation agent. One of skill in the art would readily be able to select a suitable hydrogenation agent for use in the present invention. Typically, transition metal catalysts or transition metals on a support, preferably a carbon support, among others, in the presence hydrogen gas, are utilized to carry out the reduction. In a preferred embodiment, the reduction is performed using palladium metal on carbon in the presence of hydrogen gas.

Reduction of the rapamycin analogue is typically carried out in a solvent. A variety of solvents can be utilized in the reduction and include, without limitation, alcohols such as methanol. However, one of skill in the art would readily be able to select a suitable solvent for use in the present invention and depending on the hydrogenation catalyst and rapamycin analogue being reduced. The amount of solvent depends on the scale of the reaction, and specifically the amount of rapamycin analogue being reduced.

The amount of hydrogenation agent utilized in the present invention can readily be determined by one of skill in the art. However, one of skill in the art would be able to determine and adjust the amount of hydrogenation agent necessary to perform the reduction and to form the more saturated rapamycin analogues of the present invention. Further, a variety of apparatuses can be utilized to perform the hydrogenation of the present invention and include Parr apparatuses, among others. The selection of the particular apparatus for the hydrogenation is well within one of skill in the art.

A preferred method of preparing the rapamycin analogues of the present invention is summarized in Scheme 1 below:

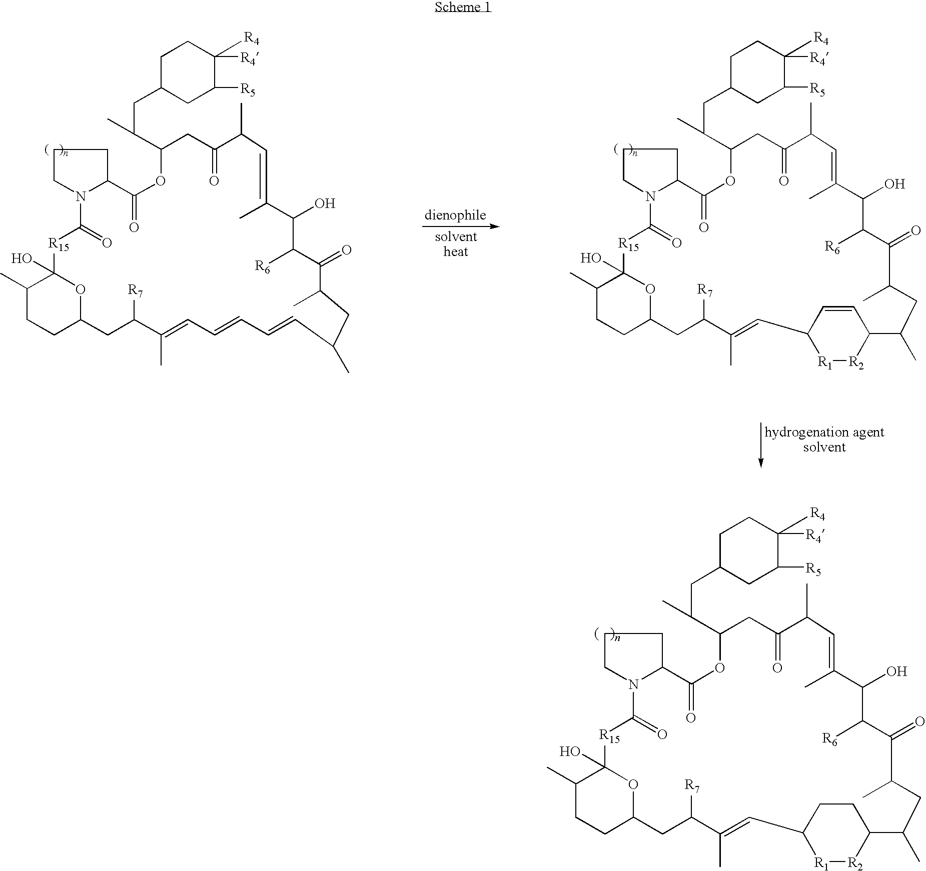

where R₁, R₂, R₄, R_{4′}, R₆, R₇, R₁₅, and n are defined above.

The rapamycin analogues can be utilized in the form of pharmaceutically acceptable salts, prodrugs, or metabolites thereof derived from pharmaceutically or physiologically acceptable acids or bases. These salts include, but are not limited to, the following salts with mineral or inorganic acids such as hydrochloric acid, sulfuric acid, nitric acid, phosphoric acid and organic acids such as acetic acid, oxalic acid, succinic acid, and maleic acid. Other salts include salts with alkali metals or alkaline earth metals, such as sodium, potassium, calcium or magnesium in the form of esters, carbamates and other conventional “pro-drug” forms, which, when administered in such form, convert to the active moiety in vivo.

Additional synthetic routes and characterization of the rapamycin analogues are provided in Examples 1-3 of commonly assigned co-pending published application US 2006/0135549, entitled “Rapamycin Analogues and the Uses Thereof in the Treatment of Neurological, Proliferative, and Inflammatory Disorders,” published on Jun. 22, 2006, referenced hereinabove.

Other examples of rapamycin analogues that can be used in the methods of the invention are disclosed in commonly owned published application U.S. 2006/0135550 entitled “Rapamycin Derivatives and the Uses Thereof in the Treatment of Neurological Disorders,” published on Jun. 22, 2006, from U.S. Ser. No. 11/300,941, the entire content of which is hereby incorporated by reference.

In other embodiments, the immunophilin ligand is a meridamycin analogue. Examples of meridamycin analogues that can be used in the methods of the invention include those disclosed in, e.g., U.S. 2005/0197379, U.S. 2005/0272133, U.S. 2005/0197356, WO 2005/084673, WO 2005/085257, as well as the following commonly owned provisional applications: U.S. Ser. No. 60/664,483 entitled “Meridamycin Derivatives and Uses Thereof,” filed Mar. 23, 2005 (publicly available through USPTO PAIR; and U.S. Ser. No. 60/779,940 entitled “Meridamycin Analogues for the Treatment of Neurodegenerative Disorders,” filed Mar. 7, 2006. (The entire contents of all of which are hereby incorporated by reference.) Some of the neurotrophic effects of the immunophilin ligands disclosed may be mediated by the formation of complexes described herein. In one embodiment, the meridamycin analogue has the chemical formula of compound I in U.S. 2005/0197379.

Several of the aforesaid rapamycin and meridamycin analogues have been demonstrated to have potent neurotrophic (e.g., neuroprotective, neuroregenerative and/or stimulating neurite outgrowth) activities in cultured cortical, dopaminergic and spinal cord neurons.

### Immunophilin Complexes

In one aspect, the invention relates to the discovery of, immunophilin complexes. In some embodiments, the complexes includes an immunophilin ligand (e.g., a rapamycin or a meridamycin analogue as described herein), an immunophilin (e.g., FKBP52) or a functional variant thereof, and a calcium channel subunit (e.g., a β1 subunit of the voltage gated L-type calcium channel) or a functional variant thereof.

As used herein, the terms “binding” and “complex formation” refer to a direct or indirect association between two or more molecules, e.g., polypeptides, macrolides, among others. Direct associations may include, for example, covalent, electrostatic, hydrophobic, ionic and/or hydrogen-bond interactions under physiological conditions. Indirect associations include, for example, two or more molecules that are part of a complex but do not have a direct interaction. In one embodiment, the association between the molecules is sufficient to maintain a stable complex under physiological conditions.

A complex of the invention may be obtained in isolated, recombinant, or purified form. The term “purified” or “isolated” as qualifiers of “protein” or “complex” refers to a preparation of a protein or proteins which are substantially free of other proteins normally associated with the protein (s) in a cell or cell lysate. For example, the phrase “substantially free” encompasses preparations comprising less than 40%, 30%, 20% (by dry weight) contaminating protein, and typically comprises less than 5% contaminating protein. By “purified” or “isolated,” it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” or “isolated” as used herein preferably means at least 80% by dry weight, typically in the range of 85% by weight, more typically 95-99% or higher by weight, of biological macromolecules of the same type present (but water, buffers, and other small molecules, especially molecules having a molecular weight of less than 5000, can be present). In one embodiment, the complex or protein is substantially free of purification materials, e.g., matrices or other materials. In other embodiments, the complex or protein is associated with the purification materials.

The term “recombinant” “protein” or “complex” refers to a protein(s) that form a complex, which are produced by recombinant DNA techniques. Generally, the DNA(s) encoding the expressed protein(s) is inserted into a suitable expression vector which is in turn used to transform a host cell (also referred to herein as a “recombinant cell”) to produce the heterologous protein. Moreover, the phrase “derived from,” with respect to a recombinant gene encoding the recombinant protein is meant to include within the meaning of “recombinant protein” those proteins having an amino acid sequence of a native protein, or an amino acid sequence similar thereto which is generated by mutations including substitutions, insertions, and deletions of a naturally occurring protein.

In an embodiment, the invention provides a complex prepared, for example, by extraction from a cell, e.g., an immunophilin-treated cell, that comprises the components of the complex (e.g., a naturally occurring or a recombinant cell). Extraction from a cell may be accomplished by any of the methods known in the art. For example, a complex may be extracted from the cell by a series of traditional protein purification steps, such as centrifugation, gel filtration, ion exchange chromatography, affinity chromatography and/or affinity purification. It will generally be preferable to select purification steps and conditions that do not dissociate the complex. As described in the appended Examples, a lysis buffer (e.g., 6 ml; 50 mM Tris, pH 7.4, 250 mM NaCl, 5 mM EDTA, 50 mM NaF, 1 mM Na₃VO₄, 1% Nonidet P40 (NP40), 0.1% mercaptoethanol and 2% protease inhibitor cocktails) can be used. For example, affinity matrices linking an immunophilin ligand, e.g., a rapamycin analog, to a resin can be prepared as described by Fretz et al. (1991) J. Am. Chem. Soc. 113:1409). In one embodiment, affinity matrices can be prepared by using Affi-gel10 resin through amino-phenyl-butyric acid (FIG. 1). Briefly, the amino group of amino-phenyl-butyric acid can be protected by treating with a protecting group such as diallyldicarbonate. The acid group of the resulting complex can be activated with PhOP(O)Cl₂ DMF complex in CH₂Cl₂. After the reaction is quenched, the ester product can be purified by, e.g., HPLC, and characterized by, e.g., MS and NMR. After removing the allyloxycarbonyl group, the amino group of the product can be linked to Affigel-10 matrix. The resulting Affigel-immunophilin ligand affinity matrix can be washed and stored. After extraction, aliquots of cell lysated can be mixed with affinity beads, such as Affigel10-immunophilin ligand. Beads can be analyzed on, e.g., 4-20% SDS-PAGE gel. The protein bands can be digested and further analyzed by, e.g., FT-ICR-MS analysis.

In other embodiments, the complex can be prepared by purifying recombinant polypeptides expressed in cells, such as E. coli, and reconstituting the complex in vitro. In certain embodiments, one or more of the constituent polypeptides of a complex is expressed from an endogenous gene of a cell. In certain embodiments, complexes are recombinant complexes wherein one or more of the constituent polypeptides are expressed from a recombinant nucleic acid. In certain embodiments, the invention also includes labeled protein complexes, wherein at least one polypeptide of the complex is labeled. For example, the label is a detectable label can be chosen from, e.g., one or more of radioisotopes, fluorescent compounds, enzymes, and enzyme co-factors. In another embodiment, the label facilitates purification, isolation, or detection of the polypeptide. The label may be a polyhistidine, FLAG, Glu-Glu, glutathione S transferase (GST), thioredoxin, protein A, protein G, and an immunoglobulin heavy chain constant region. In one embodiment, the labeled protein is FKBP52. In another embodiment, the labeled protein is a calcium channel subunit. The labeled complex or a component thereof can be purified by an appropriate affinity purification (e.g. as described above, or by contacting the complex with a nickel or copper resin in the case of a hexahistidine tag, contacting with a glutathione resin in the case of a GST tag).

In certain embodiment, a complex of the invention is in water-soluble form (a “soluble complex”). For example, a soluble complex may include soluble cytoplasmic portions of an immunophilin and/or a calcium channel subunit. In other embodiments, the complex may be less soluble in water or in membrane-associated form. For example, a complex comprising a protein having a transmembrane domain will generally be water insoluble. Insoluble complexes may be prepared, for example, as lipid micelles, detergent micelles or mixed micelles comprising lipids, detergents and/or other components. Insoluble complexes may also be prepared as membrane fractions from a cell. A membrane fraction may be a crude membrane fraction, wherein the membrane portion is simply separated from the soluble portion of a cell by, for example, centrifugation or filtration. A membrane fraction may be further purified by, for example, affinity purification directed to an affinity tag present in one or more of the proteins of a complex. Where a complex is present in a lipid bilayer, the lipid bilayer may, for example, be a vesicle (optionally inverted, i.e., with the normally extracellular face facing inwards towards the interior of the vesicle) or a planar bilayer.

Crystallized forms of the complex are also within the scope of the invention.

In one embodiment, the complex is cross-linked. Crosslinked complexes can be prepared using crosslinking reagents which are multifunctional or bifunctional agents. Such agents include the diamine group of compounds, such as, for example, hexamethylenediamine, diaminooctane, ethylenediamine, 4-(4-N-Maleimidophenyl)butyric acid hydrazide.HCl(MPBH), 4-(N-Maleimidomethyl)cyclohexane-1-carboxy-hydrazide.HCl (M₂ C₂H), and 3-(2-Pyridyldithio)propionyl hydrazide (PDPH) and other amine alkenes. Examples of such crosslinking agents are glutaraldehyde, succinaldehyde, octanedialdehyde and glyoxal. Additional multifunctional crosslinking agents include halo-triazines, e.g., cyanuric chloride; halo-pyrimidines, e.g., 2,4,6-trichloro/bromo-pyrimidine; anhydrides or halides of aliphatic or aromatic mono- or di-carboxylic acids, e.g., maleic anhydride, (meth)acryloyl chloride, chloroacetyl chloride; N-methylol compounds, e.g., N-methylol-chloro acetamide; di-isocyanates or di-isothiocyanates, e.g., phenylene-1,4-di-isocyanate and aziridines. Other crosslinking agents include epoxides, such as, for example, di-epoxides, tri-epoxides and tetra-epoxides. For a representative listing of other available crosslinking reagents see, for example, the Pierce Catalog and Handbook, Pierce Chemical Company, Rockford, Ill. (1997) and also S. S. Wong, Chemistry of Protein Conjugation and Cross-Linking, CRC Press, Boca Raton, Fla. (1991).

Alternatively, reversible crosslinkers can be used. Examples of reversible crosslinkers are described in T. W. Green, Protective Groups in Organic Synthesis, John Wiley & Sons (Eds.) (1981). Any variety of strategies used for reversible protecting groups can be incorporated into a crosslinker suitable for at least one crosslinking in producing carbohydrate crosslinked glycoprotein crystals capable of feversible, controlled solubilization. Various approaches are listed, in Waldmann's review of this subject, in Angewandte Chmie Intl. Ed. Engl., 35, p. 2056 (1996). Other types of reversible crosslinkers are disulfide bond-containing crosslinkers.

The invention further provides methods for modulating (e.g., increasing) the formation and/or stability of a complex described herein. The method includes: contacting an immunophilin, e.g., an FKBP52 (e.g., a human FKBP52) or a functional variant thereof; and a subunit of the voltage gated L-type calcium channel, e.g., a β1 subunit (e.g., a human β1 subunit), or a functional variant thereof, with an immunophilin ligand, e.g., a rapamycin or meridamycin analogue as described herein, under conditions that allow the formation of the complex to occur. The contacting step can occur in vitro, e.g., in a cell lysate or in a reconstituted system. Alternatively, the method can be performed on cells (e.g., neuronal or cardiovascular cells) present in a subject, e.g., a human or an animal subject (e.g., an in vivo animal model).

The subject method can also be used on cells in culture. For example, cells (e.g., purified or recombinant cells) can be cultured in vitro and the contacting step can be effected by adding the immunophilin ligand, e.g., the rapamycin or meridamycin analogue, to the culture medium. Typically, the cell is a mammalian cell, e.g., a human cell. In some embodiments, the cell is a neuronal or a cardiovascular cell. In some embodiments, the cell is a recombinant cell, e.g., a host cell. Such methods include (i) introducing into the cell one or more polynucleotides encoding the immunophilin and/or the calcium channel subunit; (ii) contacting said cell with an immunophilin ligand, e.g., a rapamycin or meridamycin analog as described herein; (iii) thereby forming a complex.

### Host Cells

In another aspect, the invention features host cells comprising one or more nucleic acids encoding one or more of the polypeptide constituents of the complex disclosed herein. In one embodiment, the host cells contain a first nucleic acid that includes a nucleotide sequence encoding an immunophilin, e.g., an FKBP52 (e.g., a mammalian FKBP52 as described herein) or a functional variant thereof; and/or a second nucleic acid that includes a nucleotide sequence encoding a subunit of the voltage gated L-type calcium channel, e.g., a β1 subunit (e.g., a mammalian β1 subunit as described herein), or a functional variant thereof. In one embodiment, the first nucleic acid comprises a nucleotide sequence encoding the amino acid sequence shown as FIG. 13A-13B (SEQ ID NOs:6-7), or a sequence substantially identical thereto. In other embodiments, the second nucleic acid comprises a nucleotide sequence encoding the amino acid sequence shown as FIG. 12A-12E (SEQ ID NO:1-5), or a sequence substantially identical thereto.

“Host cells,” “recombinant cells,” and “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “recombinant nucleic acid” includes any nucleic acid that includes at least two sequences which are not present together in nature. A recombinant nucleic acid may be generated in vitro, for example by using the methods of molecular biology, or in vivo, for example by insertion of a nucleic acid at a novel chromosomal location by homologous or non-homologous recombination.

In some embodiments, host cells may be used, for example, for purifying, making or studying a protein or protein complex. Optionally, host cells may be used, for example, for testing compounds in assay protocols such as those described below.

In certain embodiments, recombinant expression of polypeptides of a complex of the invention may be performed separately, and complexes formed therefrom. In another embodiment, recombinant expression of such polypeptides of a complex of the invention may be performed in the same cell, and complexes formed therefrom.

Suitable host cells for recombinant expression include bacteria such as E. coli., Clostridium sp., Pseudomonas sp., yeast, plant cells, insect cells (such as) and mammalian cells such as fibroblasts, lymphocytes, U937 cells (or other promonocytic cell lines) and Chinese hamster ovary cells (CHO cells).

For the purpose of host cell expression, the recombinant nucleic acid may be operably linked to one or more regulatory sequences in an expression construct. Regulatory nucleotide sequences will generally be appropriate for the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. In a preferred embodiment, the expression vector contains a selectable marker gene to allow the selection of transformed host cells. Selectable marker genes are well known in the art and will vary with the host cell used.

The expression vector may also include a fusion domain (typically provided by the expression vector) so that the recombinant polypeptide of the invention is expressed as a fusion polypeptide with said fusion domain. The main advantage of fusion domains are that they assist identification and/or purification of said fusion polypeptide and also enhance protein expression level and overall yield.

### Antibodies

In yet another aspect, the invention features an antibody, or antigen-binding fragment thereof that binds to the complexes disclosed herein. In certain embodiments, the antibodies increase the formation and/or stability of a complex disclosed herein. In other embodiments, the antibodies, or antigen-binding fragments thereof, decrease or inhibit the formation and/or stability of a complex disclosed herein. Exemplary antibody molecules include full immunoglobulin molecules, or portions thereof that contain, for example, the antigen binding site (including those portions of immunoglobulin molecules known in the art as F(ab), F(ab′), F(ab′)₂, humanized chimeric antibody, and F(v)). Polyclonal or monoclonal antibodies can be produced by methods known in the art. (Kohler and Milstein (1975) Nature 256, 495-497; Campbell “Monoclonal Antibody Technology, the Production and Characterization of Rodent and Human Hybridomas” in Burdon et al (eds.) (1985) “Laboratory Techniques in Biochemistry and Molecular Biology”, Vol. 13, Elsevier Science Publishers, Amsterdam); Harlow and Lane (eds) (1988) In “Antibodies A Laboratory Manual”, Cold Spring Harbor Press, Cold Spring Harbor, N.Y; the contents of all of which are hereby incorporated by reference.

Purified complexes of the invention, or the polypeptide components thereof, can be used to immunize animals to obtain polyclonal and monoclonal antibodies which specifically react with the complex. Such antibodies may be obtained using the entire complex or full length polypeptide components as an immunogen, or by using fragments thereof. Smaller fragments of the polypeptides may also be used to immunize animals. The peptide immunogens additionally may contain a cysteine residue at the carboxyl terminus and are conjugated to a hapten such as keyhole limpet hemocyanin (KLH). Additional peptide immunogens may be generated by replacing tyrosine residues with sulfated tyrosine residues. Methods for synthesizing such peptides are known in the art, as described in, for example, Ausbel et al. (eds) (1987) In Current Protocols In Molecular Biology, John Wiley and Sons (New York, N.Y.).

Modified antibodies, or antigen-binding fragments thereof, can be generated by techniques known in the art as disclosed in, e.g., Wood et al., International Publication WO 91/00906, Kucherlapati et al., International Publication WO 91/10741; Lonberg et al., International Publication WO 92/03918; Kay et al., International Publication WO 92/03917; Lonberg et al. (1994) Nature 368:856-59; Green et al. (1994) Nat. Genet. 7:13-21; Morrison et al. (1994) Proc. Natl. Acad. Sci. U.S.A. 81:6851-55; Bruggeman et al. (1993) Year Immunol. 7:33-40; Tuaillon et al. (1993) Proc. Natl. Acad. Sci. U.S.A. 90:3720-24; Bruggeman et al. (1991) Eur. J. Immunol. 21:1323-1326; Larrick et al. (1991) Biotechniques 11:152-56; Robinson et al., International Patent Application PCT/US86/02269; Akira et al., European Patent Application 184,187; Taniguchi, European Patent Application 171,496; Morrison et al., European Patent Application 173,494; Neuberger et al. International Publication WO 86/01533; Cabilly et al. U.S. Pat. No. 4,816,567; Better et al. (1988) Science 240:1041-43; Liu et al. (1987) Proc. Natl. Acad. Sci. U.S.A. 84:3439-43; Liu et al. (1987) J. Immunol. 139:3521-26; Sun et al. (1987) Proc. Natl. Acad. Sci. U.S.A. 84:214-18; Nishimura et al. (1987) Canc. Res. 47:999-1005; Wood et al. (1985) Nature 314:446-49; Shaw et al. (1988) J. Natl. Cancer Inst. 80:1553-59; Morrison (1985) Science 229:1202-07; Oi et al. (1986) BioTechniques 4:214; and Queen et al. U.S. Pat. Nos. 5,585,089, 5,693,761 and 5,693,762, the contents of all of which are hereby incorporated by reference. Those methods include isolating, manipulating, and expressing the nucleic acid sequences that encode all or part of immunoglobulin Fv variable regions from at least one of a heavy or light chain. Sources of such nucleic acids are known to those skilled in the art and, for example, may be obtained from a hybridoma producing an antibody against a predetermined target. The recombinant DNA encoding the recombinant antibody, or fragment thereof, can then be cloned into an appropriate expression vector.

Assays for Identifying Test Compounds that Modulate Formation of the Complex

In another aspect, the invention provides a method, or an assay, for identifying a test compound that modulates, e.g., inhibits or increases, the formation and/or stability of a complex that includes the test compound, an immunophilin, and a calcium channel subunit. The method, or the assay, includes: contacting a sample that includes an immunophilin or a functional variant thereof, and β subunit or a functional variant thereof with a test compound under conditions that allow the formation of the complex; detecting the presence of the complex in the sample contacted with the test compound relative to a reference sample (e.g., a control sample not exposed to the test agent, or a control sample exposed to rapamycin). A change (e.g., an increase or a decrease) in the level of the complex in the presence of the test compound, relative to the level of the complex in the reference sample, indicates that said test compound affects (e.g., increases or decreases) the formation and/or stability of said complex. Test compounds that increase complex formation by, e.g., about 1.5, 2, 5, 10 fold or higher, relative to a reference sample are preferred.

Test compounds can be obtained, for example, from bacteria, actinomycetes (e.g., S. hygroscopicus), yeast or other organisms (e.g., natural products), produced chemically (e.g., small molecules, including peptidomimetics), or produced recombinantly. For example, polyketides can be produced from naturally occurring or genetically modified Streptomyces species, as for example, described in U.S. 2005/0272133, U.S. 2005/0197379. Modified forms of the rapamycin and meridamycin analogues disclosed herein can be alternatively by chemical synthesis.

The complex of the invention allows for the generation of new modified macrolides, e.g., modified forms of the rapamycin and meridamycin analogues disclosed herein. The purified complex can be used for determination of a three-dimensional crystal structure, which can be used for modeling intermolecular interactions. For example, crystal structures of the complex can be determined and modifications of the structure can be generated by performing rational drug design using techniques known in the art. Numerous computer programs are available for rational drug design, computer modeling, model building as described in U.S. 2005/0288489A1, the contents of which are incorporated by reference herein.

A variety of assay formats will suffice and, in light of the present disclosure, those not expressly described herein will nevertheless be comprehended by one of ordinary skill in the art. Assay formats which approximate such conditions as formation of protein complexes, enzymatic activity, and may be generated in many different forms, and include assays based on cell-free systems, e.g., purified proteins or cell lysates, as well as cell-based assays which utilize intact cells. Simple binding assays can be used to detect compounds that inhibit or potentiate the interaction between components of the complex, or the binding of the complex to a substrate.

In certain embodiments, the present invention provides reconstituted protein preparations including a polypeptide of the complex, and one or more interacting polypeptides of the complex. In one embodiments, all components or the complex are added simultaneously in a reaction mixture. In other embodiments, the reaction mixture is prepared by adding the components sequentially, e.g., forming a mixture of the immunophilin and the calcium channel, and adding the immunophilin ligand. Alternatively, the immunophilin ligand can be added to the immunophilin or the calcium channel. Any order or combination of the components can be used. Assays of the present invention include labeled in vitro protein-protein binding assays, immunoassays for protein binding, and the like. In one embodiment, the sample is a cell lysate or a reconstituted system. The reconstituted complex can comprise a reconstituted mixture of at least semi-purified proteins. By semi-purified, it is meant that the proteins utilized in the reconstituted mixture have been previously separated from other cellular proteins. For instance, in contrast to cell lysates, proteins involved in the complex formation are present in the mixture to at least 50% purity relative to all other proteins in the mixture, and more preferably are present at 90-95% purity. In certain embodiments, the reconstituted protein mixture is derived by mixing highly purified proteins such that the reconstituted mixture substantially lacks other proteins (such as of cellular origin) which might interfere with or otherwise alter the ability to measure the complex assembly and/or disassembly. In certain embodiments, assaying in the presence and absence of a candidate compound, can be accomplished in any vessel suitable for containing the reactants. Examples include microtitre plates, test tubes, and micro-centrifuge tubes.

In certain embodiments, drug screening assays can be generated which detect test compounds on the basis of their ability to interfere with assembly, stability, or function of a complex of the invention. Detection and quantification of the complex provide a means for determining the compound's efficacy at inhibiting (or potentiating) interaction between the components. The efficacy of the compound can be assessed by generating dose response curves from data obtained using various concentrations of the test compound. Moreover, a control assay can also be performed to provide a baseline for comparison. In the control assay, the formation of complexes is quantitated in the absence of the test compound.

In certain embodiments, association between any two polypeptides in a complex or between the complex and a substrate polypeptide, may be detected by a variety of techniques, many of which are effectively described above. For instance, modulation in the formation of complexes can be quantitated using, for example, detectably labeled proteins (e.g., radiolabeled, fluorescently labeled, or enzymatically labeled), by immunoassay, or by chromatographic detection. Surface plasmon resonance systems, such as those available from Biacore International AB (Uppsala, Sweden), may also be used to detect protein-protein interaction.

In certain embodiments, one of the polypeptides of a complex can be immobilized to facilitate separation of the complex from uncomplexed forms of one of the polypeptides, as well as to accommodate automation of the assay. Affinity matrices or beads are described herein that contain the immunophilin ligand (or other components of the complex) that permits other components of the complex to be bound to an insoluble matrix. Test compound are incubated under conditions conducive to complex formation. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes can be dissociated from the matrix, separated by SDS-PAGE gel, and the level of interacting polypeptide found in the matrix-bound fraction quantitated from the gel using standard electrophoretic techniques.

Alternatively, the assays can be performed using cells in culture, e.g., purified cultured or recombinant cells. For example, a two-hybrid assay (also referred to as an interaction trap assay) can be used for detecting the interaction of any two polypeptides in the complex, and for subsequently detecting test compounds which inhibit or potentiate binding of the proteins to one and other (see also, U.S. Pat. No. 5,283,317; WO94/10300; Zervos et al. (1993) Cell 72: 223-232; Madura et al. (1993) J. Biol. Chem. 268: 12046-12054; Bartel et al. (1993) Biotechniques 14: 920-924; and Iwabuchi et al. (1993) Oncogene 8: 1693-1696), the contents of all of which are incorporated by reference.

In many drug screening programs which test libraries of compounds and natural extracts, high throughput assays are desirable in order to maximize the number of compounds surveyed in a given period of time. Assays of the present invention which are performed in cell-free systems, such as may be developed with purified or semi-purified proteins or with lysates, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with other proteins or changes in enzymatic properties of the molecular target.

In certain embodiments, activities of a protein complex may include, without limitation, a protein complex formation, which may be assessed by immunoprecipitation and analysis of co-immunoprecipitated proteins or affinity purification and analysis of co-purified proteins. Fluorescence Resonance Energy Transfer (FRET)-based assays may also be used to determine complex formation. Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. FRET-based assays are described in U.S. Pat. No. 5,981,200, the contents of which are incorporated by reference.

In general, where a screening assay is a binding assay (whether protein-protein binding, compound-protein binding, etc.), one or more of the molecules may be joined to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g., albumin, detergents, etc that are used to facilitate optimal protein-protein binding and/or reduce nonspecific or background interactions. Reagents that improve the efficiency of the assay, such as protease inhibitors, nuclease inhibitors, anti-microbial compounds, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening.

In certain embodiments, the test compounds can be further assayed to identify compounds that modulate calcium channel activity. For example, the effect of a test compound can be measured by testing calcium channel activity of a eukaryotic cell having a functional calcium channel (e.g., a heterologous channel) when such cell is exposed to a solution containing the test compound and a calcium channel selective ion, and comparing the measured calcium channel activity to the calcium channel activity of the same cell or a substantially identical control cell in a solution not containing the test compound. The cell is maintained, in one embodiment, in a solution having a concentration of calcium channel selective ions sufficient to provide an inward current when the channels open. Methods for practicing such assays are known to those of skill in the art. For example, for similar methods applied with Xenopus laevis oocytes and acetylcholine receptors, see, Mishina et al. (1985) Nature 313:364; Noda et al. (1986) Nature 322:826-828; Claudio et al. (1987) Science 238:1688-1694.

The assays are based on cells that express functional calcium channels and measure functionally, such as electrophysiologically, the ability of a test compound to potentiate, antagonize or otherwise modulate the magnitude and duration of the flow of calcium channel selective ions, such as Ca⁺⁺ or Ba⁺⁺, through the heterologous functional channel. The amount of current, which flows though the recombinant calcium channels of a cell may be determined, in one embodiment, directly, such as electrophysiologically, or, in another embodiment, by monitoring an independent reaction which occurs intracellularly and which is directly influenced in a calcium (or other) ion dependent manner.

Any method for assessing the activity of a calcium channel may be used in conjunction with the methods described herein. For example, in one embodiment of the method for testing a compound for its ability to modulate calcium channel activity, the amount of current is measured by its modulation of a reaction which is sensitive to calcium channel selective ions and uses a eukaryotic cell which expresses a heterologous calcium channel and also contains a transcriptional control element operatively linked for expression to a structural gene that encodes an indicator protein. The transcriptional control element used for transcription of the indicator gene is responsive in the cell to a calcium channel selective on, such as Ca²⁺ and Ba⁺. The details of such transcriptional based assays are described, for example, in PCT International Patent Application No. PCT/US91/5625.

In other embodiments, electrophysiological methods for measuring calcium channel activity, which are known to those of skill in the art and exemplified herein may be utilized for the indicated purposes. Any such methods may be used in order to detect the formation of functional calcium channels and to characterize the kinetics and other characteristics of the resulting currents. Pharmacological studies may be combined with the electrophysiological measurements, in other embodiments, in order to further characterize the calcium channels.

In general, activity of a given test compound in the nervous system can be assayed by detecting the compound's ability to affect one of more of: promote neurite outgrowth, protect neurons from damage by chemical treatments, promote the growth of neurons or neuronal cells, recover lost or damaged motor, functional or cognitive ability associated with nervous tissue or organs of the nervous system, or regenerate neurons. For example, isolated neuronal cell cultures (e.g., dopaminergic, cortical, DRG cell cultures) can be isolated and cultured by methods known in the art (see e.g., Pong et al. (1997) J. Neurochem. 69:986-994; Pong et al. (2001) Exp Neurol. 171(1):84-97). Changes in neuronal activity, differentiation, survival can be detected and quantified using art recognized techniques as described in, e.g., US 2005/0197356 (describing examples showing measuring changes in 3H-dopamine uptake and neurofilament content in cultured dopaminergic neurons and cortical neurons, respectively). Alternatively, neuronal activities can be characterized in cultured neural cell lines, e.g., neuroblastoma cell lines, pheochromocytoma cells (PC12 cells), F11. Activities in vitro can be useful in identifying agents that can be used to treat and/or ameliorate a number of human neurodegenerative conditions, including but not limited to, Parkinson's disease; Alzheimer's disease; amyotrophic lateral sclerosis (ALS); traumatic injury; spinal cord injury; multiple sclerosis; diabetic neuropathy; neuropathy associated with medical treatments such as chemotherapy; ischemia or ischemia-induced injury; stroke, among others.

Methods for detecting neuronal activity include, for example, neuroprotective assays where a compound is tested for its ability to protect against glutamate neurotoxicity. Sensory neuronal cultures (DRG) can also be assayed for neurite outgrowth, and assayed for neurotrophic activity. Cultured cells are treated with an immunophilin ligand and later assayed for the presence of new neurite fibers. Immunohistochemistry can aid in the visualization and quantitation of neurites as compared to control.

A number of animal models and cell culture assays have been developed and can be relied on for their clinical relevance to disease treatments, including the human diseases noted above. Each of the following references can be used as a source for these assays, and all of them are specifically incorporated herein by reference in their entirety for that purpose: Steiner, et al., Proc. Natl. Acad. Sci. U.S.A. 94: 2019-2024 (1997); Hamilton, et al., Bioorgan. Med. Chem. Lett. 7:1785-1790 (1997); McMahon, et al., Curr. Opin. Neurobiol. 5:616-624 (1995); Gash, et al., Nature 380:252-255 (1996); Gerlach, et al., Eur. J. Pharmacol.-Mol. Pharmacol. 208:273-286 (1991); Apfel, et al., Brain Res. 634:7-12 (1994); Wang, et al., J. Pharmacol. Exp. Therap. 282:1084-1093 (1997); Gold, et al., Exp. Neurol. 147:269-278 (1997); Hoffer et al., J. Neural Transm. [Suppl.]49:1-10 (1997); and Lyons, et al., PNAS 91:3191-3195 (1994).

### Therapeutic and Prophylactic Uses

In yet another aspect, the invention provides methods for modulating a function (e.g., calcium channel activity (e.g., voltage-gated calcium channel activity), in a cell (e.g., a mammalian cell) that expresses an immunophilin, e.g., an FKBP52 or a functional variant thereof and a subunit of the voltage gated L-type calcium channel, e.g., a β1 subunit, or a functional variant thereof. In one embodiment, the calcium channel or FKBP52 activity or expression is inhibited. In those embodiments where calcium channel activity is inhibited, neurite outgrowth and/or survival is preferably stimulated. Typically, the cell used in the methods of the invention is a mammalian cell, e.g., a human cell (e.g., a neuronal or a cardiovascular cell). In some embodiments, the methods include contacting the cell with an immunophilin ligand, e.g., a rapamycin or a meridamycin analogue as described herein, under conditions that allow the formation of a complex described herein to occur, thereby inhibiting the calcium channel activity.

In related embodiments, the methods include contact the cell (e.g., a dopaminergic, cholinergic, cortical, and spinal cord neuronal cell) with an antagonist of a calcium channel β subunit, e.g., a β1 subunit of the voltage gated L-type calcium channel. The antagonist can also be an inhibitor of activity and/or expression of the calcium channel β subunit. The term “antagonist” as used herein refers to an agent which reduces, inhibits or otherwise diminishes one or more biological activities of a calcium channel β subunit (e.g., β1 subunit). Antagonism does not necessarily indicate a total elimination of the calcium channel β subunit biological activity. In one embodiment, the antagonist is an immunophilin ligand, e.g., a rapamycin or meridamycin analogue as described herein. Typically, the immunophilin ligand is administered in an amount sufficient to form and/or stabilize a complex that includes the ligand, an immunophilin or a functional variant thereof, and a calcium channel subunit or a functional variant thereof. In other embodiment, the antagonist is an inhibitor of transcription of the calcium channel β subunit, e.g., a nucleic acid inhibitor (e.g., RNAi) as described in more detail herein.

The methods of the invention can be performed in cells in cultured medium. Alternatively, the method can be performed on cells (e.g., neuronal or cardiovascular cells) present in a subject, e.g., as part of an in vivo (e.g., therapeutic or prophylactic) protocol, or in an animal subject (e.g., an in vivo animal model).

Accordingly, methods of treating or preventing, in a subject, a disorder associated with calcium channel dysfunction, are encompassed by the present invention. The method includes administering to a subject an immunophilin ligand, e.g., a rapamycin or meridamycin analogue, in an amount sufficient to form and/or stabilize a complex that includes the ligand, an immunophilin or a functional variant thereof, and a calcium channel subunit or a functional variant thereof, thereby treating or preventing the disorder. The method can, optionally, include the step(s) of identifying (e.g., evaluating, diagnosing, screening, and/or selecting) a subject at risk of having, or having, one or more symptoms associated with a disorder involving calcium channel dysfunction. The subject can be a mammal, e.g., a human suffering from, e.g., a neurodegenerative or a cardiovascular disorder. For example, the subject is a human (e.g., a human patient) suffering from a disorder chosen from one or more of stroke, Parkinson's disease, migraine, cerebellar ataxia, angina, epilepsy, hypertension, ischemia, or cardiac arrhythmias.

As used herein, the term “subject” is intended to include human and non-human animals. Preferred human animals include a human patient having a disorder characterized by abnormal calcium channel activity. The term “non-human animals” includes vertebrates, e.g., mammals and non-mammals, such as non-human primates, rodents, sheep, dog, cow, chickens, amphibians, reptiles, etc. The subject can be, for example, a mammal, e.g., a human suffering from, e.g., a neurodegenerative or a cardiovascular disorder.

The phrase “therapeutically effective amount” of an immunophilin ligand refers to an amount of an agent which is effective, upon single or multiple dose administration to a subject, e.g., a human patient, at treating the subject. The term “treating” or “treatment” includes curing, reducing the severity of, ameliorating one or more symptoms of a disorder, or in prolonging the survival of the subject beyond that expected in the absence of such treatment. Similarly, the phrase “a prophylactically effective amount” of an immunophilin ligand refers to an amount of an agent which is effective, upon single- or multiple-dose administration to a subject, e.g., a human patient, in preventing or delaying the occurrence of the onset or recurrence of a disorder, e.g., a disorder as described herein.

The immunophilin ligand, e.g., the rapamycin analogue, can be administered alone, or in combination with one or more agents, e.g., therapeutic agents. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment. In one embodiment, the second agent is a calcium channel antagonist, e.g., an antagonists of an L-type calcium channel. Examples of antagonists of L-type calcium channels include dihydropyridines; phenylalkylamines (e.g., verapamil, gallpamil, and thiapamil); benzothiazepines; diphenylbutylpiperidine class of antischizophrenic neuroleptic drugs (e.g., pimozide, fluspiridine, penfluridol and clopimozide); as well as nifedipine, carbamazepine, diltiazem, nicardipine, nimodipine, and nitredipine.

Exemplary disorders associated with calcium channel dysfunction include stroke; Parkinson's disease; migraine (e.g., congenital migraine); cerebellar ataxia; angina; epilepsy; hypertension; ischemia (e.g., cardiac ischemia); cardiac arrhythmias; stroke; head trauma or spinal injury, or other injuries to the brain, peripheral nervous, central nervous, or neuromuscular system; chronic, neuropathic and acute pain; mood disorders; schizophrenia; depression; anxiety; psychoses; drug addiction; alcohol dependence and urinary incontinence.

Examples of other conditions associated with dysfunction of calcium (Ca²⁺) ion channels, include, but not limited to, malignant hyperthermia, central core disease, cathecolaminergic polymorphic ventricular tachycardia, and arrhythmogenic right ventricular dysplasia type 2 (ARVD-2). Examples of neurological disorders that can be treated using the methods of the invention include Alzheimer's disease; Huntington's disease; spinal cord injury; traumatic brain injury; Lewy body dementia; Pick's disease; Niewmann-Pick disease; amyloid angiopathy; cerebral amyloid angiopathy; systemic amyloidosis; hereditary cerebral hemorrhage with amyloidosis of the Dutch type; inclusion body myositis; mild cognitive impairment; Down's syndrome; and neuromuscular disorders, including amyotrophic lateral sclerosis (ALS), multiple sclerosis, and muscular dystrophies including Duchenne dystrophy, Becker muscular dystrophy, Facioscapulohumeral (Landouzy-Dejerine) muscular dystrophy, and limb-girdle muscular dystrophy (LGMD). The immunophilin ligands are also useful as neuroprotective and/or neuroregenerative agents, e.g., in restoring some neurological and/or neuromuscular or other function following onset of one of the above conditions and/or injury, stroke, or other trauma.

Examples of additional cardiovascular disorders that can be treated include, but not limited to, congestive heart failure; arrhythmogenic syndromes, including paroxysomal tachycardia, delayed after depolarizations, ventricular tachycardia, sudden tachycardia, exercise-induced arrhythmias, long QT syndromes, and bidirectional tachycardia; thromboembolic disorders, including arterial cardiovascular thromboembolic disorders, venous cardiovascular thromboembolic disorders, and thromboembolic disorders in the chambers of the heart; atherosclerosis; restenosis; peripheral arterial disease; coronary bypass grafting surgery; carotid artery disease; arteritis; myocarditis; cardiovascular inflammation; vascular inflammation; coronary heart disease (CHD); unstable angina (UA); unstable refractory angina; stable angina (SA); chronic stable angina; acute coronary syndrome (ACS); first or recurrent myocardial infarction; acute myocardial infarction (AMI); myocardial infarction; non-Q wave myocardial infarction; non-STE myocardial infarction; coronary artery disease; ischemic heart disease; ischemic sudden death; transient ischemic attack; stroke; peripheral occlusive arterial disease; venous thrombosis; deep vein thrombosis; thrombophlebitis; arterial embolism; coronary arterial thrombosis; cerebral arterial thrombosis; cerebral embolism; kidney embolism; pulmonary embolism; thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis; thrombosis resulting from atherosclerosis, surgery or surgical complications, prolonged immobilization, arterial fibrillation, congenital thrombophilia, cancer, diabetes, effects of medications or hormones, and complications of pregnancy; cardiac arrhytmias including supraventricular arrhythmias, atrial arrhythmias, atrial flutter, atrial fibrillation; other diseases listed in Heart Disease: A Textbook of Cardiovascular Medicine, 2 Volume Set, 6th Edition, 2001, Eugene Braunwald, Douglas P. Zipes, Peter Libby, Douglas D. Zipes; and in the preparation of medicaments therefor.

In a further embodiment, the cardiovascular disease is chosen from one or more of: atherosclerosis; coronary heart disease (CHD); restensosis; peripheral arterial disease; coronary bypass grafting surgery; carotid artery disease; arteritis; myocarditis; cardiovascular inflammation; vascular inflammation; unstable angina (UA); unstable refractory angina; stable angina (SA); chronic stable angina; acute coronary syndrome (ACS); myocardial infarction; or acute myocardial infarction (AMI), including first or recurrent myocardial infarction, non-Q wave myocardial infarction, non-ST-segment elevation myocardial infarction and ST-segment elevation myocardial infarction.

The amount or dosage requirements of the immunophilin ligands can vary depending on the condition, severity of the symptoms presented and the particular subject being treated. One of skill in the art would readily be able to determine the amount of the immunophilin ligand required following the methods described herein. Preferably, the dosage of the immunophilin ligand is such that it is sufficient to form and/or stabilize a complex that includes the ligand, an immunophilin or a functional variant thereof, and a calcium channel subunit or a functional variant thereof. In some embodiments, the dosage can be tested in vitro following the teachings of the invention. In one embodiment, about 0.5 to 200 mg, about 0.5 to 100 mg, about 0.5 to about 75 mg is administered. In yet a further embodiment, about 1 to about 25 mg is administered. In another embodiment, about 0.5 to about 10 mg is administered, particularly when used in combination with another agent. In yet a further embodiment, about 2 to about 5 mg is administered. In yet another embodiment, about 5 to about 15 mg is administered.

Treatment can be initiated with dosages of the immunophilin ligand lower than those required to produce a desired effect and generally less than the optimum dose of the ligand. Thereafter, the dosage can be increased until the optimum effect under the circumstances is reached. Precise dosages will be determined by the administering physician based on experience with the individual subject being treated. In general, the compositions are most desirably administered at a concentration that will generally afford effective results without causing any harmful or deleterious side effects.

In certain embodiments, nucleic acid antagonists are used to decrease expression of an endogenous gene encoding the calcium channel β subunit (e.g., the (β1 subunit). In one embodiment, the nucleic acid antagonist is an siRNA that targets mRNA encoding the calcium channel β subunit. Other types of antagonistic nucleic acids can also be used, e.g., a dsRNA, a ribozyme, a triple-helix former, or an antisense nucleic acid. In some embodiments, nucleic acid antagonists can be directed to downstream effector targets of the calcium channel β subunit.

siRNAs are small double stranded RNAs (dsRNAs) that optionally include overhangs. For example, the duplex region of an siRNA is about 18 to 25 nucleotides in length, e.g., about 19, 20, 21, 22, 23, or 24 nucleotides in length. Typically, the siRNA sequences are exactly complementary to the target mRNA. dsRNAs and siRNAs in particular can be used to silence gene expression in mammalian cells (e.g., human cells). siRNAs also include short hairpin RNAs (shRNAs) with 29-base-pair stems and 2-nucleotide 3′ overhangs. See, e.g., Clemens et al. (2000) Proc. Natl. Acad. Sci. USA 97:6499-6503; Billy et al. (2001) Proc. Natl. Sci. USA 98:14428-14433; Elbashir et al. (2001) Nature. 411:494-8; Yang et al. (2002) Proc. Natl. Acad. Sci. USA 99:9942-9947; Siolas et al. (2005), Nat. Biotechnol. 23(2):227-31; 20040086884; U.S. 20030166282; 20030143204; 20040038278; and 20030224432.

Anti-sense agents can include, for example, from about 8 to about 80 nucleobases (i.e. from about 8 to about 80 nucleotides), e.g., about 8 to about 50 nucleobases, or about 12 to about 30 nucleobases. Anti-sense compounds include ribozymes, external guide sequence (EGS) oligonucleotides (oligozymes), and other short catalytic RNAs or catalytic oligonucleotides which hybridize to the target nucleic acid and modulate its expression. Anti-sense compounds can include a stretch of at least eight consecutive nucleobases that are complementary to a sequence in the target gene. An oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

Hybridization of antisense oligonucleotides with mRNA (e.g., an mRNA encoding the calcium channel β subunit) can interfere with one or more of the normal functions of mRNA. The functions of mRNA to be interfered with include all key functions such as, for example, translocation of the RNA to the site of protein translation, translation of protein from the RNA, splicing of the RNA to yield one or more mRNA species, and catalytic activity which may be engaged in by the RNA. Binding of specific protein(s) to the RNA may also be interfered with by antisense oligonucleotide hybridization to the RNA.

Exemplary antisense compounds include DNA or RNA sequences that specifically hybridize to the target nucleic acid, e.g., the mRNA encoding the calcium channel β subunit. The complementary region can extend for between about 8 to about 80 nucleobases. The compounds can include one or more modified nucleobases. Modified nucleobases may include, e.g., 5-substituted pyrimidines such as 5-iodouracil, 5-iodocytosine, and C5-propynyl pyrimidines such as C5-propynylcytosine and C5-propynyluracil. Other suitable modified nucleobases include N⁴—(C₁-C₁₂) alkylaminocytosines and N⁴, N⁴—(C₁-C₁₂) dialkylaminocytosines. Modified nucleobases may also include 7-substituted-8-aza-7-deazapurines and 7-substituted-7-deazapurines such as, for example, 7-iodo-7-deazapurines, 7-cyano-7-deazapurines, 7-aminocarbonyl-7-deazapurines. Examples of these include 6-amino-7-iodo-7-deazapurines, 6-amino-7-cyano-7-deazapurines, 6-amino-7-aminocarbonyl-7-deazapurines, 2-amino-6-hydroxy-7-iodo-7-deazapurines, 2-amino-6-hydroxy-7-cyano-7-deazapurines, and 2-amino-6-hydroxy-7-aminocarbonyl-7-deazapurines. Furthermore, N⁶—(C₁-C₁₂) alkylaminopurines and N⁶, N⁶—(C₁-C₁₂) dialkylaminopurines, including N⁶-methylaminoadenine and N⁶, N⁶-dimethylaminoadenine, are also suitable modified nucleobases. Similarly, other 6-substituted purines including, for example, 6-thioguanine may constitute appropriate modified nucleobases. Other suitable nucleobases include 2-thiouracil, 8-bromoadenine, 8-bromoguanine, 2-fluoroadenine, and 2-fluoroguanine. Derivatives of any of the aforementioned modified nucleobases are also appropriate. Substituents of any of the preceding compounds may include C₁-C₃₀ alkyl, C₂-C₃₀ alkenyl, C₂-C₃₀ alkynyl, aryl, aralkyl, heteroaryl, halo, amino, amido, nitro, thio, sulfonyl, carboxyl, alkoxy, alkylcarbonyl, alkoxycarbonyl, and the like.

Descriptions of other types of nucleic acid agents are also available. See, e.g., U.S. Pat. Nos. 4,987,071; 5,116,742; and 5,093,246; Woolf et al. (1992) Proc Natl Acad Sci USA; Antisense RNA and DNA, D. A. Melton, Ed., Cold Spring Harbor Laboratory, Cold Spring Harbor, N.Y. (1988); 89:7305-9; Haselhoff and Gerlach (1988) Nature 334:585-59; Helene, C. (1991) Anticancer Drug Des. 6:569-84; Helene (1992) Ann. N.Y. Acad. Sci. 660:27-36; and Maher (1992) Bioassays 14:807-15.

### Pharmaceutical Compositions

In one aspect, the present invention includes methods of preparing a pharmaceutical composition containing one or more immunophilin ligands. In other embodiments, pharmaceutical compositions containing the complexes described herein are disclosed. As used herein, compositions containing “an immunophilin ligand” or “the immunophilin ligand” are intended to encompass compositions containing one or more immunophilin ligands. The composition can be administered to a mammalian subject by several different routes and is desirably administered orally in solid or liquid form.

Solid forms, including tablets, capsules, and caplets, containing the immunophilin ligand can be formed by blending the immunophilin ligand with one or more of the components described above. In one embodiment, the components of the composition are dry or wet blended. In another embodiment, the components are dry granulated. In a further embodiment, the components are suspended or dissolved in a liquid and added to a form suitable for administration to a mammalian subject.

Liquid forms containing the immunophilin ligand can be formed by dissolving or suspending the immunophilin ligand in a liquid suitable for administration to a mammalian subject.

The compositions described herein containing the immunophilin ligand can be formulated in any form suitable for the desired route of delivery using a pharmaceutically effective amount of the immunophilin ligand. For example, the compositions of the invention can be delivered by a route such as oral, dermal, transdermal, intrabronchial, intranasal, intravenous, intramuscular, subcutaneous, parenteral, intraperitoneal, intranasal, vaginal, rectal, sublingual, intracranial, epidural, intratracheal, or by sustained release. Preferably, delivery is oral.

The oral dosage tablet composition of this invention can also be used to make oral dosage tablets containing derivatives of the immunophilin ligand, including, but not limited to, esters, carbamates, sulfates, ethers, oximes, carbonates, and the like which are known to those of skill in the art.

A pharmaceutically effective amount of the immunophilin ligand can vary depending on the specific compound(s), mode of delivery, severity of the condition being treated, and any other active ingredients used in the composition. The dosing regimen can also be adjusted to provide the optimal therapeutic response. Several divided doses can be delivered daily, e.g., in divided doses 2 to 4 times a day, or a single dose can be delivered. The dose can however be proportionally reduced or increased as indicated by the exigencies of the therapeutic situation. In one embodiment, the delivery is on a daily, weekly, or monthly basis. In another embodiment, the delivery is on a daily delivery. However, daily dosages can be lowered or raised based on the periodic delivery.

The immunophilin ligands can be combined with one or more pharmaceutically acceptable carriers or excipients including, without limitation, solid and liquid carriers which are compatible with the compositions of the present invention. Such carriers include adjuvants, syrups, elixirs, diluents, binders, lubricants, surfactants, granulating agents, disintegrating agents, emollients, metal chelators, pH adjustors, surfactants, fillers, disintegrants, and combinations thereof, among others. In one embodiment, the immunophilin ligand is combined with metal chelators, pH adjustors, surfactants, fillers, disintegrants, lubricants, and binders. Adjuvants can include, without limitation, flavoring agents, coloring agents, preservatives, and supplemental antioxidants, which can include vitamin E, ascorbic acid, butylated hydroxytoluene (BHT) and butylated hydroxyanisole (BHA).

Binders can include, without limitation, cellulose, methylcellulose, hydroxymethylcellulose, carboxymethylcellulose calcium, carboxymethylcellulose sodium, hydroxypropylcellulose, hydroxypropylmethylcellulose phthalate, microcrystalline cellulose, noncrystalline cellulose, polypropylpyrrolidone, polyvinylpyrrolidone (povidone, PVP), gelatin, gum arabic and acacia, polyethylene glycols, starch, sugars such as sucrose, kaolin, dextrose, and lactose, cholesterol, tragacanth, stearic acid, gelatin, casein, lecithin (phosphatides), cetostearyl alcohol, cetyl alcohol, cetyl esters wax, dextrates, dextrin, glyceryl monooleate, glyceryl monostearate, glyceryl palmitostearate, polyoxyethylene alkyl ethers, polyoxyethylene castor oil derivatives, polyoxyethylene stearates, polyvinyl alcohol, and gelatin, among others. In one embodiment, the binder is povidone, hydroxypropylmethylcellulose, carboxymethylcellulose, or gelatin. In another embodiment, the binder is povidone.

Lubricants can include magnesium stearate, light anhydrous silicic acid, talc, stearic acid, sodium lauryl sulfate, and sodium stearyl furamate, among others. In one embodiment, the lubricant is magnesium stearate, stearic acid, or sodium stearyl furamate. In another embodiment, the lubricant is magnesium stearate.

Granulating agents can include, without limitation, silicon dioxide, microcrystalline cellulose, starch, calcium carbonate, pectin, crospovidone, and polyplasdone, among others.

Disintegrating agents or disintegrants can include croscarmellose sodium, starch, carboxymethylcellulose, substituted hydroxypropylcellulose, sodium bicarbonate, calcium phosphate, calcium citrate, sodium starch glycolate, pregelatinized starch or crospovidone, among others. In one embodiment, the disintegrant is croscarmellose sodium.

Emollients can include, without limitation, stearyl alcohol, mink oil, cetyl alcohol, oleyl alcohol, isopropyl laurate, polyethylene glycol, olive oil, petroleum jelly, palmitic acid, oleic acid, and myristyl myristate.

Surfactants can include polysorbates, sorbitan esters, poloxamer, or sodium lauryl sulfate. In one embodiment, the surfactant is sodium lauryl sulfate.

Metal chelators can include physiologically acceptable chelating agents including edetic acid, malic acid, or fumaric acid. In one embodiment, the metal chelator is edetic acid.

pH adjusters can also be utilized to adjust the pH of a solution containing the immunophilin ligand to about 4 to about 6. In one embodiment, the pH of a solution containing the immunophilin ligand is adjusted to a pH of about 4.6. pH adjustors can include physiologically acceptable agents including citric acid, ascorbic acid, fumaric acid, or malic acid, and salts thereof. In one embodiment, the pH adjuster is citric acid.

Fillers that can be used according to the present invention include anhydrous lactose, microcrystalline cellulose, mannitol, calcium phosphate, pregelatinized starch, or sucrose. In one embodiment, the filler is anhydrous lactose. In another embodiment, the filler is microcrystalline cellulose.

In one embodiment, compositions containing the immunophilin ligand are delivered orally by tablet, caplet or capsule, microcapsules, dispersible powder, granule, suspension, syrup, elixir, and aerosol. Desirably, when compositions containing the immunophilin ligand are delivered orally, delivery is by tablets and hard- or liquid-filled capsules. In another embodiment, the compositions containing the immunophilin ligand can be delivered intravenously, intramuscularly, subcutaneously, parenterally and intraperitoneally in the form of sterile injectable solutions, suspensions, dispersions, and powders which are fluid to the extent that easy syringe ability exits. Such injectable compositions are sterile and stable under conditions of manufacture and storage, and free of the contaminating action of microorganisms such as bacteria and fungi. In a further embodiment, compositions containing the immunophilin ligand can be delivered rectally in the form of a conventional suppository. In another embodiment, compositions containing the immunophilin ligand can be delivered vaginally in the form of a conventional suppository, cream, gel, ring, or coated intrauterine device (IUD).

In another embodiment, compositions containing the immunophilin ligand can be delivered via coating or impregnating of a supporting structure, i.e., a framework capable of containing of supporting pharmaceutically acceptable carrier or excipient containing a compound of the invention, e.g., vascular stents or shunts, coronary stents, peripheral stents, catheters, arterio-venous grafts, by-pass grafts, and drug delivery balloons for use in the vasculature. In one embodiment, coatings suitable for use include, but are not limited to, polymeric coatings composed of any polymeric material in which the compound of the invention is substantially soluble. Supporting structures and coating or impregnating methods, e.g., those described in U.S. Pat. No. 6,890,546, are known to those of skill in the art and are not a limitation of the present invention.

In yet another embodiment, compositions containing the immunophilin ligand can be delivered intranasally or intrabronchially in the form of an aerosol.

Solutions or suspensions of these active compounds as a free base or pharmacologically acceptable salt are prepared in water suitably mixed with a surfactant such as hydroxypropylcellulose. Dispersions are also prepared in glycerol, liquid, polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form is sterile and fluid to the extent that easy syringe ability exits. It is stable under conditions of manufacture and storage and is preserved against the contaminating action of microorganisms such as bacterial and fungi. The carrier is a solvent or dispersion medium containing, for example, water, ethanol (e.g., glycerol, propylene glycol and liquid polyethylene glycol), suitable mixtures thereof, and vegetable oil.

The present invention also provides kits or packages containing the immunophilin ligands. Kits of the present invention can include the ligand and a carrier suitable for administration to a mammalian subject as discussed above. The kits can also contain the reagents required to prepare the immunophilin ligands. Also within the scope of the invention are kits comprising the complexes, components thereof, and/or reagents and instructions for use.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

### Example 1

### Synthesis of Rapamycin Analogues I and II

The complexes of FK506 and rapamycin with their respective protein targets result in immunosuppressive activity that may be undesirable in the context of a therapy for chronic neurodegeneration (Lam et al, J. Biol. Chem. 270, 26511-22 (1995)). Therefore, to develop non-immunosuppressive immunophilin ligands, rapamycin analogues I and II were prepared from rapamycin via a [4+2] cycloaddition reaction with nitrosobenezene at the C1,C3 diene in order to disrupt the interaction with mTOR while leaving the FKBP binding portion intact (FIG. 1A) as described in more detail below.

### Synthesis of Rapamycin Analogue I

Chemicals were purchased from Sigma-Aldrich (St. Louis, Mo.).

Rapamycin (0.3 g, 0.328 mmol) was dissolved in 5 mL toluene with gentle heating. To this solution was added, dropwise, a solution of nitrosobenzene (0.1 g, 3 eq) in 5 mL toluene. The reaction mixture was stirred at 70° C. for 16 hours, and then the products were chromatographed via reversed-phase high performance liquid chromatography (HPLC) (column: 250×20 mm YMC ODS-A with 50×20 guard, mobile phase: 80 to 85% methanol:water in 40 minutes, flow=20 mL/min) to yield 0.139 g of the product (42% yield, t_{R}=12.1 min, analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min). ¹H-NMR (500 MHz, CD₃CN): δ7.29 (m, 2H, H57), 7.02 (m, 2H, H56), 6.90 (m, 1H, H58), 6.25 (m, 1H, H2), 5.65 (m, 1H, H43), 5.25 (m, 1H, H29), 5.16 (m, 1H, H5), 5.12 (m, 1H, H25), 5.07 (m, 1H, H4), 4.37 (m, 1H, H22), 4.09 (m, 1H, H31), 3.95 (m, 1H, H32), 3.74 (m, 1H, H9), 3.69 (m, 1H, H1), 3.59 (m, 1H, 31-OH), 3.44 (m, 1H, H28), 3.44 (m, 1H, H28), 3.33 (s, 3H, Me54), 3.29 (m, 3H, Me53), 3.27 (m, H4, H42), 3.07 (m, 1H, H34), 3.06 (s, 3H, Me52), 2.94 (m, 1H, H18), 2.86 (m, 1H, H41), 2.84 (m, 1H, H26), 2.64 (m, 1H, H26′), 2.14 (m, H4, H21), 2.09 (m, 1H, H12), 2.05 (m, 1H, H40), 2.01 (m, 1H, H36), 2.00 (m, 1H, H35), 1.87 (m, 1H, H37), 1.85 (m, 1H, H43), 1.81 (m, 1H, H21′), 1.78 (s, 3H, Me48), 1.74 (m, 1H, H19′), 1.74 (m, 1H, H20), 1.69 (m, 1H, H8), 1.64 (m, 1H, H44), 1.63 (m, 1H, H8′), 1.60 (m, 1H, H11), 1.55 (m, 1H, H44′), 1.51 (s, 3H, Me45), 1.43 (m, 2H, H10), 1.42 (m, 1H, H19′), 1.39 (m, 1H, H20′), 1.37 (m, 1H, H39), 1.27 (m, 1H, H38), 1.12 (d, 3H, Me50), 1.06 (d, 311, Me47), 1.04 (m, 1H, H38′), 1.03 (d, 3H, Me49), 0.89 (d, 311, Me51), 0.83 (d, 3H, Me46), 0.63 (m, 1H, H40′); ¹³C-NMR (125 MHz, CD₃CN): δ215.4 (s, C33), 209.5 (s, C27), 198.5 (s, C15), 170.6 (s, C23), 166.4 (s, C16), 149.2 (s, C55), 139.9 (s, C6), 138.7 (s, C30), 130.0 (d, C57), 128.0 (d, C3), 127.9 (d, C29), 127.2 (d, C5), 127.0 (d, C2), 121.5 (d, C58), 116.1 (d, C56), 99.5 (s, C13), 87.3 (d, C32), 85.2 (d, C41), 84.8 (d, C7), 78.2 (d, C31), 77.0 (d, C25), 74.5 (d, C42), 68.4 (d, C4), 68.3 (d, C9), 60.3 (d, C1), 58.6 (q, C53), 57.4 (d, C22), 56.9 (q, C54), 56.1 (q, C52), 46.9 (d, C28), 42.8 (d, C34), 41.7 (t, C26), 39.5 (t, C18), 39.5 (t, C8), 38.6 (t, C35), 38.5 (t, C38), 37.5 (d, C36), 35.6 (d, C12), 35.3 (t, C40), 33.9 (d, C37), 33.8 (d, C39), 32.9 (t, C43), 32.2 (t, C10), 32.2 (t, C44), 28.2 (t, C21), 27.7 (t, C11), 25.1 (t, C19), 21.6 (t, C20), 18.5 (q, C50), 18.0 (q, C49), 16.7 (q, C51), 16.3 (q, C46), 16.0 (q, C47), 12.4 (q, C48), 10.8 (q, C45); FT-ICRMS (m/z): [M+H]⁺ calc for C₅₇H₈₅N₂O₁₄, 1021.59954; found, 1021.59780.

Synthesis of Rapamycin Analogue II

Rapamycin analogue I (0.29 g, 0.284 mmol) was dissolved in 7 mL methanol in an 18 mm test-tube, and a spatula tip of Pd/C catalyst (Aldrich) was added. The mixture was hydrogenated on a Parr apparatus for 15 minutes at 2.0 atmosphere H₂. The products were chromatographed via reversed-phase HPLC (column 250×20 mm YMC ODS-A with 50×20 guard, mobile phase: 80% methanol:water for 15 minutes, then to 85% in 5 minutes, then held at 85% for 20 minutes, flow=20 mL/min) to yield 0.089 g of the product (31% yield, t_{R}=12.6 min, analytical HPLC conditions: column=YMC ODS-A S-3 120 Å, mobile phase/gradient: 95% water (+0.025% formic acid)/acetonitrile (+0.025% formic acid) to 5% water in 6 minutes, hold at 5% for 9 minutes, flow=0.30 mL/min). ¹H-NMR (500 MHz, CD₃CN): δ7.25 (m, 2H, H57), 6.91 (m, 2H, H56), 6.79 (m, 1H, H58), 5.44 (m, 1H, H29), 5.35 (m, 1H, H5), 5.24 (m, 1H, H25), 5.11 (m, 1H, H22), 4.50 (m, 1H, H4), 4.42 (m, 1H, 13-OH), 4.00 (m, 1H, H31), 3.80 (m, 1H, H9), 3.77 (m, 1H, H32), 3.67 (m, 1H, H7), 3.57 (m, 1H, 31-OH), 3.43 (m, 1H, H28), 3.35 (m, 1H, H18), 3.35 (s, 3H, Me54), 3.34 (m, 1H, H1), 3.32 (m, 1H, H18′), 3.32 (s, 3H, Me53), 3.27 (m, 1H, H42), 3.16 (m, 1H, H34), 3.08 (s, 3H, Me52), 3.00 (m, 1H, 42-OH), 2.87 (m, 1H, H41), 2.79 (m, 1H, H26), 2.71 (m, 1H, H26′), 2.29 (m, 1H, H21), 2.18 (m, 1H, H36), 2.10 (m, 1H, H40), 1.95 (m, 1H, H35), 1.95 (m, 1H, H37), 1.86 (m, 1H, H43), 1.85 (m, 1H, H2), 1.85 (m, 1H, H3), 1.82 (m, 1H, H12), 1.79 (m, 1H, H2′), 1.77 (m, 1H, H₂O), 1.71 (m, 1H, H8), 1.69 (m, 1H, H19), 1.68 (m, 1H, H21′), 1.66 (s, 3H, Me48), 1.64 (m, 1H, H44), 1.63 (m, 1H, H8′), 1.61 (m, 1H, H10), 1.60 (m, 2H, H11), 1.50 (m, 1H, Me45), 1.46 (m, 1H, H3′), 1.43 (m, 1H, H19′), 1.39 (m, 1H, H20), 1.39 (m, 1H, H39), 1.35 (m, 1H, H10′), 1.29 (m, 1H, H38), 1.26 (m, 1H, H43′), 1.13 (d, 3H, Me47), 1.12 (m, 1H, H38′), 1.07 (d, 3H, Me49), 1.03 (m, 1H, H35′), 1.03 (d, 3H, Me46), 1.00 (m, 1H, H44′), 0.97 (d, 3H, Me50), 0.91 (d, 3H, Me51), 0.66 (m, 1H, H40′); ¹³C-NMR (125 MHz, CD₃CN): δ216.1 (s, C33), 210.3 (s, C27), 198.3 (s, C15), 170.3 (s, C23), 168.3 (s, C16), 149.9 (s, C55), 139.9 (s, C30), 139.4 (s, C6), 130.2 (d, C57), 129.4 (d, C5), 128.1 (d, C29), 119.7 (d, C58), 114.2 (d, C56), 98.4 (s, C13), 88.5 (d, C32), 85.4 (d, C41), 85.0 (d, C7), 77.7 (d, C31), 76.3 (d, C25), 74.8 (d, C42), 72.3 (d, C4), 68.5 (d, C9), 60.0 (d, C1), 59.2 (q, C53), 57.1 (q, C54), 56.0 (q, C52), 52.0 (d, C22), 46.5 (d, C28), 45.1 (t, C18), 42.7 (d, C34), 42.1 (t, C26), 40.8 (t, C35), 39.1 (t, C38), 38.3 (t, C8), 35.7 (t, C40), 35.0 (d, C12), 34.3 (d, C37), 34.1 (d, C39), 33.1 (t, C43), 32.5 (t, C44), 32.1 (t, C10), 32.0 (d, C36), 29.1 (t, C11), 28.0 (t, C21), 26.8 (t, C3), 25.9 (t, C19), 21.7 (t, C20), 20.6 (t, C2), 19.0 (q, C49), 17.5 (q, C47), 17.4 (q, C50), 16.8 (q, C46), 16.4 (q, C51), 13.1 (q, C48), 10.4 (q, C45); FT-ICRMS (m/z): [M+H]⁺ calc for C₅₇H₈₇N₂O₁₄, 1023.61519; found, 1023.61722.

### Biological Activities of Rapamycin Analogues I and II

### Methods

### Neurite Outgrowth Measurements

Cortical neurons were fixed using 2% paraformaldehyde for 5 min followed by 4% paraformaldehyde for 5 min. Cells were incubated in blocking solution (0.2% Triton-X+1.5% normal goat serum in PBS) followed by primary (anti-neuronal class III β-tubulin (TUJ1) (Covance Innovative Antibodies, Berkeley, Calif.) and secondary antibody (Alexa Fluor 488 goat anti-mouse) (Molecular Probes, Carlsbad, Calif.). Each step was performed at room temperature for 1 hr. Total neurite outgrowth for each condition was analyzed using the Neuronal Profiling Bioapplication on an ArrayScan HCS Reader (Cellomics, Pittsburgh, Pa.).

### Neuronal Survival Assay (Neurofilament ELISA)

Cultures were fixed for 30 min with 4% paraformaldehyde at 37° C. Nonspecific binding was blocked by incubating with PBS containing 0.3% Triton X-100 and 5% fetal bovine serum (FBS) for 45 min. Cultures were then incubated overnight at 4° C. with an anti-neurofilament (200 kD) monoclonal antibody (1:1000, clone RT-97, Chemicon, Temecula, Calif.). After washing, a peroxidase-conjugated secondary antibody (1:1000, Vector Labs, Burlingame, Calif.) was applied for 2 h. After three washes, the peroxidase substrate K-BlueMax (Neogen, Lexington, Ky.; Young et al., 1999) was added to the cultures and incubated for 10 min on an orbital shaker. The peroxidase substrate is highly soluble in the K-BlueMax solution. Optical density is then readily measured using a Molecular Devices Spectramax Plus colorimetric plate reader at 650

### Immunosuppression Assay

Human CD4⁺ T cells were purified by negative selection from peripheral blood lymphocytes using RosetteSep as per manufacture's instructions (StemCell Technologies, Inc. Vancouver, British Columbia). Tosyl-activated magnetic microspheres (Dynal, Great Neck, N.Y.) were coated with anti-CD3 Ab (1 μg/10⁷ microspheres), and anti-CD28Ab (0.5 m/10⁷ microspheres) as described in Blair et al. J. Immunol., 160:12, 1998. Murine IgG was used to saturate the binding capacity of the microspheres (total protein=5 μg/10⁷ microspheres). Protein-coated microspheres were added to purified CD4+T cells (2×10⁶cells/mL, ratio 1 bead: 1 cell) and activated for 72 hours in RPMI, 10% fetal calf serum, 2 mM glutamine media. Cells were harvested, washed, and cultured overnight in fresh media and re-stimulated with IL-2 as described in Bennett et al., J. Immunol. 170:711, 2003. Briefly, overnight rested cells were recounted, plated (10⁵ cells/well) in flat-bottomed 96 well microtiter plates and stimulated with 1 ng/mL human IL-2 (R&D Systems, Minneapolis, Minn.) in the presence of increasing concentrations of compound. Seventy-two hours after culture re-stimulation, plates were pulsed with 1 μCi/well tritiated thymidine and incubated for a 6-16 hour period.

### Results

As described above, rapamycin analogs I and II were prepared from rapamycin via a [4+2] cycloaddition reaction with nitrosobenezene at the Cl, C3 diene in order to disrupt the interaction with mTOR while leaving the FKBP binding portion of the compound intact. (FIG. 1A). Compound II showed no detectable inhibition of IL-2 stimulated CD4+T-cell proliferation up to 1 μM, in contrast to rapamycin (IC₅₀=0.005 μM). Moreover, Compound I was found to promote neuronal survival, as measured by neurofilament ELISA, in cultured rat cortical neurons (FIG. 1B), and to promote neurite outgrowth in both cortical neurons (FIG. 1C) and F-11 cells (FIG. 1D). Importantly, 10 and 30 mg/kg of Compound 2 significantly reduced infarct volume by 24% and 23%, respectively, in a transient mid-cerebral artery occlusion model for ischemic stroke (see Example 9 of U.S. Ser. No. 06/0135549). Given the therapeutic potential of these compounds, the cellular target(s) of these compounds were identified to evaluate their roles in promoting neuronal survival and neurite outgrowth.

### Example 2

### Chemical Synthesis and Preparation of Affinity Matrix

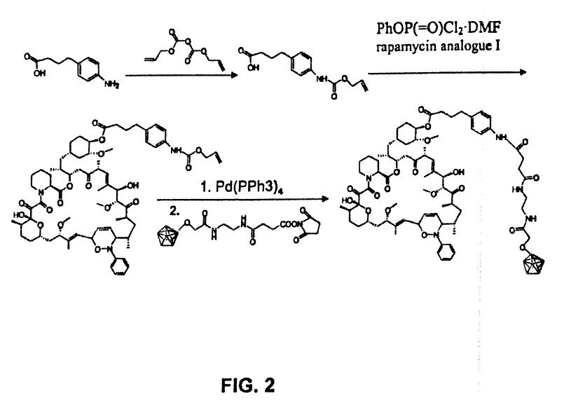

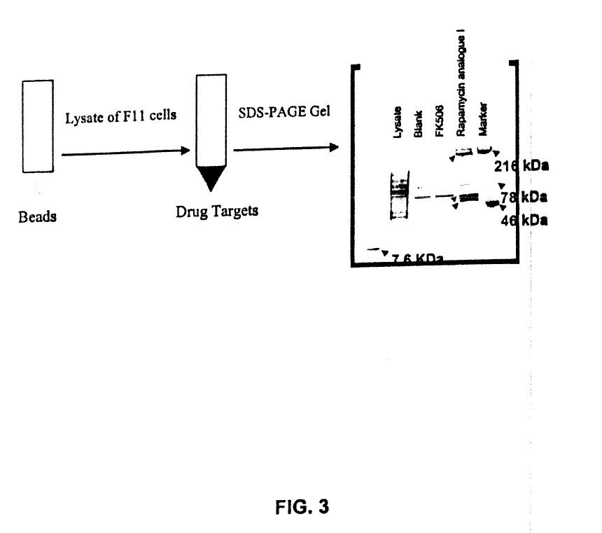

To identify the target proteins, affinity matrices containing rapamycin analogue I, rapamycin analogue II and the meridamycin analogue were prepared by linking the compound to Affi-Gel 10 resin through amino-phenyl-butyric acid (FIG. 2) according to the methods published by Fretz et al. supra. Briefly, the amino group of amino-phenyl-butyric acid (1200 mg) was protected with an allyloxycarbonyl group by treating with diallyldicarbonate (1200 μM) in dioxane:water (3:1; 50 ml) for 3 h at room temperature.

The acid group of the resulting 4-(para-N-Alloc-aminophenyl) butanylester (80 mg) was activated by PhOP(O)Cl₂.DMF complex in CH₂Cl₂ (1 ml) at 4° C., and reacted with the 42-hydroxyl group of the rapamycin analogue I (80 mg) in the presence of pyridine (90 μM) at room temperature for 30 min. The reaction was quenched with methanol and the ester product was purified by HPLC with a purity of 99% and characterized by MS and NMR. After removing the allyloxycarbonyl group of the ester product (40 mg) by treatment with Pd(PPh₃)₄ (2 mg) and dimedone (7 mg) in THF (1.2 ml), the amino group of the product was linked to Affigel-10 matrix (4 ml) in the presence of 2% pyridine in THF. The resulting Affi-Gel-rapamycin analogue I affinity matrix was washed with ethanol, water and ethanolamine 50 mM Hepes pH 8.0 buffer and stored in 40% ethanol.

Similar approaches were used to prepare affinity matrix containing rapamycin analogue II, a meridamycin analogue disclosed as compound I in U.S. 2005/0197379, FK506 and rapamycin.

### Example 3

### Affinity Precipitation of Target Proteins

The matrices prepared in Example 2 were used to precipitate target proteins from the lysates of F-11 (a hybrid of rat dorsal root ganglia neurons (DRG) and mouse neuroblastoma) cells (Platika, D. et al. (1985) Proc. Natl. Acad. Sci. USA 82:3499-3503).

Experiment A.

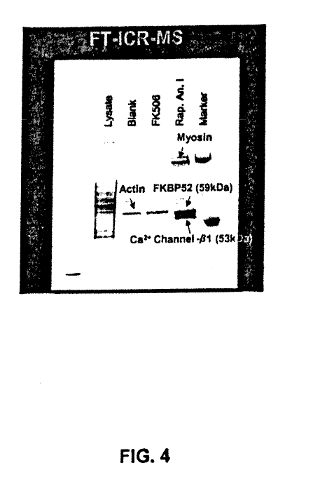

F11 cells were grown in culture medium, DMEM supplemented with 10% FBS and 1% pen/Strep, in 75 cm² vented flasks in 37° C. incubator with 5% CO₂. Cells were harvested at 80% confluence and washed with PBS buffer. Lysis buffer (6 ml; 50 mM Tris, pH 7.4, 250 mM NaCl, 5 mM EDTA, 50 mM NaF, 1 mM Na₃VO4, 1% Nonidet P40 (NP40), 0.1% mercaptoethanol and 2% protease inhibitor cocktails) was added to 10⁹ cells. Cells were broken by forcing them through a 26-gauge needle, and S-100 supernatant was collected after 15 min centrifugation at 4° C. Aliquots (2 ml) were mixed with affinity beads (100-150 μl; such as Affigel10, Affigel10-FK506 and Affigel10-rapamycin analogue I) at 4° C. overnight. After washes with lysis buffer (2 ml) and then PBS (2 ml), the beads were analyzed on 4-20% SDS-PAGE gel. FIG. 2 shows the following lanes: lysate of F11 cells, blank (proteins bind to Affigel-10 beads), FK506 (proteins bind to Affigel-10-FK506 beads), rapamycin analogue II (proteins bind to Affigel-10-rapamycin analogue I beads), marker (protein standards). The protein bands (FIG. 3) were cut out and digested with trypsin (0.3 ng) in digestion buffer (30 μl; 0.2% NH4HCO3) at 30° C. overnight. The resulting peptides were purified on C18-resin and submitted for FT-ICR-MS analysis. The FT-ICR-MS data was manually edited and used to search protein databases. The results are shown in FIG. 4 and have the following scores. FK506-binding protein (FKBP52) (P30416, score: 94, expect: 9.6e-05); MS Data of the 59 kDa band: 2753.35; 1710.94; 2215.13; 2363.15; 1298.71; 1215.59; 1000.51; 1000.46; 1790.93; 1381.70; 2746.36; 1316.71; and 1171.60. Voltage dependent L-type calcium channel β1 subunit (Q8R3Z5-03-00-00, score: 133, expect: 1e-09); MS Data of the 52 kDa band: 651.38; 663.39; 779.54; 853.55; 1014.50; 1347.75; 1217.78; 1346.67; 1297.75; 1231.77; 877.52; 853.47; 919.48; 1014.56; 1041.63; 1217.74; 1231.74; 1296.84; 869.58 (major).

Thirteen fragments of the ˜60 kDa band matched the partial sequence of the FKBP52 protein with a p-value of 9.6e-5, and 19 fragments of the ˜50 kDa band matched the partial sequence of the β1 subunit of the voltage gated L-type calcium channel (CACB1). Other minor components were skeleton proteins (actin and myosin).

Therefore, immunophilin FKBP52 and CACB1 were identified as binding candidates for rapamycin analogue I.

Experiment B.

In another experiment, F11 cells were grown in culture medium, DMEM supplemented with 10% FBS and 1% Pen/Strep, in 75 cm² vented flasks in a 37° C. incubator with 5% CO₂. Cells were harvested at 80% confluence and washed with PBS buffer. To 3×10⁸ cells, lysis buffer (2 ml; 50 mM Tris, pH 7.4, 250 mM NaCl, 5 mM EDTA, 50 mM NaF, 1 mM Na₃VO₄, 1% Nonidet P40, 0.1% mercaptoethanol and 2% protease inhibitor cocktails) was added, and its S-100 supernatant was collected after 15 min centrifugation at 4° C. Aliquots (2 ml) were incubated with affinity beads (100-150 μl) at 4° C. After wash with lysis buffer (2 ml) and then PBS buffer (2 ml), beads were analyzed by SDS-PAGE. The protein bands were cut and digested with trypsin (0.3 μg) in digestion buffer (30 μl; 0.2% NH₄HCO₃) at 30° C. The resulting peptides (2 μl) were loaded into a nanoelectrospray tip of FT-ICR-MS and mixed with 1% formic acid in methanol (2 μl). A high voltage about −800 V was applied between the nanoelectrospray tip and the glass capillary. The resulting mass spectra data were externally calibrated using HP tuning mix, and used for Mascot search in NCBI protein databases. Reasonable protein candidates were selected based on confident scores (p value). For Western analysis, the precipitated proteins were separated on by SDS-PAGE, transferred to PVDF membranes by electroblotting (100V, 1 hr), immunoblotted with the anti-CACNB1 or anti-FKBP4 antibody, and visualized by 3,3′,5,5′-tetramethylbenzidine (TMB) staining.

### Results

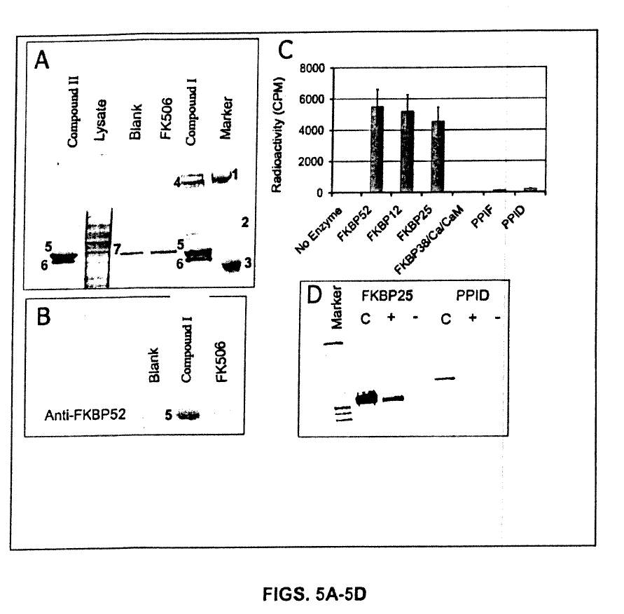

As shown in FIG. 5A, three strong bands (220 kDa, 60 kDa and 50 kDa) and two very weak bands (25 kDa and 12 kDa) were found in both rapamycin analogue I and II pull-down fractions. FT-ICR-MS spectra of each band were used for Mascot search in the NCBI database (see Table 1 below). FKBP52 (Gold, B. G. Drug Metab. Rev. 31, 649-663 (1999)) and the β1 subunit (CACNB1) of the voltage gated L-type calcium channel (VGCC) (Opatowsky, Y. et al. Neuron 42, 387-399 (2004). Structural analysis of the voltage-dependent calcium channel beta subunit functional core and its complex with the alpha 1 interaction domain were identified as major targets of rapamycin analogue I and II, and their presence was confirmed by Western analysis (FIG. 5B). FKBP25 and FKBP12 were identified in the weak bands, whereas myosin and actin were found in all fractions, indicating non-specific binding to the resin.

TABLE 1
FT-ICR-MS analysis of proteins that bind to rapamycin analogues I and II

| MS Data | Identified protein |
|---|---|
| 2753.35, 1710.94, 2215.13, 2363.15, 1298.71, 1215.59, 1000.51, | Major band, 59 kDa |
| 1000.46, 1790.93, 1381.70, 2746.36, 1316.71, 1171.60 | FKBP52, P = 1e−09 |
| 651.38, 663.39, 779.54, 853.55, 1014.50, 1347.75, 869.58 (major), | Major band, 52 kDa |
| 877.52, 853.47, 919.48, 1014.56, 1041.63, 1217.74, 1231.74, | CACNB1, P = 1e−09 |
| 1296.84, 1346.67 | |
| 616.32, 701.42, 802.42, 888.15, 908.97, 980.48, 1010.56, 1132.55, | Minor band (<5%), 25 kDa, |
| 1405.67, 1424.71, 1611.90, 1764.81, 2328.12, 2366.15, 2342.22, | FKBP25, P = 3.8e−12 |
| 2365.15, 2442.22, 2458.17, 2493.20, 2525.20, 3101.49, 3277.66, | |
| 3235.66, 3275.65, | |
| 739.47, 881.58, 1190.68, 1314.66, 1011.69, 1533.69, 903.6 | Minor band (<5%), 12 kDa, |
| | FKBP12, P = 0.03 |

### Example 4

### Characterization of the Precipitated Targets by Western and Kinetic Analysis

### Methods

Cloning and Expressing Recombinant Genes and Binding Assays.

Using Gateway cloning methods developed by Invitrogen (Carlsbad, Calif.), cacnb1/CACNB 1, cacnb4/CACNB4fkbp3/FKBP25, fkbp4/FKBP52, ppid, ppif and fkbp8/FKBP38 genes were cloned into the pDEST17 (N-His₆ tag) vector. The His₆-CACNB1: TGG548TAA were generated from pDEST17-CACNB1 using QuikChange site-directed mutagenesis kit (Stratagene, LaJolla, Calif.). The His₆ tagged protein was purified on a Ni-NTA column (Qiagen, Valencia, Calif.). Proteins showed above 95% purity by SDS-PAGE analysis, and were used fresh. FKBP38 was tested in the presence of 2 mM Ca2′ and 5 μM CaM (Edlich, F. et al. J. Biol. Chem. 281, 14961-14970 (2006). The binding to rapamycin analogue II was measured by SDS-PAGE based on the amount of proteins retained on rapamycin analogue II matrix in comparison with blank Affi-Gel 10 beads. The binding of rapamycin analogue I was measured by quantifying the ¹⁴C radioactivity coeluted with the protein through TopTip P-4 column, after reacting each purified protein (10 μM) with [¹⁴C]-rapamycin analogue I (10 μM, 241 Ci/mol) at 37° C. The protein fluorescent quenching induced by rapamycin analogue I was measured by titrating His₆-CACNB1:TGG548TAA protein (0-8 μM) with rapamycin analogue I (1 μM).

### Kinetic Analysis

Binding of immunophilin ligands to His₆-tagged FKBP12 and FKBP52 proteins was measured by quantitation of ³H FK506 retained on Ni-chelated FLASH plate in 0.1 ml reaction mixtures containing 50 mM Hepes, pH 7.4, 0.1% Tween-20, (0-10 μM) immunophilin ligands, 3 nM [³H]-FK506 (87 Ci/mmol), and (5 nM) enzyme. Reactions were carried out in triplicate at 25° C. for 30 min. K_{d} were calculated using methods described by Carreras (Anal. Biochem. 298, 57-61 (2001)).

The following materials used in the examples described herein were obtained from the following commercially available sources: Antibodies were from Abcam (Cambridge, Mass.). Media, human ORF clones (cacnb1, cacnb4, fkbp3, fkbp4, fkbp8, ppiF, and ppiD), plasmids (pDEST17), and SUPERSCRIPT® System were from Invitrogen (Carlsbad, Calif.). Protein purification kits were from Pieres (Rockford, Ill.) or Qiagen (Valencia, Calif.). TOPTip P-4 column was from Glygen (Columbia, Md.). Ni-chelated Flash plates and [³H]-FK506 were from PerkinElmer Life Science (Boston, Mass.). PCR reagents and Affi-Gel 10 were from BioRad (Hercules, Calif.). Rat Genome 230 2.0 GENECHIP® is from AFFYMETRIX® (Santa Clara, Calif.). FT-ICR-MS analysis was carried out on a Bruker (Billerica, Mass.) APEXII FT-ICR mass spectrometer equipped with an actively shielded 9.4 Tesla superconducting magnet (Magnex Scientific Ltd., UK), and an external Bruker APOLLO ESI source.

### Results

Table 2 shows that both FK506 and rapamycin bind to FKBP12 and FKBP52 with comparable affinities (K_{d}(FKBP12)/K_{d}(FKBP52)=0.46 and 0.23 respectively). In contrast, Compound 2 showed a marked preference of binding to FKBP52 relative to FKBP12 (K_{d}(FKBP12)/K_{d}(FKBP52)=229). This is unexpected because Compound 2 has the same pipecolate moiety for FKBP binding as rapamycin, and the site of modification is distant. X-ray structures have shown that the isomerase domains of FKBP52 and FKBP12 are very similar (Wu et al., Proc Natl Acad Sci USA. 101, 8348-53 (2004), and sequence alignment of their active site residues showed only one amino acid difference (His87 in FKBP12 versus Ser118 in FKBP52) (Dornan et al., Curr. Top. Med. Chem. 3, 1392-1409 (2003)). Rapamycin and its analogs are known to exist as a set of major and minor solution conformers, due to rotation about the amide bond (Kessler et al., Helv. Chim. Acta 76, 117-130 (1993)). The additional moiety NO-phenyl moiety affects the overall global population of macrolactone conformers, which in turn affects immunophilin selectivity. This observation appears consistent with the dramatic differences in binding affinities for Compound 2 towards different yet homologous immunophilins appears consistent with the dramatic differences in binding affinity reported for FKBP25 between rapamycin and FK506 (Galat et al., Biochemistry 31, 2427-2434 (1992)). This shows non-scaffold modifications to rapamycin that enhance binding to specific FKBPs.

To further validate the specificity of the compounds for immunophilins and the related cyclophilins, the binding of Compound 1 and Compound 2 to purified recombinant FKBP25, FKBP38, cyclophilin F (PPID), cyclophilin D (PPIF) was measured. These targets were chosen, in light of the in vivo activity of Compound 2 (vide infra), because of their reported importance in stroke models (Edlich et al., J. Biol. Chem. 281, 14961-14970 (2006); Baines et al., Nature 434, 658-662 (2005); Edlich et al., EMBO J. 24, 2688-2699 (2005)). The binding results of {¹⁴C}-1 to the various putative targets are shown in FIG. 5C. At a 10 μM concentration, [¹⁴C]-1 binds to FKBPs well, PPID weakly, and PPIF and FKBP38/Ca²⁺/CaM negligibly. Compound 2 also binds to FKBP52, FKBP25 and FKBP12 with a similar selectivity profile (FIG. 5D, Table 2).

TABLE 2
Binding of immunophilin ligands to FKBP12 and FKBP52

| Compounds | FKBP 12 (K_{d}, nM) | FKBP 52 (K_{d}, nM) |
|---|---|---|
| FK506 | 0.33 ± 0.03 (Lit. 0.4³⁰) | 0.72 ± 0.07 |
| Rapamycin analogue II | 110 ± 11 | 0.48 ± 0.04 |
| Rapamycin analogue I | 4.7 ± 0.4 | 0.55 ± 0.05 |
| Rapamycin | 0.33 ± 0.03 | 1.4 ± 0.1 |
| GPI-1046 | >110 | >12 |

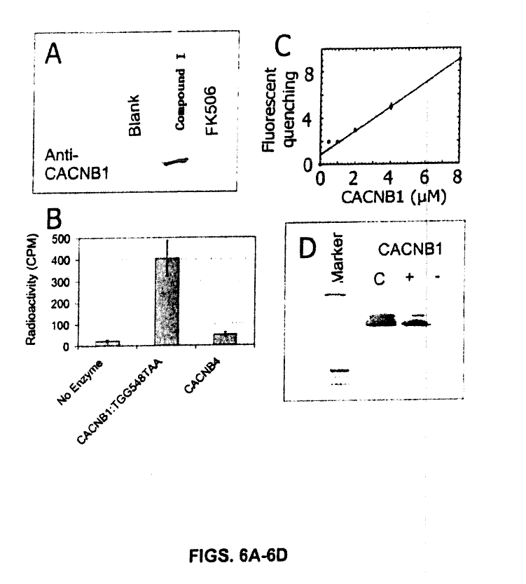

The other major binding protein identified in the affinity purification and confirmed by Western analysis (FIG. 6A), CACNB1, is one of the β subunits associated with the L-type Ca²⁺ channels in primary neurons. To further validate this specific subunit as a binding partner for Compounds 1 and 2, binding to the P4 subunit (CACNB4) of the VGCC and C-terminal truncated CACNB1 was determined (FIG. 6B). Recombinant His₆-CACNB1:TGG548TAA protein was prepared by removing 51 C-terminal residues from CACNB1. Binding to full length CACNB4 was also tested because of its sequence homology to CACNB1 (Opatowsky, Y. et al. Neuron 42, 387-399 (2004)). At a 10 μM concentration, [¹⁴C]-Compound 1 binds to the mutant His₆-CACNB1:TGG548TAA weakly and CACNB4 negligibly. To further confirm the binding of Compound 1 to the His₆-CACNB1:TGG548TAA mutant, we measured protein fluorescent quenching induced by Compound 1. FIG. 6C shows a linear dose response curve, indicating binding of Compound 1 to CACNB1. Compound 2 also binds to CACNB1 with a similar selectivity profile (FIG. 6D)

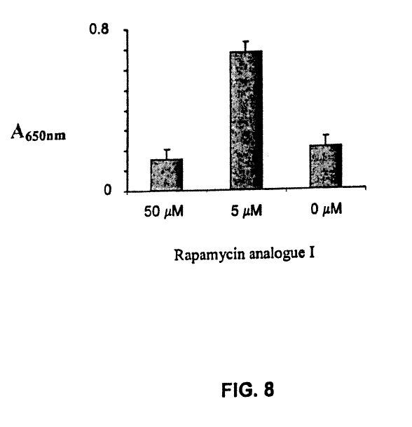

The existence of the drug targets or binding candidates for rapamycin analogue I (immunophilin FKBP52 and CACB1) was also confirmed by Western blotting using the corresponding antibodies. The proteins on the affinity beads were separated by 4-20% SDS-PAGE gel, and transferred to PVDF membrane at 100 V for 1 h. The membranes were blotted with blocking solution, primary antibody (anti-FKBP52 or anti-Ca²⁺ channel-β1 subunit antibodies; 1:200 dilution), and secondary antibody (peroxidase conjugated anti-rabbit IgG antibody; 1:1000 dilution). The existence of the target proteins was visualized after TMB staining, as shown in FIG. 8.

Western analysis demonstrated the binding of both proteins to rapamycin analogue I, but not to the blank beads. FKBP52 was detected in the fractions of rapamycin analogue I beads and FK506 beads, but not in the blank beads. The voltage dependent L-type calcium channel β1 subunit was only detected in the fraction of the rapamycin analogue I beads. This indicates that rapamycin analogue I specifically bound to FKBP52 and the β1 subunit of the voltage gated L-type calcium channel.

### Example 5

### Formation of a Novel Complex, FKBP52-Rapamycin Analogue 1-Ca²⁺ Channel β1 Subunit

Co-immunoprecipitation was used to investigate the complex formation among FKBP52, rapamycin analogue I and the voltage gated calcium channel β1 subunit. Briefly, aliquots (1.8 ml) of F11 cell lysate were mixed with 0, 5, and 50 μM rapamycin analogue I, respectively, at 4° C. for 5 h. Anti-FKBP52 antibody was added at 1:200 dilution to each aliquot and incubated at 4° C. for 5 h. Protein A beads (50-100 μM) were then added to precipitate the anti-FKBP52-antibody-associated complex. The proteins immunoprecipitated on the beads were washed with PBS buffer, separated on 4-20% SDS-PAGE gel, transferred to PVDF, and immunoblotted with anti-Ca²⁺ channel β1 subunit antibody (1:500 dilution) to detect the β1 subunit.

### Results

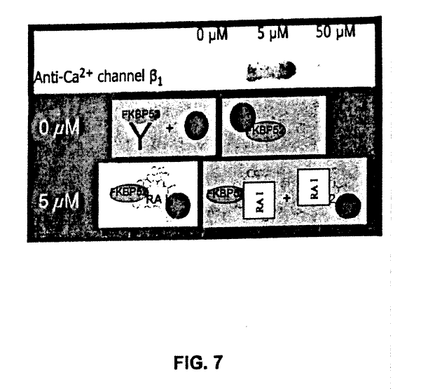

The results are shown in FIG. 7. The Ca²⁺ channel β1 subunit did not precipitate with FKBP52 in the absence of rapamycin analogue I, indicating that the Ca²⁺ channel β1 subunit does not associate with FKBP52. In the presence of rapamycin analogue I (5 μM), a large amount of Ca²⁺ channel β1 subunit was co-immunoprecipitated with FKBP52, indicating a complex formation. However, an excess amount of rapamycin analogue I (50 μM) reduced the amount of precipitated β1 subunit, indicating lower amount of complex formation, which may be caused by saturation of the compound binding sites on both FKBP52 and β1 subunit in a limited amount of lysate.

### Example 6

### Complex Formation Correlates with Neurite Outgrowth

Neurofilament ELISA was used to measure the neurite outgrowth of F11 cells grown in the absence or presence of rapamycin analogue I. Briefly, F11 cells were grown in DMEM supplemented with 10% FBS, 1% pen/Strep, and rapamycin analogue I (0, 5, or 50 μM) for 96 hrs. Cells were fixed with 4% paraformaldehyde for 30 min at 37° C. Nonspecific binding was blocked by incubating with PBS containing 0.3% Triton X-100 and 5% fetal bovine serum (FBS) for 45 min. Cultures were then incubated overnight at 4° C. with an anti-neurofilament (200 kD) monoclonal antibody (1:1000). After washing, a peroxidase-conjugated anti-mouse secondary antibody (1:1000) was applied for 2 h. After three washes, the peroxidase substrate K-BlueMax was added to the cultures and incubated for 10 min. Optical density was determined at 650 nm.

### Results

The results are shown in FIG. 8. The cells treated with 5 μM rapamycin analogue I showed 4-5 fold higher neurofilament content than those treated with 50 μM rapamycin analogue I or no compound control, indicating strong neurite outgrowth at 5 μM rapamycin analogue I. This directly correlated with the complex formation in the presence of the identical concentration of rapamycin analogue I.

### Example 7

### Evaluation of the Electrophysiological Properties of the Calcium Channel in F-11 Cells, Following Treatment with Rapamycin Analogues

Methods. Whole-Cell Patch Clamp Recordings

The whole-cell configuration of the patch-clamp technique was used to record calcium currents from the cells at room temperature using an EPC-9 amplifier (HEKA, Instrutech Corp.) with the acquisition and analysis program Pulse-PulseFit from HEKA (Lambrecht, Germany). Electrodes were fabricated using a P-87 puller (Sutter Instrument). Electrodes had a resistance of 2-5 MΩ when filled with recording solution (140 mM CsCl, 10 mM EGTA, 10 mM HEPES, 5 mM MgCl₂, 2 M ATP, 1 mM cAMP, pH 7.2). The standard bath recording solution is Ca²⁺ and Mg²⁺ free HBSS (pH 7.4) containing 10 mM HEPES, 10 mM dextrose, and 4 mM BaCl₂. Currents were filtered at 3 kHz, and the inward Ca²⁺ currents were recorded from cells held at −90 mV with 10 mV depolarizing steps from −80 mV to 60 mV for 50 ms.

### Results

CACNB1, is one of the β subunits associating with the L-type Ca²⁺ channels in primary neuron (Pichler et al., J. Biol. Chem. 272, 13877-13882 (1997)). The β1b, β3 and β4 subunits are known to enhance L-type Ca²⁺ channel current, whereas the O₂ subunit plays a negative role (Opatowsky et al, Neuron 42, 387-399 (2004); Schjott et al., J. Biol. Chem. 278, 33936-33942 (2003)). If binding of our rapalogs to β1b subunit inhibits the function of this subunit, the L-type Ca²⁺ current is expected to be reduced. Therefore, the electrophysiological properties of the Ca²⁺ channel in F-11 cells following treatment with rapamycin analogue 1 were measured.

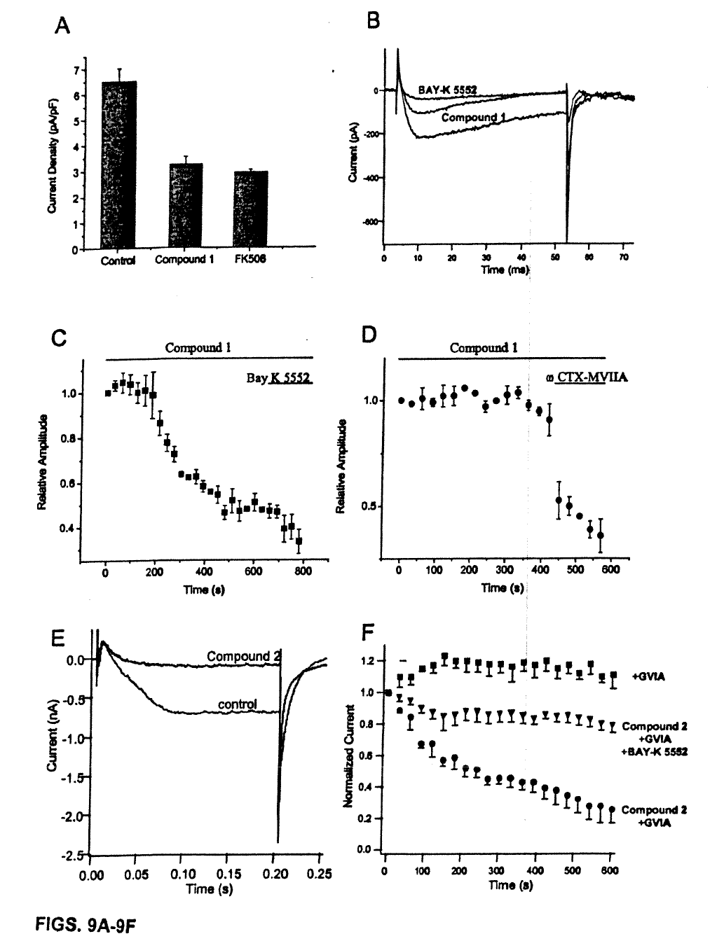

Whole-cell Ca²⁺ currents recorded in F-11 cells was not affected by bath application of Compound 1 for short time periods (10 min application; FK506 inhibited the Ca²⁺ current within this time period), so cells were exposed to 5 μM 1 for 2 hrs and then compared to vehicle treated controls. This treatment paradigm strongly reduced the Ca²⁺ currents detected in the cells, reducing the current density from 6.5+/−0.5 pA/pF to 3.2+/−0.3 pA/pF, a 49% decrease (FIG. 9A). FK506 also was found to produce a similar effect on the Ca²⁺ currents (current was reduced to 2.9+/−0.1 pA/pF, a 55% decrease), as has been described for calcineurin dependent action on Ca²⁺ currents ((Yasutsune, et al. British Journal of Pharmacology 126(3), 717-729 (1999); Fauconnier, J., et al. Am J Physiol Heart Circ Physiol. 288, H778-H786 (2005)). This, combined with the large size of Compound 1, required that for subsequent experiments, the compound be added into the cell directly by way of the recording patch pipette.

Internal application of Compound 1 via diffusion into the cell beginning when the whole-cell configuration was achieved (time 0) produced an inhibition of Ca²⁺ current immediately, reaching a steady state level of current block within several minutes. Interestingly, the compound's effect in F-11 was quite variable, but as a hybrid of DRG and neuroblastoma cells, the expression profiles of N- and L- type Ca²⁺ channels are known to differ among individual F-11 cells (Boland, L M. et al. Journal of Physiology 420, 223-245 (1990)). Some cells (FIG. 9C) contained predominantly the L-type Ca²⁺channel as determined by inhibition with BAY-K5552 (L-type blocker), while others (FIG. 9D) contained mainly the N-type channel that was inhibited by ωCTX MVIIA (N-type blocker). FIG. 9C shows that treatment with Compound 1 reduces the Ca²⁺ current, in cells responding to BAY-K5552, while FIG. 9D illustrates how cells not responding to Compound 1 contained Ca²⁺ current sensitive to ωCTX MVIIA. In the former case, internal application of Compound 1 (10 μM) reduced the Ca²⁺ current by an average of 46+/−1.8% within 10 min. (FIG. 9C). No significant current reduction was found in cells responding significantly to ωCTX MVIIA (FIG. 9D). Further validation of rapalog effects on Ca²⁺ currents was performed on cultured rat hippocampal neurons. When N-type Ca²⁺ channels were blocked and Compound 2 (10 μM) was added to the internal pipette solution, the current was slowly inhibited by 74.5+/−8.8 after 10 min (FIG. 9E,F). This effect was due at least partly to an inhibition of L-type channels, as block of both N- and L-type Ca²⁺ channels reduced the inhibition to only 21.3+/−4.4% of the remaining current in the cells (FIG. 9F).

### Example 8

### Transcriptional Profiling Following Treatment with Rapamycin Analogues

### Methods

### Transcriptional Profiling

Cortical neuron cultures were prepared from E16 rat embryos. After plating for 24 hrs, cultures were treated with 10 μM immunophilin ligands and the corresponding vehicle. After treatment for 4 hrs, 12 hrs, 24 hrs and 48 hrs, cells were lysed. Total RNA from each sample was extracted with the RNEASY® Mini Kit (QIAGEN®). Double stranded cDNA was synthesized from 2 μg of each RNA sample using the SUPERSCRIPT® System (INVITROGEN®), purified, transcribed in vitro to prepare biotinylated cRNA using T7 RNA polymerase in the presence of biotin labeled UTP and CTP. The fragmented cRNAs were hybridized to a Rat Genome 230 2.0 GENECHIP® (AFFYMETRIX®, Santa Clara, Calif.) as recommended by the manufacturer. Hybridized arrays were stained according to manufacture protocols on a Fluidics Station 450 and subsequently scanned on an AFFYMETRIX® scanner 3000. The raw data was generated using AFFYMETRIX® MAS 5.0 Software. Transcriptional profiling data were analyzed in Ingenuity.

### Results

To further analyze downstream consequences of rapamycin analogue binding, transcriptional profiling data of rat cortical neuron cultures treated with 10 μM of rapamycin analogue I or II were obtained.

Transcriptional profiling revealed overall down-regulation of Ca²⁺ signaling pathways after rapamycin analogue I or II treatment (see Table 3A). Rapamycin analogue I caused down-regulation of major plasma membrane Ca²⁺ influx channels, such as VGCC, transient receptor potential channels, N-methyl D-aspartate subtype of glutamate receptors (NMDA), and SHT3R channels. Among these channels, Ca²⁺ influx through the NMDA channel is a major event leading to apoptosis (Ghosh et al., Science 268, 239-247 (1995)). Plasminogen activator (PLAU), known to cleave the NMDA peptide and activate Ca²⁺ influx (Traynelis et al., Nat. Med. 7, 17-18 (2001)), was significantly down regulated (−40 fold by rapamycin analogue I, −10 fold by rapamycin analogue II); this is likely to reduce the Ca²⁺ influx through NMDA channels. Also, down regulation of IP3 receptor might reduce Ca²⁺ release from internal storage, and down regulation of calmodulin and calmodulin kinases (e.g. PNCK, −20 fold) would reduce the cytosolic Ca²⁺ signaling. The observed attenuation of Ca²⁺ influx and Ca²⁺ signaling pathways may be critical for the treatment of stroke and traumatic brain injury, because Ca²⁺ overload of neurons is generally considered the critical event triggering the Ca²⁺ dependent processes that eventually lead to neuronal death (Ghosh et al., Science 268, 239-247 (1995)). In addition, lowering cellular Ca²⁺ levels may suppress apoptosis by FKBP38/Ca²⁺/CaM activation of Bcl2 (Edlich et al., J. Biol. Chem. 281, 14961-14970 (2006)), or PPID associated mitochondrial permeability transition pore (Baines et al., Nature 434, 658-662 (2005)).

Significant upregulation of cholesterol biosynthesis genes (e.g. LSS, +13 fold) was observed, indicating activation of steroid receptors (Wang et al., J. Lipid Res. 47, 778-786 (2006)) (see Table 3B). Because activation of steroid receptors by FK506, steroid hormones or geldanamycin has been reported to stimulate neurite outgrowth, it is possible that binding of rapamycin analogue I and II to FKBP52 activates steroid receptors and promotes neurite outgrowth.

TABLE 3A
Calcium signaling pathway genes

| Gene | | fold change | p-value | fold change | p-value | | |
|---|---|---|---|---|---|---|---|
| Symbol | Gene Name | 2 vs DMSO | 2 vs DMSO | 1 vs DMSO | 1 vs DMSO | location | family |
| ACTA1 | actin, alpha 1, | −1.6 | <0.001 | −1.53 | <0.001 | Cytoplasm | other |
| | skeletal muscle | | | | | | |
| ACTA2 | actin, alpha 2, | 1.37 | 0.089 | 1.41 | <0.001 | Cytoplasm | other |
| | smooth muscle, | | | | | | |
| | aorta | | | | | | |
| AKAP5 | — | −1.5 | 0.17 | −2.56 | 0.003 | Plasma | other |
| | | | | | | Membrane | |
| ASPH | aspartate beta- | −3.27 | <0.001 | −3.19 | <0.001 | Cytoplasm | enzyme |
| | hydroxylase | | | | | | |
| ATP2A2 | ATPase, Ca++ | 2 | <0.001 | 1.85 | <0.001 | Cytoplasm | transporter |
| | transporting, | | | | | | |
| | cardiac muscle, | | | | | | |
| | slow twitch 2 | | | | | | |
| ATP2B1 | ATPase, Ca++ | 1.81 | 0.003 | 1.53 | 0.016 | Plasma | transporter |
| | transporting, | | | | | Membrane | |
| | plasma | | | | | | |
| | membrane 1 | | | | | | |
| ATP2B3 | ATPase, Ca++ | −1.45 | 0.019 | −1.54 | 0.009 | Plasma | transporter |
| | transporting, | | | | | Membrane | |
| | plasma | | | | | | |
| | membrane 3 | | | | | | |
| ATP2C1 | ATPase, Ca++ | −1.32 | 0.002 | −1.38 | <0.001 | Cytoplasm | transporter |
| | transporting, | | | | | | |
| | type 2C, | | | | | | |
| | member 1 | | | | | | |
| CABIN1 | calcineurin | 1.34 | 0.01 | 1.33 | 0.011 | Nucleus | other |
| | binding protein 1 | | | | | | |
| CACNA1B | calcium channel, | −1.45 | 0.001 | −1.77 | <0.001 | Plasma | ion channel |
| | voltage- | | | | | Membrane | |
| | dependent, L | | | | | | |
| | type, alpha 1B | | | | | | |
| | subunit | | | | | | |
| CACNA1C | calcium channel, | 1.82 | 0.001 | 1.48 | 0.001 | Plasma | ion channel |
| | voltage- | | | | | Membrane | |
| | dependent, L | | | | | | |
| | type, alpha 1C | | | | | | |
| | subunit | | | | | | |
| CACNA1D | calcium channel, | −1.28 | 0.21 | −1.72 | 0.017 | Plasma | ion channel |
| | voltage- | | | | | Membrane | |
| | dependent, L | | | | | | |
| | type, alpha 1D | | | | | | |
| | subunit | | | | | | |
| CACNA2 | calcium channel, | 5.73 | 0.009 | 3.47 | 0.03 | Plasma | ion channel |
| D1 | voltage- | | | | | Membrane | |
| | dependent, | | | | | | |
| | alpha 2/delta | | | | | | |
| | subunit 1 | | | | | | |
| CACNB1 | calcium channel, | −1.14 | 0.051 | −1.32 | 0.026 | Plasma | ion channel |
| | voltage- | | | | | Membrane | |
| | dependent, beta | | | | | | |
| | 1 subunit | | | | | | |
| CACNG2 | calcium channel, | −2 | <0.001 | −1.8 | <0.001 | Plasma | ion channel |
| | voltage- | | | | | Membrane | |
| | dependent, | | | | | | |
| | gamma subunit 2 | | | | | | |
| CACNG3 | calcium channel, | −2.46 | 0.001 | −3.73 | <0.001 | Plasma | ion channel |
| | voltage- | | | | | Membrane | |
| | dependent, | | | | | | |
| | gamma subunit 3 | | | | | | |
| CALM1 | calmodulin 1 | −1.63 | <0.001 | −1.74 | <0.001 | Plasma | other |
| | (phosphorylase | | | | | Membrane | |
| | kinase, delta) | | | | | | |
| CALM2 | calmodulin 2 | −1.33 | 0.007 | −1.22 | 0.028 | Plasma | other |
| | (phosphorylase | | | | | Membrane | |
| | kinase, delta) | | | | | | |
| CALM3 | calmodulin 3 | −1.52 | 0.005 | −1.34 | 0.01 | Plasma | other |
| | (phosphorylase | | | | | Membrane | |
| | kinase, delta) | | | | | | |
| CALR | calreticulin | 1.19 | 0.016 | 1.18 | 0.023 | Nucleus | transcription |
| | | | | | | | regulator |
| CAMK1 | calcium/calmodulin- | −1.43 | 0.006 | −1.6 | 0.001 | Cytoplasm | kinase |
| | dependent | | | | | | |
| | protein kinase I | | | | | | |
| CAMK4 | calcium/calmodulin- | −1.33 | 0.46 | −1.42 | 0.049 | Nucleus | kinase |
| | dependent | | | | | | |
| | protein kinase IV | | | | | | |
| CAMK1G | calcium/calmodulin- | −2.26 | <0.001 | −2.47 | <0.001 | Plasma | kinase |
| | dependent | | | | | Membrane | |
| | protein kinase IG | | | | | | |
| CAMK2A | calcium/calmodulin- | −4.13 | 0.06 | −2.03 | 0.004 | Cytoplasm | kinase |
| | dependent | | | | | | |
| | protein kinase | | | | | | |
| | (CaM kinase) II | | | | | | |
| | alpha | | | | | | |
| CAMK2B | calcium/calmodulin- | −1.88 | <0.001 | −1.82 | <0.001 | Cytoplasm | kinase |
| | dependent | | | | | | |
| | protein kinase | | | | | | |
| | (CaM kinase) II | | | | | | |
| | beta | | | | | | |
| CAMK2D | calcium/calmodulin- | −1.48 | 0.002 | −1.45 | 0.004 | Cytoplasm | kinase |
| | dependent | | | | | | |
| | protein kinase | | | | | | |
| | (CaM kinase) II | | | | | | |
| | delta | | | | | | |
| CHP | calcium binding | 1.54 | 0.001 | 1.65 | 0.001 | Cytoplasm | transporter |
| | protein P22 | | | | | | |
| CREBBP | CREB binding | 5.84 | 0.004 | 4.14 | 0.009 | Nucleus | transcription |
| | protein | | | | | | regulator |
| | (Rubinstein- | | | | | | |
| | Taybi syndrome) | | | | | | |
| DSCR1 | Down syndrome | −1.82 | <0.001 | −1.93 | <0.001 | Nucleus | transcription |
| | critical region | | | | | | regulator |
| | gene 1 | | | | | | |
| DSCR1L1 | Down syndrome | −4.73 | <0.001 | −4.44 | <0.001 | Unknown | other |
| | critical region | | | | | | |
| | gene 1-like 1 | | | | | | |
| GRIA1 | glutamate receptor, | −2.16 | 0.001 | −2.78 | 0.008 | Plasma | ion channel |
| | ionotropic, AMPA 1 | | | | | Membrane | |
| GRIA2 | glutamate receptor, | 2.71 | 0.002 | 2.74 | <0.001 | Plasma | ion channel |
| | ionotropic, AMPA 2 | | | | | Membrane | |
| GRIN1 | glutamate receptor, | −2.8 | <0.001 | −2.41 | <0.001 | Plasma | ion channel |
| | ionotropic, N- | | | | | Membrane | |
| | methyl D- | | | | | | |
| | aspartate 1 | | | | | | |
| GRIN2B | glutamate receptor, | −1.57 | 0.052 | −1.94 | 0.008 | Plasma | ion channel |
| | ionotropic, N- | | | | | Membrane | |
| | methyl D- | | | | | | |
| | aspartate 2B | | | | | | |
| GRIN3A | glutamate receptor, | −2.94 | 0.063 | −1.84 | 0.013 | Plasma | ion channel |
| | ionotropic, N- | | | | | Membrane | |
| | methyl-D- | | | | | | |
| | aspartate 3A | | | | | | |
| GRINA | glutamate receptor, | −1.2 | 0.009 | −1.21 | 0.007 | Unknown | ion channel |
| | ionotropic, N- | | | | | | |
| | methyl D- | | | | | | |
| | asparate-associated | | | | | | |
| | protein 1 | | | | | | |
| | (glutamate binding) | | | | | | |
| HDAC5 | histone | 1.8 | 0.014 | 1.78 | 0.016 | Nucleus | transcription |
| | deacetylase 5 | | | | | | regulator |
| HDAC6 | histone | −1.13 | 0.015 | −1.18 | 0.005 | Nucleus | transcription |
| | deacetylase 6 | | | | | | regulator |
| HDAC7A | histone | 1.27 | 0.007 | 1.24 | 0.011 | Nucleus | transcription |
| | deacetylase 7A | | | | | | regulator |
| HTR3A | 5- | −1.64 | 0.005 | −1.57 | 0.005 | Plasma | ion channel |
| | hydroxytryptamine | | | | | Membrane | |
| | (serotonin) | | | | | | |
| | receptor 3A | | | | | | |
| ITPR3 | inositol 1,4,5- | −1.93 | 0.002 | −1.85 | 0.019 | Cytoplasm | ion channel |
| | triphosphate | | | | | | |
| | receptor, type 3 | | | | | | |
| MAPK1 | mitogen- | 1.38 | 0.009 | 1.29 | 0.029 | Cytoplasm | kinase |
| | activated protein | | | | | | |
| | kinase 1 | | | | | | |
| MAPK3 | mitogen- | −1.19 | 0.046 | −1.31 | 0.01 | Cytoplasm | kinase |
| | activated protein | | | | | | |
| | kinase 3 | | | | | | |
| MYH1 | myosin, heavy | −2.17 | 0.008 | −2.18 | 0.006 | Cytoplasm | enzyme |
| | polypeptide 1, | | | | | | |
| | skeletal muscle, | | | | | | |
| | adult | | | | | | |
| MYH6 | myosin, heavy | −5.93 | 0.017 | −7.18 | 0.02 | Cytoplasm | other |
| | polypeptide 6, | | | | | | |
| | cardiac muscle, alpha | | | | | | |
| | (cardiomyopathy, | | | | | | |
| | hypertrophic 1) | | | | | | |
| MYH7 | myosin, heavy | −5.88 | <0.001 | −6.11 | <0.001 | Cytoplasm | other |
| | polypeptide 7, | | | | | | |
| | cardiac muscle, | | | | | | |
| | beta | | | | | | |
| MYL6B | myosin, light | −1.68 | <0.001 | −1.69 | 0.001 | Cytoplasm | other |
| | polypeptide 6B, | | | | | | |
| | alkali, smooth | | | | | | |
| | muscle and non- | | | | | | |
| | muscle | | | | | | |
| PPP3CB | protein phosphatase 3 | −1.27 | <0.001 | −1.3 | <0.001 | Unknown | phosphatase |
| | (formerly 2B), | | | | | | |
| | catalytic subunit, | | | | | | |
| | beta isoform | | | | | | |
| | (calcineurin A beta) | | | | | | |
| PPP3CC | protein phosphatase 3 | −1.27 | 0.007 | −1.18 | 0.037 | Unknown | phosphatase |
| | (formerly 2B), | | | | | | |
| | catalytic subunit, | | | | | | |
| | gamma isoform | | | | | | |
| | (calcineurin A gamma) | | | | | | |
| PPP3R1 | protein phosphatase 3 | −1.44 | 0.039 | −1.49 | 0.031 | Cytoplasm | phosphatase |
| | (formerly 2B), regulatory | | | | | | |
| | subunit B, 19 kDa, | | | | | | |
| | alpha isoform | | | | | | |
| | (calcineurin B, type I) | | | | | | |
| PRKAG1 | protein kinase, | −1.24 | 0.005 | −1.21 | 0.012 | Unknown | kinase |
| | AMP-activated, | | | | | | |
| | gamma 1 non- | | | | | | |
| | catalytic subunit | | | | | | |
| PRKAR1A | protein kinase, | −1.1 | 0.001 | 1.08 | 0.015 | Cytoplasm | kinase |
| | cAMP-dependent, | | | | | | |
| | regulatory, type, I | | | | | | |
| | alpha (tissue specific | | | | | | |
| | extinguisher 1) | | | | | | |
| PRKAR1B | protein kinase, | −3.18 | <0.001 | −3.36 | <0.001 | Cytoplasm | kinase |
| | cAMP-dependent, | | | | | | |
| | regulatory, type | | | | | | |
| | I, beta | | | | | | |
| PRKAR2A | protein kinase, | −1.23 | 0.26 | −1.56 | 0.047 | Cytoplasm | kinase |
| | cAMP-dependent, | | | | | | |
| | regulatory, type | | | | | | |
| | II, alpha | | | | | | |
| PRKAR2B | protein kinase, | −1.5 | <0.001 | −1.56 | <0.001 | Cytoplasm | kinase |
| | cAMP-dependent, | | | | | | |
| | regulatory, type | | | | | | |
| | II, beta | | | | | | |
| RAP1B | RAP1B, | −1.33 | 0.002 | −1.29 | <0.001 | Cytoplasm | enzyme |
| | member of RAS | | | | | | |
| | oncogene family | | | | | | |
| TPM1 | tropomyosin 1 | −2.83 | 0.002 | −2.55 | <0.001 | Cytoplasm | other |
| | (alpha) | | | | | | |
| TPM3 | tropomyosin 3 | −1.99 | 0.001 | −2.04 | <0.001 | Cytoplasm | other |
| TRPC1 | transient receptor | −1.08 | 0.33 | −1.21 | 0.033 | Plasma | ion channel |
| | potential cation | | | | | Membrane | |
| | channel, subfamily C, | | | | | | |
| | member 1 | | | | | | |
| TRPC3 | transient receptor | −1.95 | 0.003 | −2.4 | 0.015 | Plasma | ion channel |
| | potential cation | | | | | Membrane | |
| | channel, subfamily C, | | | | | | |
| | member 3 | | | | | | |
| TRPC4 | transient receptor | −5.42 | 0.023 | −4.56 | 0.005 | Plasma | ion channel |
| | potential cation | | | | | Membrane | |
| | channel, subfamily C, | | | | | | |
| | member 4 | | | | | | |
| TRPV6 | transient receptor | 1.73 | <0.001 | 1.65 | <0.001 | Plasma | ion channel |
| | potential cation | | | | | Membrane | |
| | channel, subfamily V, | | | | | | |
| | member 6 | | | | | | |

TABLE 3B
Sterol biosynthesis pathway genes

| Gene | | fold change | p-value | fold change | p-value | | |
|---|---|---|---|---|---|---|---|
| Symbol | Gene Name | 2 vs DMSO | 2 vs DMSO | 1 vs DMSO | 1 vs DMSO | location | family |
| CYP27B1 | cytochrome | −1.38 | 0.008 | −1.27 | 0.055 | Cytoplasm | enzyme |
| | P450, family 27, | | | | | | |
| | subfamily B, | | | | | | |
| | polypeptide 1 | | | | | | |
| DHCR7 | 7- | 1.73 | 0.001 | 1.84 | <0.001 | Cytoplasm | enzyme |
| | dehydrocholesterol | | | | | | |
| | reductase | | | | | | |
| EBP | emopamil | 1.32 | 0.034 | 1.45 | 0.012 | Cytoplasm | enzyme |
| | binding protein | | | | | | |
| | (sterol | | | | | | |
| | isomerase) | | | | | | |
| FDFT1 | farnesyl- | 1.89 | <0.001 | 1.94 | <0.001 | Cytoplasm | enzyme |
| | diphosphate | | | | | | |
| | farnesyl- | | | | | | |
| | transferase 1 | | | | | | |
| FDPS | farnesyl diphosphate | 2.07 | <0.001 | 2.34 | <0.001 | Cytoplasm | enzyme |
| | synthase (farnesyl | | | | | | |
| | pyrophosphate | | | | | | |
| | synthetase, | | | | | | |
| | dimethylallyl- | | | | | | |
| | transtransferase, | | | | | | |
| | geranyltranstransferase) | | | | | | |
| HMGCR | 3-hydroxy-3- | 2.26 | <0.001 | 2.24 | <0.001 | Cytoplasm | enzyme |
| | methylglutaryl- | | | | | | |
| | Coenzyme A | | | | | | |
| | reductase | | | | | | |
| IDI1 | isopentenyl- | 2.85 | <0.001 | 3.24 | <0.001 | Cytoplasm | enzyme |
| | diphosphate | | | | | | |
| | delta isomerase 1 | | | | | | |
| LSS | lanosterol | 13.19 | 0.009 | 13.3 | 0.009 | Cytoplasm | enzyme |
| | synthase (2,3- | | | | | | |
| | oxidosqualene- | | | | | | |
| | lanosterol | | | | | | |
| | cyclase) | | | | | | |
| MVD | mevalonate | 1.81 | 0.005 | 1.94 | 0.004 | Cytoplasm | enzyme |
| | (diphospho) | | | | | | |
| | decarboxylase | | | | | | |
| MVK | mevalonate | 1.6 | 0.001 | 1.6 | <0.001 | Cytoplasm | kinase |
| | kinase | | | | | | |
| | (mevalonic | | | | | | |
| | aciduria) | | | | | | |
| NQO1 | NAD(P)H | 1.64 | 0.001 | 1.8 | <0.001 | Cytoplasm | enzyme |
| | dehydrogenase, | | | | | | |
| | quinone 1 | | | | | | |
| PMVK | phosphomevalonate | 1.25 | 0.024 | 1.33 | 0.009 | Cytoplasm | kinase |
| | kinase | | | | | | |
| SC5DL | sterol-C5- | 1.94 | <0.001 | 2.01 | 0.001 | Cytoplasm | enzyme |
| | desaturase | | | | | | |
| | (ERG3 delta-5- | | | | | | |
| | desaturase | | | | | | |
| | homolog, | | | | | | |
| | fungal)-like | | | | | | |
| SQLE | squalene | 1.9 | <0.001 | 1.92 | <0.001 | Cytoplasm | enzyme |
| | epoxidase | | | | | | |

### Example 10

### Reduction of Subunit of Voltage-Gated L-Type Calcium Channel Stimulates Neurite Outgrowth

RNAi technology was used to reduce the transcription levels of the CACB1 (Ca²⁺ channel β1 subunit) and the FKBP4 (FKBP52) genes, and the biological effect was examined by growth phenotype.

### Methods.

### Neuronal Cultures

Briefly, cortical neuron cultures were prepared from embryonic day 15 (E15) rat embryos (Sprague-Dawley, Charles River Laboratories, Wilmington, Mass.). The embryos were collected, their brains were removed, and the cortices were dissected out in ice-cold phosphate-buffered saline (PBS) without Ca²⁺ and Mg²⁺. Dissected pieces of cortical tissue were pooled together and transferred to an enzymatic dissociation media containing 20 IU/ml papain in Earle's balanced salt solution (Worthington Biochemical, Freehold, N.J.) and incubated for 30 min at 37° C. After enzymatic dissociation, the papain solution was aspirated and the tissue mechanically triturated with a fire-polished Pasteur pipette in complete media [Neurobasal Medium with B-27 supplement (Gibco, Grand Island, N.Y.), 100 IU/ml penicillin, 100 μg/ml streptomycin, 3.3 μg/ml aphidicolin, 0.5 mM glutamate] containing 2,000 IU/ml DNase and 10-mg/ml ovomucoid protease inhibitor.

Transient Transfection of siRNA into Primary Cortical Neurons

For each condition, 5×10⁵ cortical neurons were transfected with 200 ng of siGLO Lamin A/C siRNA (Dharmacon RNA Technologies, Boulder, Colo.), L-type calcium channel β1 subunit siRNA (GGAGAAGUACAAUAAUGACTT (SEQ ID NO:15) (sense) and GUCAUUAUUGUACUUCUCCTT (SEQ ID NO:16) (antisense)) or FKBP4 siRNA (CCUAGCUAUGCUUUUGGCATT (SEQ ID NO:17) (sense) AND UGCCAAAGCAUAGCUAGGTT (SEQ ID NO:18)(antisense) (Ambion, Inc., Austin, Tex.) using program DC-104 on the 96-well shuttle (amaxa biosystems, Gaithersburg, Md.). 25 μl from each transfection reaction were added to a poly-D-lysine-coated 96 well ((4 wells per experiment). Transfected cortical neurons were maintained in culture for 24 h.

### Western Blotting

Cortical neurons treated with scrambled siRNA, lamin A/C, CACNB1, or FKBP52 siRNA were lysed in RIPA buffer containing protease inhibitor cocktail and phosphatase inhibitors and protein concentrations were measured using a Bradford assay (Bio-Rad Laboratories, Hercules, Calif.). 2 μg of protein per condition were loaded into each well and separated via SDS-PAGE. Proteins were transferred onto nitrocellulose and incubated with an antibody against lamin A/C (Upstate), CACNB1 (abcam, Cambridge, Mass.), or FKBP52 (Santa Cruz Biotechnology, Inc.) and actin (Sigma) as a loading control. Bands were developed and quantified using an Odyssey Infrared Imaging System and Odyssey software (Li-Cor Biosciences, Lincoln, Nebr.). Protein expression knock down was calculated as the ratio to actin as a percentage of scrambled siRNA expression.

### Results

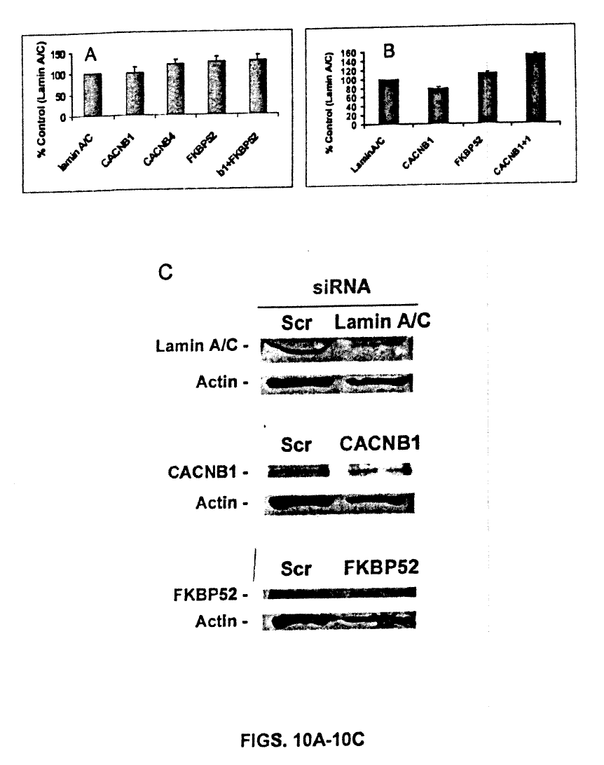

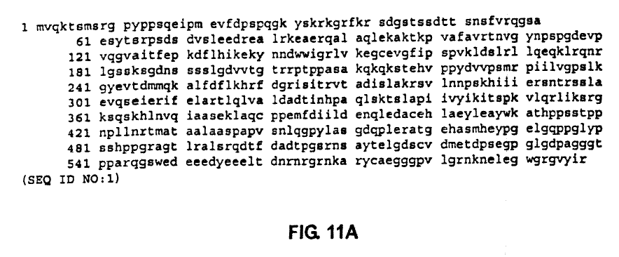

To further demonstrate that inhibition of both FKBP52 and CACNB1 by rapamycin analogue I or II contributes to the neurite outgrowth and neuronal survival, we transfected rat cortical neurons with siRNA against lamin A/C (to serve as a control), FKBP52, CACNB1, or FKBP52+CACNB1 and measured total neurite outgrowth after 24 h. Total neurite outgrowth compared to control was essentially unchanged in CACNB1 siRNA-treated neurons, but significantly increased in FKBP52 siRNA- (125±12% of control) and FKBP52+CACNB1 siRNA-treated (126±14% of control) neurons (FIG. 10A), indicating inhibition of FKBP52 stimulates neurite outgrowth. In parallel, we assessed the effects of siRNA on neuronal survival by an ELISA assay to quantify neurofilament expression. Percent neuronal survival compared to control was decreased in CACNB1 siRNA-treated cells (80±3% of control) and mildly increased in FKBP52 siRNA- (112±2% of control) and significantly in FKBP52+CACNB1 siRNA-treated (152±2% of control) cells (FIG. 10B), indicating that reducing both FKBP52 and CACNB1 promotes neuronal survival. Western blots were performed to verify that siRNA treatment reduced lamin A/C, CACNB1 or FKBP52 protein expression in cortical neurons after 24 h. A representative blot is shown in FIG. 10C. Lamin A/C expression was reduced by 79.21±13.68%, CACNB1 expression was reduced by 70.79±20.79% and FKBP52 expression was reduced by 86.83±7.03% (n=3).

These experiments demonstrate that rapamycin analogue I forms a novel complex with FKBP52 and the voltage gated L-type calcium channel β1 subunit. The complex formation inhibited the activity of the β1 subunit, and stimulated neurite outgrowth. They also demonstrate that two substantially non-immunosuppressive immunophilin ligands, rapamycin analogues I and II, prepared by modification of rapamycin at the mTOR binding region (Abraham et al., Annu. Rev. Immunol. 14, 483-510 (1996)), demonstrated potent neurite outgrowth activity. Affinity purification revealed that both bound to the immunophilin FKBP52 and the β1-subunit of L-type voltage dependent Ca²⁺ channels (CACNB1). Rapamycin analogue II showed 687-fold higher binding selectivity for FKBP52 versus FKBP12 than that of rapamycin. Further more, rat cortical neurons treated with the compounds demonstrated an overall down regulation of Ca²⁺ signaling pathways, and partial inhibition of L-type Ca²⁺ channel was observed in treated F-11 cells. Genetic reduction of FKBP52 and/or CACNB1 in rat cortical neurons promoted neurite outgrowth and neuronal survival. Without being bound to theory, Applicants believe that immunophilin ligands can potentially protect neurons from Ca²⁺ induced cell death by modulating Ca²⁺ signaling, and promote neurite outgrowth by activation of steroid receptors via FKBP52 binding. This novel mechanism of neuroprotective action provides valuable insights for the treatment of many diseases.

The contents of all references, pending patent applications and published patents, cited throughout this application are hereby expressly incorporated by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A purified complex comprising an immunophilin ligand, and one or both of (i) an immunophilin or a functional fragment thereof and/or (ii) a calcium channel subunit or a functional fragment thereof.

2. The purified complex of claim 1, wherein the immunophilin ligand is a rapamycin analogue having a heteroatom substituent at positions 1 and 4 of the rapamycin backbone.

3. The purified complex of claim 1, wherein the immunophilin ligand is a rapamycin analogue having the formula I: 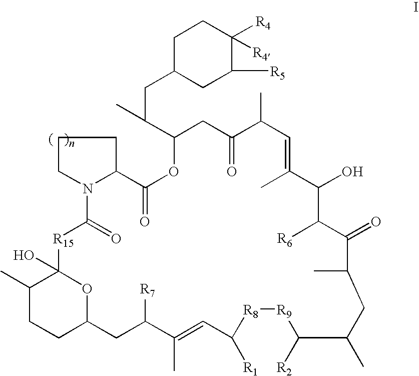
wherein:
R₁ and R₂ are different, independent groups and are selected from the group consisting of OR₃ and N(R_{3′})(R_{3″}); or
R₁ and R₂ are different, are connected through a single bond, and are selected from the group consisting of O and NR₃;
R₃, R_{3′}, and R_{3″} are independently selected from the group consisting of H, C₁ to C₆ alkyl, C₁ to C₆ substituted alkyl, C₃ to C₈ cycloalkyl, substituted C₃ to C₈ cycloalkyl, aryl, substituted aryl, heteroaryl, and substituted heteroaryl;
R₄ and R_{4′} are:
(a) independently selected from the group consisting of H, OH, O(C₁ to C₆ alkyl), O(substituted C₁ to C₆ alkyl), O(acyl), O(aryl), O(substituted aryl), and halogen; or
(b) taken together to form a double bond to O;
R₅, R₆, and R₇ are independently selected from the group consisting of H, OH, and OCH₃;
R₈ and R₉ are connected through a (i) single bond and are CH₂ or (ii) double bond and are CH;
R₁₅ is selected from the group consisting of C═O, CHOH, and CH₂; n is 1 or 2; or a pharmaceutically acceptable salt thereof.

4. The purified complex of claim 3, wherein R₁ of the rapamycin analogue is O, and R₂ is NR₃.

5. The purified complex of claim 3, wherein R₁ of the rapamycin analogue is OR₃ and R₂ is N(R_{3′})(NR_{3″}).

6. The purified complex of claim 3, wherein R₃, R_{3′} or R_{3″} of the rapamycin analogue is an aryl or substituted aryl.

7. The purified complex of claim 6, wherein said aryl or substituted aryl of the rapamycin analogue is of the structure: 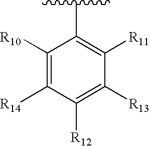
wherein:
R₁₀, R₁₁, R₁₂, R₁₃, and R₁₄ are independently selected from the group consisting of H, C₁ to C₆ alkyl, substituted C₁ to C₆ alkyl, aryl, substituted aryl, heteroaryl, substituted heteroaryl, halogen, acyl, OH, O(alkyl), O(substituted alkyl), O(aryl), O(substituted aryl), O(acyl), NH₂, NH(alkyl), NH(substituted alkyl), NH(aryl), NH(substituted aryl), and NH(acyl).

8. The purified complex of claim 1, wherein the immunophilin ligand is a rapamycin analogue selected from the group consisting of:

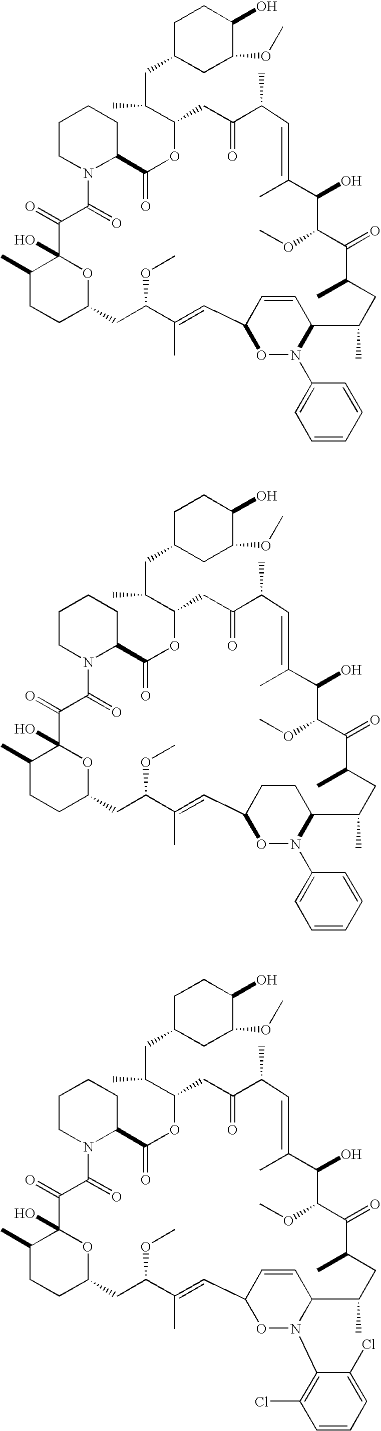

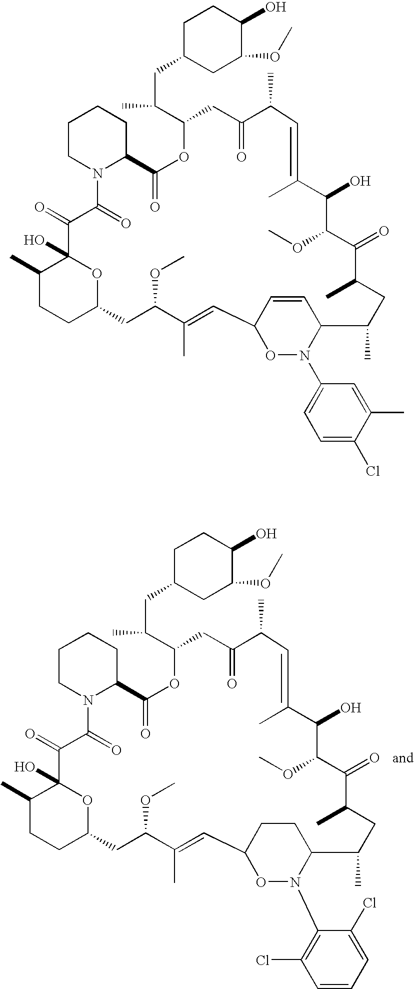

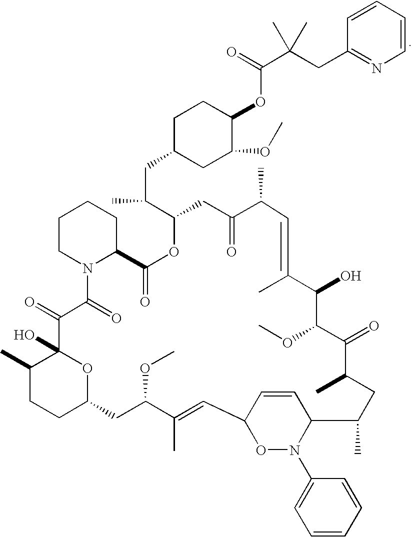

9. The purified complex of claim 1, wherein the immunophilin is FKBP52 or a functional fragment thereof having a sequence at least 95% identical, or identical, to the amino acid sequence shown in FIGS. 12A-12D (SEQ ID NO:11-14).

10. The purified complex of claim 1, wherein the calcium channel subunit is a β1 subunit of the voltage gated L-type calcium channel, or a functional fragment thereof, having a sequence at least 95% identical, or identical, to the amino acid sequence shown in FIGS. 11A-11J (SEQ ID NO:1-10).

11. A recombinant host cell comprising a first recombinant nucleic acid that comprises a nucleotide sequence encoding an FKBP52 having the amino acid sequence shown in FIGS. 12A-12D (SEQ ID NO:11-14, and/or a second recombinant nucleic acid that comprises a nucleotide sequence encoding a β1 subunit of the voltage gated L-type calcium channel having the amino acid sequence shown in FIGS. 11A-11J (SEQ ID NO:1-10).

12. An antibody, or antigen-binding fragment thereof, that binds to the purified complex of claim 1.

13. A method for identifying a test compound that increases the formation of a complex that includes the test compound, and one or both of (i) an immunophilin and/or (ii) a β1 subunit of the voltage gated L-type calcium channel, comprising:
contacting an immunophilin or a functional fragment thereof, and/or a β1 subunit or a functional fragment thereof, with a test compound under conditions that allow formation of the complex;
detecting the presence of the complex in the presence of the test compound relative to a reference;
wherein an increase in the level of the complex in the presence of the test compound, relative to the level of the complex in the reference, indicates that said test compound increases complex formation.

14. The method of claim 13, wherein the sample is a cell lysate, a reconstituted system, comprises cells in culture or in an animal subject.

15. The method of claim 13, wherein the increase in the formation of the complex is determined by detecting one or more of: an increase in the physical formation of the complex, a change in signal transduction, a decrease in calcium channel activity or a change in neuronal activity.

16. The method of claim 15, wherein the change in neuronal activity is detected as an increase in one or more of survival, differentiation or neurite outgrowth.

17. The method of claim 13, wherein the test compound is a polyketide obtained from naturally occurring or modified S. hygroscopicus.

18. A compound identified by the method of claim 13.

19. A method of increasing the formation of a complex that includes an immunophilin ligand, and one or both of (i) an immunophilin or a functional variant thereof and/or (ii) a calcium channel subunit or a functional variant thereof, comprising: contacting an immunophilin or a functional fragment thereof, and/or a β1 subunit of the voltage gated L-type calcium channel or a functional fragment thereof, with an immunophilin ligand, under conditions that increase formation of the complex.

20. The method of claim 19, wherein the contacting step occurs in a cell lysate, in a reconstituted system, or cells in culture or in an animal subject.

21. A method of decreasing voltage-gated calcium channel activity, and/or FKBP52 activity, in a cell, comprising, contacting a cell that expresses one or both of an FKBP52 or a functional fragment thereof, and/or a β1 subunit of the voltage gated L-type calcium channel or a functional fragment thereof, with an immunophilin ligand under conditions that allow binding between the immunophilin ligand, and one or both of the FKBP52 or fragment thereof, and/or the subunit or fragment thereof, to occur, thereby inhibiting the calcium channel activity.

22. The method of claim 21, wherein the contacting step comprises adding the immunophilin ligand to mammalian neuronal or cardiovascular cells in culture.

23. The method of claim 21, wherein the contacting step comprises administration to a subject the immunophilin ligand in an amount sufficient to form a complex between the immunophilin ligand, and one or both of the FKBP52 or fragment thereof, and/or the subunit or fragment thereof.

24. The method of claim 23, wherein the amount of the immunophilin administered to the subject is determined by testing in vitro the amount of immunophilin ligand required to induce complex formation.

25. The method of claim 23 further comprising identifying a subject at risk of having, or having, one or more symptoms associated with a disorder involving L-type calcium channel dysfunction.

26. The method of claim 23, wherein the subject is a mammal suffering from a neurodegenerative or a cardiovascular disorder.

27. The method of claim 23, wherein the immunophilin ligand is administered in combination with an L-type calcium channel antagonist.

28. The method of claim 23, wherein the immunophilin ligand is a rapamycin analogue having a heteroatom substituent at positions 1 and 4 of the rapamycin backbone.

29. The method of claim 28, wherein the rapamycin analogue has the formula I: 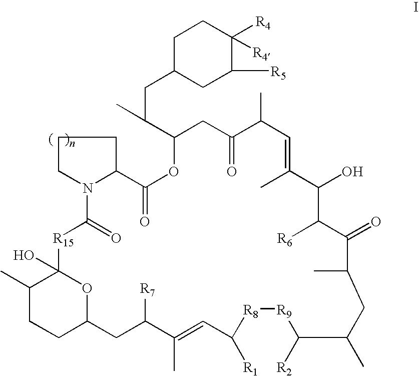
wherein:
R₁ and R₂ are different, independent groups and are selected from the group consisting of OR₃ and N(R_{3′})(R_{3″}); or
R₁ and R₂ are different, are connected through a single bond, and are selected from the group consisting of O and NR₃;
R₃, R_{3′}, and R_{3″} are independently selected from the group consisting of H, C₁ to C₆ alkyl, C₁ to C₆ substituted alkyl, C₃ to C₈ cycloalkyl, substituted C₃ to C₈ cycloalkyl, aryl, substituted aryl, heteroaryl, and substituted heteroaryl;
R₄ and R_{4′} are:
(a) independently selected from the group consisting of H, OH, O(C₁ to C₆ alkyl), O(substituted C₁ to C₆ alkyl), O(acyl), O(aryl), O(substituted aryl), and halogen; or
(b) taken together to form a double bond to O;
R₅, R₆, and R₇ are independently selected from the group consisting of H, OH, and OCH₃;
R₈ and R₉ are connected through a (i) single bond and are CH₂ or (ii) double bond and are CH;
R₁₅ is selected from the group consisting of C═O, CHOH, and CH₂; n is 1 or 2; or a pharmaceutically acceptable salt thereof.

30. The method of claim 29, wherein the rapamycin analogue is selected from the group consisting of:

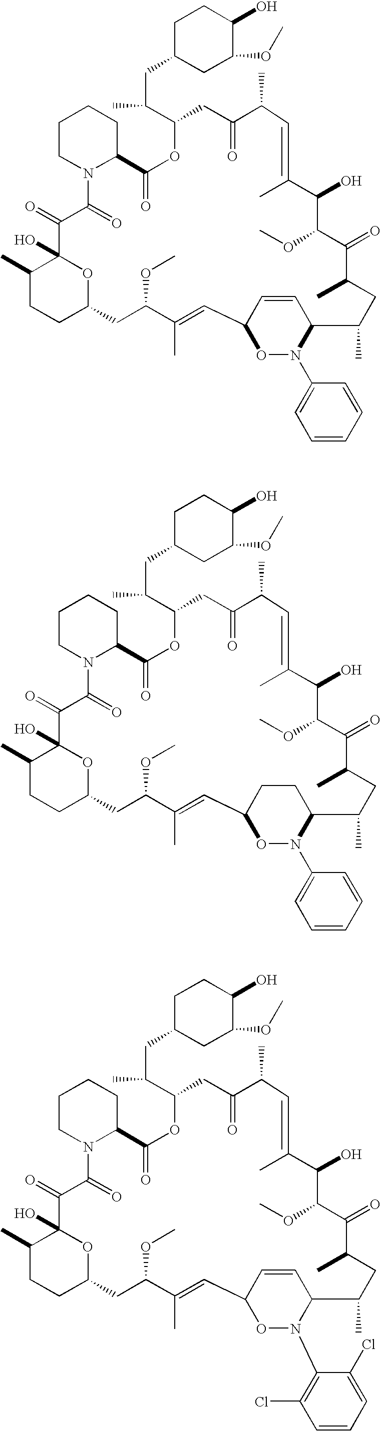

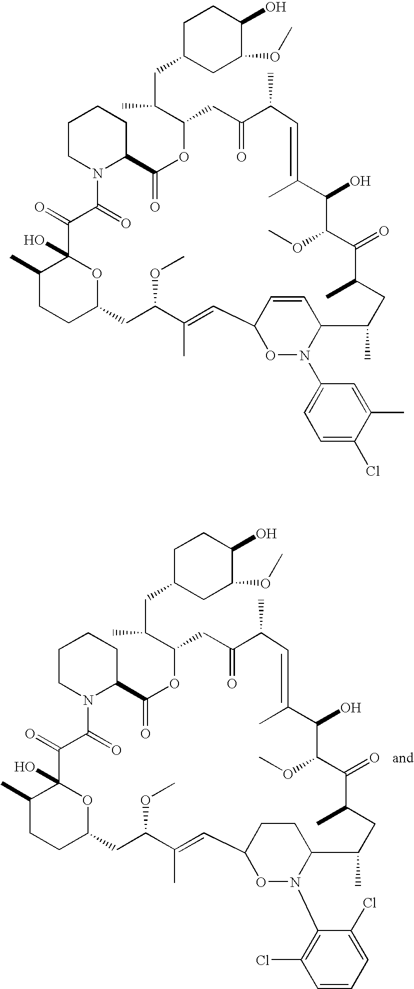

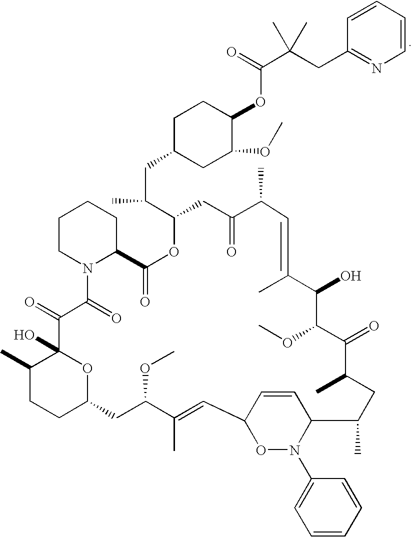

31. The method of claim 21, wherein the FKBP52 or a functional fragment thereof comprises an amino acid sequence at least 95% identical, or identical, to the amino acid sequence shown in FIGS. 12A-12D (SEQ ID NOs:11-12).

32. The method of claim 21, wherein the β1 subunit of the voltage gated L-type calcium channel, or a functional fragment thereof, comprises an amino acid sequence at least 95% identical to the amino acid sequence shown in FIGS. 11A-11J (SEQ ID NOs:1-10).

33. A method of stimulating neurite outgrowth and/or survival of a neuronal cell, comprising, contacting the neuronal cell with an immunophilin ligand, wherein the immunophilin ligand is present at a concentration that elicits one or more of the following: (i) downregulates expression or activity at least one component of the calcium signaling pathways; (ii) decreases FKBP52 activity or expression; (iii) reduces or inhibits the activity or expression of an L-type calcium channel; (iv) activates glucocorticoid receptor signaling; (v) induces formation of a complex that comprises the immunophilin ligand, FKBP52 and/or a β1 subunit; and/or (vi) protects neurons from calcium-induced cell death.

34. The method of claim 33, wherein the contacting step comprises administration to a subject of the immunophilin ligand in an amount sufficient to form the complex that comprises the immunophilin ligand, and one or both of FKBP52 and/or a β1 subunit.

35. The method of claim 34, wherein the amount of the immunophilin administered to the subject is determined by testing in vitro the amount of immunophilin ligand required to induce complex formation.

36. The method of claim 33 further comprising identifying a subject at risk of having, or having, one or more symptoms associated with a disorder involving L-type calcium channel dysfunction.

37. A method of treating a disorder associated with L-type calcium channel dysfunction, comprising administering to a subject an immunophilin ligand in an amount sufficient to form a complex that includes the immunophilin ligand, and one or both of an immunophilin or a functional fragment thereof, and/or a calcium channel subunit or a functional fragment thereof, thereby treating the disorder.

38. The method of claim 37, wherein the amount of the immunophilin administered to the subject is determined by testing in vitro the amount of immunophilin ligand required to induce complex formation.

39. The method of claim 37, further comprising identifying a subject at risk of having, or having, one or more symptoms associated with a disorder involving L-type calcium channel dysfunction.

40. The method of claim 37, wherein the subject is a mammal suffering from a neurodegenerative or a cardiovascular disorder.

41. The method of claim 40, wherein the subject is a mammal suffering from a disorder selected from the group consisting of stroke, Parkinson's disease, epilepsy, angina, cardiac arrhythmia and ischemia.

42. The method of claim 40, wherein the subject is a mammal suffering from a disorder selected from the group consisting of migraine, neuropathic pain, acute pain, mood disorder, schizophrenia, depression, anxiety, cerebellar ataxia, tardive dyskinesia, hypertension and urinary incontinence.

43. The method of claim 37, wherein the immunophilin ligand is administered in combination with an L-type calcium channel antagonist.

44. The method of either claim 33 or 37, wherein the immunophilin ligand is a rapamycin analogue having the formula I: 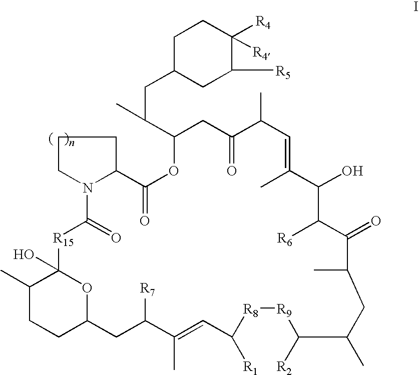
wherein:
R₁ and R₂ are different, independent groups and are selected from the group consisting of OR₃ and N(R_{3′})(R_{3″}); or
R₁ and R₂ are different, are connected through a single bond, and are selected from the group consisting of O and NR₃;
R₃, R_{3′}, and R_{3″} are independently selected from the group consisting of H, C₁ to C₆ alkyl, C₁ to C₆ substituted alkyl, C₃ to C₈ cycloalkyl, substituted C₃ to C₈ cycloalkyl, aryl, substituted aryl, heteroaryl, and substituted heteroaryl;
R₄ and R_{4′} are:
(a) independently selected from the group consisting of H, OH, O(C₁ to C₆ alkyl), O(substituted C₁ to C₆ alkyl), O(acyl), O(aryl), O(substituted aryl), and halogen; or
(b) taken together to form a double bond to O;
R₅, R₆, and R₇ are independently selected from the group consisting of H, OH, and OCH₃;
R₈ and R₉ are connected through a (i) single bond and are CH₂ or (ii) double bond and are CH;
R₁₅ is selected from the group consisting of C═O, CHOH, and CH₂; n is 1 or 2; or a pharmaceutically acceptable salt thereof.

45. The method of claim 44, wherein the rapamycin analogue is selected from the group consisting of:

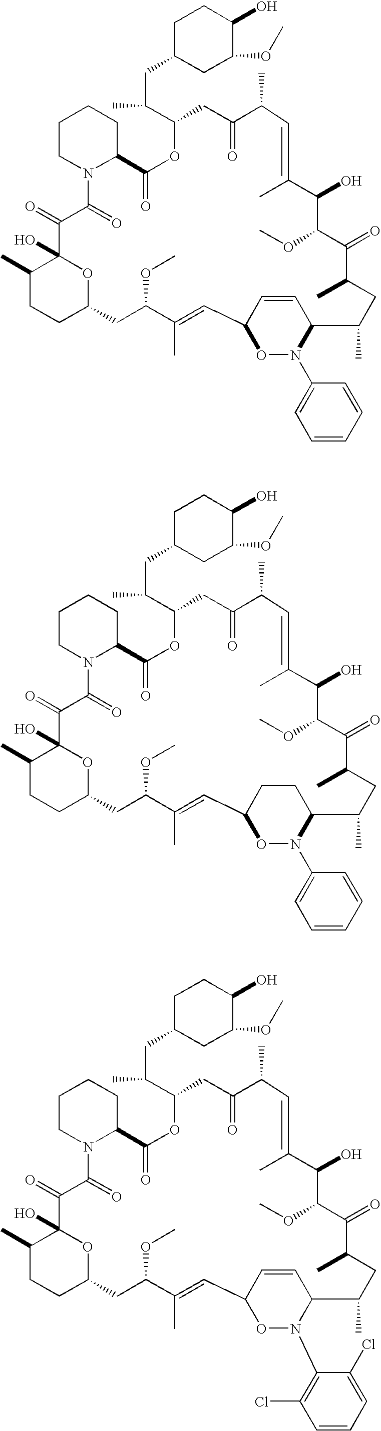

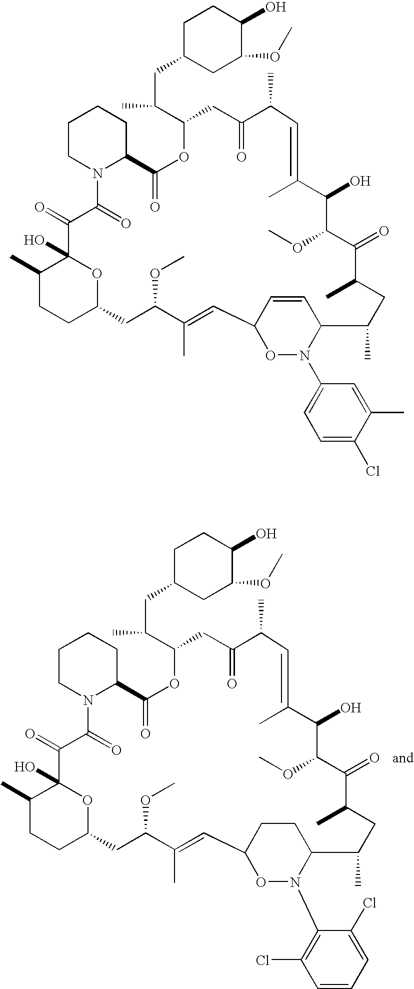

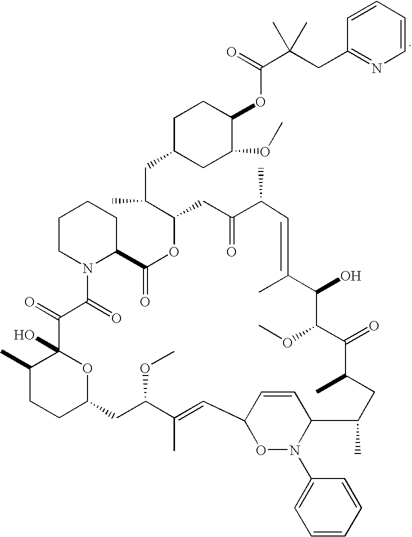

46. The method of claim 44, wherein the immunophilin is FKBP52 or a functional fragment thereof having an amino acid sequence at least 95% identical, or identical, to 167 the amino acid sequence shown in FIGS. 12A-12D (SEQ ID NO:11-14).

47. The method of claim 44, wherein the calcium channel subunit is a β1 subunit of the voltage gated L-type calcium channel, or a functional fragment thereof having an amino acid sequence at least 95% identical, or identical, to the amino acid sequence shown in FIGS. 11A-11J (SEQ ID NO:1-10).

48. A method of stimulating neurite outgrowth of a neuronal cell, comprising contacting the neuronal cell with one or both of an antagonist of a β1 subunit of a voltage gated L-type calcium channel, and/or an antagonist of FKBP52, under condition that reduce the activity or expression of the β1 subunit or FKBP52.

49. The method of claim 48, wherein the neuronal cell is selected from the group consisting of a dopaminergic, a cholinergic, a cortical, and a spinal cord cell.

50. The method of claim 48, wherein the antagonist is an immunophilin ligand that forms a complex with the β1 subunit and/or FKBP52.

51. The method of claim 48, wherein the antagonist is an inhibitor of transcription of the calcium channel β subunit or FKBP52.

52. The method of claim 48, wherein the antagonist is an antibody.

53. Use of an immunophilin ligand in the manufacture of a medicament for the prophylaxis or treatment of a condition associated with L-type calcium channel dysfunction.

54. The use according to claim 53, wherein the immunophilin ligand is a rapamycin analogue having a heteroatom substituent at positions 1 and 4 of the rapamycin backbone.

55. Use of an immunophilin ligand in combination with an L-type calcium channel antagonist for the prophylaxis or treatment of a condition associated with L-type calcium channel dysfunction.

56. Use of a compound identified according to any of claims 13-17 in the manufacture of a medicament for the prophylaxis or treatment of a condition associated with L-type calcium channel dysfunction.

57. An immunophilin ligand for use in the prophylaxis or treatment of a condition associated with L-type calcium channel dysfunction.

58. A composition comprising an immunophilin ligand and an L-type calcium channel antagonist for use in the prophylaxis or treatment of a condition associated with L-type calcium channel dysfunction.

59. A compound identified according to any of claims 13-17 for use in the prophylaxis or treatment of a condition associated with L-type calcium channel dysfunction.


SEQUENCE LISTING (18 sequences)

SEQ ID NO: 1 - protein, 598 aa, Homo sapiens
MVQKTSMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQKLRQNRLGSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQGPYLASGDQPLERATGEHASMHEYPGELGQPPGLYPSSHPPGRAGTLRALSRQDTFDADTPGSRNSAYTELGDSCVDMETDPSEGPGLGDPAGGGTPPARQGSWEDEEEDYEEELTDNRNRGRNKARYCAEGGGPVLGRNKNELEGWGRGVYIR

SEQ ID NO: 2 - DNA, 3687 bp, Homo sapiens
gagggaaggcaggaaggaggcagccgaaggccgagctgggtggctggaccgggtgctggctgcgccgcgctgctttcggctcccacggcctctcccatgcgctgagggagcccggctgggccgggccggcggcgggaggggaggctcctctccatggtccagaagaccagcatgtcccggggcccttacccaccctcccaggagatccccatggaggtcttcgaccccagcccgcagggcaaatacagcaagaggaaagggcgattcaaacggtcagatgggagcacgtcctcggataccacatccaacagctttgtccgccagggctcagcggagtcctacaccagccgtccatcagactctgatgtatctctggaggaggaccgggaagccttaaggaaggaagcagagcgccaggcattagcgcagctcgagaaggccaagaccaagccagtggcatttgctgtgcggacaaatgttggctacaatccgtctccaggggatgaggtgcctgtgcagggagtggccatcaccttcgagcccaaagacttcctgcacatcaaggagaaatacaataatgactggtggatcgggcggctggtgaaggagggctgtgaggttggcttcattcccagccccgtcaaactggacagccttcgcctgctgcaggaacagaagctgcgccagaaccgcctcggctccagcaaatcaggcgataactccagttccagtctgggagatgtggtgactggcacccgccgccccacaccccctgccagtgccaaacagaagcagaagtcgacagagcatgtgcccccctatgacgtggtgccttccatgaggcccatcatcctggtgggaccgtcgctcaagggctacgaggttacagacatgatgcagaaagctttatttgacttcttgaagcatcggtttgatggcaggatctccatcactcgtgtgacggcagatatttccctggctaagcgctcagttctcaacaaccccagcaaacacatcatcattgagcgctccaacacacgctccagcctggctgaggtgcagagtgaaatcgagcgaatcttcgagctggcccggacccttcagttggtcgctctggatgctgacaccatcaatcacccagcccagctgtccaagacctcgctggcccccatcattgtttacatcaagatcacctctcccaaggtacttcaaaggctcatcaagtcccgaggaaagtctcagtccaaacacctcaatgtccaaatagcggcctcggaaaagctggcacagtgcccccctgaaatgtttgacatcatcctggatgagaaccaattggaggatgcctgcgagcatctggcggagtacttggaagcctattggaaggccacacacccgcccagcagcacgccacccaatccgctgctgaaccgcaccatggctaccgcagccctggctgccagccctgcccctgtctccaacctccagggaccctaccttgcttccggggaccagccactggaacgggccaccggggagcacgccagcatgcacgagtacccaggggagctgggccagcccccaggcctttaccccagcagccacccaccaggccgggcaggcacgctacgggcactgtcccgccaagacacttttgatgccgacacccccggcagccgaaactctgcctacacggagctgggagactcatgtgtggacatggagactgacccctcagaggggccagggcttggagaccctgcagggggcggcacgcccccagcccgacagggatcctgggaggacgaggaagaagactatgaggaagagctgaccgacaaccggaaccggggccggaataaggcccgctactgcgctgagggtgggggtccagttttggggcgcaacaagaatgagctggagggctggggacgaggcgtctacattcgctgagaggcaggggccacacggcgggaggaagggctctgagcccaggggaggggagggagcgaggggctcacacctgacatgtattcgcctccagggggcgctgtctccctcctttcagatgcctttgctcaaagcttggggtttctttggtgttaccatcccagctcccgggaggcccttaagccccagctgtcggtttttacctgcctgttgtggatggatgggggatacccacctttctgaagtgtcccctttctcccatcttaaggggctctcctccctcaccctcctagagaaaaggtgcacttccttaactctttctactcggggccctaagtgacggtcctaagtgggatggctctccttctcccaagctgcagtactggggaagggctgggcgcttttcctggaaagggaggccacagattctttcccatgggggctctcttccccagaccccagatccaaggtccctcaccctgcctgccccttcctcccagcttcctggcagcatcgtctggtcggtgaaagccatagcatggacaccccatggggagcttgtcttggggagggttctgggtggaagctggcaggcatacagcaccctctaccctccgtggccatggcaacgtccagggcccagaaccctgaggagtgagcggccgagacgctgctccccaccccccacctccatgcctcagcctttgcctaccccaggaatgagcttggcctccaacatcccttgcctgctgccattagtagagggggcccctctgcatctgagccccccatccctgtgccacctgggtgtggagcccatggaacactctggtccgcctcattttaaaccaaaaaactgctccttcaccctcaccctgaggccccaggggagaggacccgtgggatggtgcccaggggttgccttgggacctcggatctcctctggggggcttggctgctgctgttgctgctctgtatttgcctctcgtgattctgtttgttacccatgttcacttcccccaggagaggccttggtaccccctctcccctggggcatccctttgccttggcatccctgtagcccagcaaccctgcccctccccagcatcccagctgggccagagagagccgagtgtgccaacaaggactggggcctgcccggctgcccgcctcagggatgggcacctcatgcctgtctcgccacctcctgtgccaatgtcccaccctccacctgggggtggggtgcagcttccacttactgattagaagacaccactgccctcccttcccccctccctgtctggtgtcctgtgcccccatctgtctgtctatatttgtctgtactcccctaggagaagtattttgccatatataaaaccactgtcctgtcctttgtggctgcctcccaagcctgcttctttgtcctcgccacatagtcgtcagcgtaggcacctgggagctgctgatatgcacggggagttgaaagggtgggtgcctgaagatgttgtgccctgagtcattgactcaaaagaaaagatgatcctttgattttggccctctgatgtattgtgcccaagccaggagctgcttgggcagtcccagctccacactggccctgagccccttcacttacctgtctctccacaagtagagccaaaggcaatgggaagctcaatgttgctcagtgggtgagatccagacccactggtgcaatgtcttaaatacacatgactgtttttc

SEQ ID NO: 3 - protein, 523 aa, Homo sapiens
MVQKTSMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQKLRQNRLGSSKSGDNSSSSLGDVVTGTRRPTPPASGNEMTNLAFELDPLELEEEEAELGEQSGSAKTSVSSVTTPPPHGKRIPFFKKTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQVQVLTSLRRNLGFWGGLESSQRGSVVPQEQEHAM

SEQ ID NO: 4 - DNA, 1847 bp, Homo sapiens
gagggaaggcaggaaggaggcagccgaaggccgagctgggtggctggaccgggtgctggctgcgccgcgctgctttcggctcccacggcctctcccatgcgctgagggagcccggctgggccgggccggcggcgggaggggaggctcctctccatggtccagaagaccagcatgtcccggggcccttacccaccctcccaggagatccccatggaggtcttcgaccccagcccgcagggcaaatacagcaagaggaaagggcgattcaaacggtcagatgggagcacgtcctcggataccacatccaacagctttgtccgccagggctcagcggagtcctacaccagccgtccatcagactctgatgtatctctggaggaggaccgggaagccttaaggaaggaagcagagcgccaggcattagcgcagctcgagaaggccaagaccaagccagtggcatttgctgtgcggacaaatgttggctacaatccgtctccaggggatgaggtgcctgtgcagggagtggccatcaccttcgagcccaaagacttcctgcacatcaaggagaaatacaataatgactggtggatcgggcggctggtgaaggagggctgtgaggttggcttcattcccagccccgtcaaactggacagccttcgcctgctgcaggaacagaagctgcgccagaaccgcctcggctccagcaaatcaggcgataactccagttccagtctgggagatgtggtgactggcacccgccgccccacaccccctgccagtggtaatgaaatgactaacttagcctttgaactagaccccctagagttagaggaggaagaggctgagcttggtgagcagagtggctctgccaagactagtgttagcagtgtcaccaccccgccaccccatggcaaacgcatccccttctttaagaagacagagcatgtgcccccctatgacgtggtgccttccatgaggcccatcatcctggtgggaccgtcgctcaagggctacgaggttacagacatgatgcagaaagctttatttgacttcttgaagcatcggtttgatggcaggatctccatcactcgtgtgacggcagatatttccctggctaagcgctcagttctcaacaaccccagcaaacacatcatcattgagcgctccaacacacgctccagcctggctgaggtgcagagtgaaatcgagcgaatcttcgagctggcccggacccttcagttggtcgctctggatgctgacaccatcaatcacccagcccagctgtccaagacctcgctggcccccatcattgtttacatcaagatcacctctcccaaggtacttcaaaggctcatcaagtcccgaggaaagtctcagtccaaacacctcaatgtccaaatagcggcctcggaaaagctggcacagtgcccccctgaaatgtttgacatcatcctggatgagaaccaattggaggatgcctgcgagcatctggcggagtacttggaagcctattggaaggccacacacccgcccagcagcacgccacccaatccgctgctgaaccgcaccatggctaccgcagccctggctgccagccctgcccctgtctccaacctccaggtacaggtgctcacctcgctcaggagaaacctcggcttctggggcgggctggagtcctcacagcggggcagtgtggtgccccaggagcaggaacatgccatgtagtgggcgccctgcccgtcttccctcctgctctggggtcggaactggagtgcagggaacatggaggaggaagggaagagctttattttgtaaaaaaataagatgagcggcaaaaaaaaaaaaaa

SEQ ID NO: 5 - protein, 478 aa, Homo sapiens
MVQKTSMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQKLRQNRLGSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQVQVLTSLRRNLGFWGGLESSQRGSVVPQEQEHAM

SEQ ID NO: 6 - DNA, 1700 bp, Homo sapiens
gagggaaggcaggaaggaggcagccgaaggccgagctgggtggctggaccgggtgctggctgcgccgcgctgctttcggctcccacggcctctcccatgcgctgagggagcccggctgggccgggccggcggcgggaggggaggctcctctccatggtccagaagaccagcatgtcccggggcccttacccaccctcccaggagatccccatggaggtcttcgaccccagcccgcagggcaaatacagcaagaggaaagggcgattcaaacggtcagatgggagcacgtcctcggataccacatccaacagctttgtccgccagggctcagcggagtcctacaccagccgtccatcagactctgatgtatctctggaggaggaccgggaagccttaaggaaggaagcagagcgccaggcattagcgcagctcgagaaggccaagaccaagccagtggcatttgctgtgcggacaaatgttggctacaatccgtctccaggggatgaggtgcctgtgcagggagtggccatcaccttcgagcccaaagacttcctgcacatcaaggagaaatacaataatgactggtggatcgggcggctggtgaaggagggctgtgaggttggcttcattcccagccccgtcaaactggacagccttcgcctgctgcaggaacagaagctgcgccagaaccgcctcggctccagcaaatcaggcgataactccagttccagtctgggagatgtggtgactggcacccgccgccccacaccccctgccagtgccaaacagaagcagaagtcgacagagcatgtgcccccctatgacgtggtgccttccatgaggcccatcatcctggtgggaccgtcgctcaagggctacgaggttacagacatgatgcagaaagctttatttgacttcttgaagcatcggtttgatggcaggatctccatcactcgtgtgacggcagatatttccctggctaagcgctcagttctcaacaaccccagcaaacacatcatcattgagcgctccaacacacgctccagcctggctgaggtgcagagtgaaatcgagcgaatcttcgagctggcccggacccttcagttggtcgctctggatgctgacaccatcaatcacccagcccagctgtccaagacctcgctggcccccatcattgtttacatcaagatcacctctcccaaggtacttcaaaggctcatcaagtcccgaggaaagtctcagtccaaacacctcaatgtccaaatagcggcctcggaaaagctggcacagtgcccccctgaaatgtttgacatcatcctggatgagaaccaattggaggatgcctgcgagcatctggcggagtacttggaagcctattggaaggccacacacccgcccagcagcacgccacccaatccgctgctgaaccgcaccatggctaccgcagccctggctgccagccctgcccctgtctccaacctccaggtacaggtgctcacctcgctcaggagaaacctcggcttctggggcgggctggagtcctcacagcggggcagtgtggtgccccaggagcaggaacatgccatgtagtgggcgccctgcccgtcttccctcctgctctggggtcggaactggagtgcagggaacatggaggaggaagggaagagctttattttgtaaaaaaataagatgagcggcaa

SEQ ID NO: 7 - protein, 524 aa, Mus musculus
MVQKSGMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQTLRQNRLSSSKSGDNSSSSLGDVVTGTRRPTPPASGNEMTNFAFELDPLELEEEEAELGEHGGSAKTSVSSVTTPPPHGKRIPFFKKTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQVQVLTSLRRNLSFWGGLEASPRGGDAVAQPQEHAM

SEQ ID NO: 8 - DNA, 1892 bp, Mus musculus
ttccggcggcggcggcggcgacggcggcagcggccgcagagagcacagcgcgagccgggagggcaagcaaggcggcgagcgtgcagccggaggtccagctgggagactgcacccggtgctggctgcgcgacgccgcgctgctctgggctcggacggcctctcccatgcgctgagagcgcccggctgggctgggagggcggccggaccggaggatcctctccatggtccagaagagcggcatgtcccggggcccttacccaccttcccaagagatccctatggaggtcttcgaccccagcccacagggcaagtacagcaagaggaaagggcggttcaaaaggtcagacgggagtacgtcctcggatacaacatccaacagcttcgtccgccagggctcagcagagtcctacacgagccgaccatcagactctgatgtgtctctggaggaggaccgggaagccttaaggaaggaggcagagcgccaggccttagcccagctcgagaaagccaagaccaaaccagtggcttttgctgttcggacaaatgttggctacaatccgtctccaggggatgaggtgcctgtacagggagtggccatcacctttgagcccaaggacttcctacacatcaaggagaagtacaataatgactggtggattgggcggctggtgaaggaaggctgcgaggttggcttcatccccagcccggtcaaactggacagccttcgtctgctgcaggaacagaccctgcgccagaaccgcctcagctccagcaagtcaggtgacaactccagttccagtctgggagatgtggtgactggcacccgccgccccacaccccctgccagtggtaatgaaatgactaactttgcctttgagctagaccccctagagttagaggaggaggaggcagagctaggggagcacggcggctcagccaagactagcgtgagcagtgtcaccacgccgccaccccacggcaagcgcatccccttctttaagaagacagagcacgtgcccccctatgacgtggtgccttccatgaggcccatcatcctggtgggaccgtcgctcaagggctatgaggtgacagacatgatgcagaaagcgttgtttgacttcctcaagcatcggtttgatggcaggatttccatcacccgggtaacagctgacatttccctggccaaacgctccgtcctcaacaaccccagcaaacacatcatcattgagcgctccaacacgcgttccagcctggctgaggtacagagtgaaattgagaggatcttcgagctggcccggaccttgcagctggtcgccttggacgctgacaccatcaaccacccagcccagctctctaaaacgtcgctggcccccatcattgtttacatcaagatcacatctcccaaggtactgcagaggctcatcaaatcccgagggaagtctcaatccaaacacctcaatgtccaaatagcagcctcggagaagctggcacagtgtccccccgaaatgtttgacataatcctggacgagaaccaattggaagatgcctgcgagcacctggctgagtacttggaagcctactggaaggccacacatccgcctagcagcacgccacccaatccgctgctgaaccgcaccatggctaccgcagctctggctgccagccctgcccccgtctccaacctccaggtacaggtgctcacctcgctcaggagaaatctcagcttctggggcgggctggaggcctcaccgcggggaggcgacgcggtggcccagcctcaggagcacgccatgtagccgatgtccctctggtctttcctcccaccctggagtgcagggaacatgaggaaggaagggaagagctttattttgtaaaaaacgtggtgagcggca

SEQ ID NO: 9 - protein, 597 aa, Mus musculus
MVQKSGMSRGPYPPSQEIPMEVFDPSPQGKYSKRKGRFKRSDGSTSSDTTSNSFVRQGSAESYTSRPSDSDVSLEEDREALRKEAERQALAQLEKAKTKPVAFAVRTNVGYNPSPGDEVPVQGVAITFEPKDFLHIKEKYNNDWWIGRLVKEGCEVGFIPSPVKLDSLRLLQEQTLRQNRLSSSKSGDNSSSSLGDVVTGTRRPTPPASAKQKQKSTEHVPPYDVVPSMRPIILVGPSLKGYEVTDMMQKALFDFLKHRFDGRISITRVTADISLAKRSVLNNPSKHIIIERSNTRSSLAEVQSEIERIFELARTLQLVALDADTINHPAQLSKTSLAPIIVYIKITSPKVLQRLIKSRGKSQSKHLNVQIAASEKLAQCPPEMFDIILDENQLEDACEHLAEYLEAYWKATHPPSSTPPNPLLNRTMATAALAASPAPVSNLQGPYLASGDQPLDRATGEHASVHEYPGELGQPPGLYPSNHPLGRAGTLRALSRQDTFDADTPGSRNSAYTEPGDSCVDMETDPSEGPGPGDPAGGGTPPARQGSWEDEEDYEEEMTDNRNRGRNKARYCAEGGGPVLGRNKNELEGWGQGVYTR

SEQ ID NO: 10 - DNA, 1794 bp, Mus musculus
atggtccagaagagcggcatgtcccggggcccttacccaccttcccaagagatccctatggaggtcttcgaccccagcccacagggcaagtacagcaagaggaaagggcggttcaaaaggtcagacgggagtacgtcctcggatacaacatccaacagcttcgtccgccagggctcagcagagtcctacacgagccgaccatcagactctgatgtgtctctggaggaggaccgcgaagccttaaggaaggaggcagagcgccaggccttagcccagctcgagaaagccaagaccaaaccagtggcttttgctgttcggacaaatgttggctacaatccgtctccaggggatgaggtgcctgtacagggagtggccatcacctttgagcccaaggacttcctacacatcaaggagaagtacaataatgactggtggattgggcggctggtgaaggaaggctgcgaggttggcttcatccccagcccggtcaaactggacagccttcgtctgctgcaggaacagaccctgcgccagaaccgcctcagctccagcaagtcaggtgacaactccagttccagtctgggagatgtggtgactggcacccgccgccccacaccccctgccagtgccaaacagaagcagaaatcgacagagcacgtgcccccctatgacgtggtgccttccatgaggcccatcatcctggtgggaccgtcgctcaagggctatgaggtgacagacatgatgcagaaagcgttgtttgacttcctcaagcatcggtttgatggcaggatttccatcacccgggtaacagctgacatttccctggccaaacgctccgtcctcaacaaccccagcaaacacatcatcattgagcgctccaacacgcgttccagcctggctgaggtacagagtgaaattgagaggatcttcgagctggcccggaccttgcagctggtcgccttggacgctgacaccatcaaccacccagcccagctctctaaaacgtcgctggcccccatcattgtttacatcaagatcacatctcccaaggtactgcagaggctcatcaaatcccgagggaagtctcaatccaaacacctcaatgtccaaatagcagcctcggagaagctggcacagtgtccccccgaaatgtttgacataatcctggacgagaaccaattggaagatgcctgcgagcacctggctgagtacttggaagcctactggaaggccacacatccgcctagcagcacgccacccaatccgctgctgaaccgcaccatggctaccgcagctctggctgccagccctgcccccgtctccaacctccagggaccctaccttgcttccggggaccagccgctggaccgggccactggggaacatgccagtgtgcacgagtaccccggggaattgggccagcccccaggcctttaccccagcaaccacccacttggccgggcaggcaccctgcgggcgctatcccgccaagacacctttgatgctgacacccccggcagccgaaattctgcctacacggagccgggagactcgtgtgtggacatggagacagacccctcagagggcccagggcctggagaccctgcagggggaggcacaccaccagcccggcagggctcctgggaagacgaggaagactatgaggaggagatgaccgacaacaggaaccggggccggaataaggcccgctactgtgcggagggtggtgggccggttctggggcgcaataagaatgagctggagggctggggacaaggcgtctacactcgctga

SEQ ID NO: 11 - protein, 457 aa, Homo sapiens
MTTDEGAKNNEESPTATVAEQGEDITSKKDRGVLKIVKRVGNGEETPMIGDKVYVHYKGKLSNGKKFDSSHDRNEPFVFSLGKGQVIKAWDIGVATMKKGEICHLLCKPEYAYGSAGSLPKIPSNATLFFEIELLDFKGEDLFEDGGIIRRTKRKGEGYSNPNEGATVEIHLEGRCGGRMFDCRDVAFTVGEGEDHDIPIGIDKALEKMQREEQCILYLGPRYGFGEAGKPKFGIEPNAELIYEVTLKSFEKAKESWEMDTKEKLEQAAIVKEKGTVYFKGGKYMQAVIQYGKIVSWLEMEYGLSEKESKASESFLLAAFLNLAMCYLKLREYTKAVECCDKALGLDSANEKGLYRRGEAQLLMNEFESAKGDFEKVLEVNPQNKAARLQISMCQKKAKEHNERDRRIYANMFKKFAEQDAKEEANKAMGKKTSEGVTNEKGTDSQAMEEEKPEGHV

SEQ ID NO: 12 - DNA, 3781 bp, Homo sapiens
gggccggctcgcgggcgctgccagtctcgggcggcggtgtccggcgcgcgggcggcctgctgggcgggctgaagggttagcggagcacgggcaaggcggagagtgacggagtcggcgagcccccgcggcgacaggttctctacttaaaagacaatgactactgatgaaggtgccaagaacaatgaagaaagccccacagccactgttgctgagcagggagaggatattacctccaaaaaagacaggggagtattaaagattgtcaaaagagtggggaatggtgaggaaacgccgatgattggagacaaagtttatgtccattacaaaggaaaattgtcaaatggaaagaagtttgattccagtcatgatagaaatgaaccatttgtctttagtcttggcaaaggccaagtcatcaaggcatgggacattggggtggctaccatgaagaaaggagagatatgccatttactgtgcaaaccagaatatgcatatggctcggctggcagtctccctaaaattccctcgaatgcaactctcttttttgagattgagctccttgatttcaaaggagaggatttatttgaagatggaggcattatccggagaaccaaacggaaaggagagggatattcaaatccaaacgaaggagcaacagtagaaatccacctggaaggccgctgtggtggaaggatgtttgactgcagagatgtggcattcactgtgggcgaaggagaagaccacgacattccaattggaattgacaaagctctggagaaaatgcagcgggaagaacaatgtattttatatcttggaccaagatatggttttggagaggcagggaagcctaaatttggcattgaacctaatgctgagcttatatatgaagttacacttaagagcttcgaaaaggccaaagaatcctgggagatggataccaaagaaaaattggagcaggctgccattgtcaaagagaagggaaccgtatacttcaagggaggcaaatacatgcaggcggtgattcagtatgggaagatagtgtcctggttagagatggaatatggtttatcagaaaaggaatcgaaagcttctgaatcatttctccttgctgcctttctgaacctggccatgtgctacctgaagcttagagaatacaccaaagctgttgaatgctgtgacaaggcccttggactggacagtgccaatgagaaaggcttgtataggaggggtgaagcccagctgctcatgaacgagtttgagtcagccaagggtgactttgagaaagtgctggaagtaaacccccagaataaggctgcaagactgcagatctccatgtgccagaaaaaggccaaggagcacaacgagcgggaccgcaggatatacgccaacatgttcaagaagtttgcagagcaggatgccaaggaagaggccaataaagcaatgggcaagaagacttcagaaggggtcactaatgaaaaaggaacagacagtcaagcaatggaagaagagaaacctgagggccacgtatgacgccacgccaaggagggaagagtcccagtgaactcggcccctcctcaatgggctttcccccaactcaggacagaacagtgtttaatgtaaagtttgttatagtctatgtgattctggaagcaaatggcaaaaccagtagcttcccaaaaacagcccccctgctgctgcccggagggttcactgaggggtggcacgggaccactccaggtggaacaaacagaaatgactgtggtgtggagggagtgagccagcagcttaagtccagctcatttcagtttctatcaaccttcaagtatccaattcagggtccctggagatcatcctaacaatgtggggctgttaggttttacctttgaactttcatagcactgcagaaaccttttaaaaaaaaatgcttcatgaatttctcctttcctacagttgggtagggtaggggaaggaggataagcttttgttttttaaatgactgaagtgctataaatgtagtctgttgcatttttaaccaacagaacccacagtagaggggtctcatgtctccccagttccacagcagtgtcacagacgtgaaagccagaacctcagaggccacttgcttgctgacttagcctcctcccaaagtccccctcctcagccagcctccttgtgagagtggctttctaccacacacagcctgtccctgggggagtaattctgtcattcctaaaacacccttcagcaatgataatgagcagatgagagtttctggattagcttttcctattttcgatgaagttctgagatactgaaatgtgaaaagagcaatcagaattgtgctttttctcccctcctctattccttttagggaataatattcaatacacagtacttcctcccagcattgctactgctcagcttcttctttcattctaatccttgctattaagaatttaagacttgtgcttacaatatttttgacctggagtggatctatttacatagtcatttaggatccatgcagctttttttgtctttttaagattattggctcataagcatatgtatactggtttatggaactttatttacactcctctatcatgcaaaaaaattttgactttttagtactaagcttaatttttaaaaacaaaatctgtagtgttgacaaataaatagttgctcttctacactaggggtttcacctgcaggtttgacacgcagttgctcgcttttcctgccctgtcaagcttctctgttctggcgtgagttgtgaaagagttgaagacagcttcccatgccggtacacagccagtagcctaaatctccagtacttgagctgaccattgaactagggcaagtcttaaatgtgtacatgtagttgaatttcagtccttacgggtaaacagattgagcatggctctctattccctcagcctaagaaacactcatgggaatgcatttggcaacccaaggaaccatttgcttaaacctggaacatctcacctttttaaatcctaaaaaacactggcagttatattttaaattagtttttatttttatgatggttttatcaaaagacttttattattagattgggacccccttcaaacctaaaaatcaagttatttccttttataatacttttcttccccatggaacaaatgggatcaatttgtgagttttttcctttaatgataactaaaatccctctaatttctcatttatgcttttgtcttttttatgaaatatttcttttaaaagccccagtctcacctacgaaatatgaagagcaaaagctgattttgcttacttgctaaactgttgggaaagctctgtagagcatggttccagtgaggccaagattgaaatttgatactaaaaaggccacctagctttttgcagataacaaacaagaaagctattccaagactcagatgatgccagctgtctcccacgtgtgtattatggttcaccagggggaactggcaaaagtgtgtgtggggaggggaagggtgtgtgagtggttctgagcaaataactacagggtgcccattaccactcaagaagacacttcacgtattcttgtatcaaattcaataatcttaaacaatttgtgtagaagtccacagacatctttcaaccaccttttaggctgcatatggattgccaagtcagcatatgaggaattaaagacattgtttttaaaaaaaaaaaatcatttagatgcacttttttgtgtgttctttaaataaatccaaaaaaaatgtgaaaaaaaaaa

SEQ ID NO: 13 - protein, 456 aa, Mus musculus
MTTDEGTSNNGENPAATMTEQGEDITTKKDRGVLKIVKRVGTSDEAPMFGDKVYVHYKGMLSDGKKFDSSHDRKKPFAFSLGQGQVIKAWDIGVSTMKKGEICHLLCKPEYAYGSAGHLQKIPSNATLFFEIELLDFKGEDLFEDSGVIRRIKRKGEGYSNPNEGATVKVHLEGCCGGRTFDCRDVVFVVGEGEDHDIPIGIDKALVKMQREEQCILYLGPRYGFGEAGKPKFGIDPNAELMYEVTLKSFEKAKESWEMDTKEKLTQAAIVKEKGTVYFKGGKYTQAVIQYRKIVSWLEMEYGLSEKESKASESFLLAAFLNLAMCYLKLREYNKAVECCDKALGLDSANEKGLYRRGEAQLLMNDFESAKGDFEKVLAVNPQNRAARLQISMCQRKAKEHNERDRRVYANMFKKFAERDAKEEASKAGSKKAVEGAAGKQHESQAMEEGKAKGHV

SEQ ID NO: 14 - protein, 456 aa, Mus musculus; identical to SEQ ID NO: 13

SEQ ID NO: 15 - DNA, 21 bp, Artificial Sequence
  Primer
ggagaaguacaauaaugactt

SEQ ID NO: 16 - DNA, 21 bp, Artificial Sequence
  Primer
gucauuauuguacuucucctt

SEQ ID NO: 17 - DNA, 21 bp, Artificial Sequence
  Primer
ccuagcuaugcuuuuggcatt

SEQ ID NO: 18 - DNA, 20 bp, Artificial Sequence
  Primer
ugccaaagcauagcuaggtt